(19) United States
(12) Patent Application Publication
(10) Pub. No.: US 2024/0238338 A1
(43) Pub. Date: Jul. 18, 2024

(54) THERAPEUTIC APPLICATION OF SKULL BONE MARROW AND BRAIN BORDER-DERIVED CELLS

(71) Applicant: Washington University, St. Louis, MO (US)

(72) Inventor: Jonathan Kipnis, St. Louis, MO (US)

(73) Assignee: Washington University, St. Louis, MO (US)

(21) Appl. No.: 18/560,263

(22) PCT Filed: May 10, 2022
(86) PCT No.: PCT/US22/28405

§ 371 Date: Nov. 10, 2023

Related U.S. Application Data
(60) Provisional application No. 63/186,309, filed on May 10, 2021.

(51) Int. Cl. A61K 35/15; A61P 25/00

(52) U.S. Cl. CPC A61K 35/15; A61P 25/00

(57) ABSTRACT

Compositions and methods for the treatment, prevention, or reversal of a central nervous system (CNS) injury or disorder in a patient in need that include administering a therapeutically effective amount of meningeal immune cells are disclosed. Specifically, the meningeal immune cells comprise Ly6C+ monocytes. Further disclosed are wherein the meningeal immune cells are modified to include a drug or gene therapeutic compound, and wherein the modified meningeal immune cells are configured to deliver the drug or gene therapeutic compound to the CNS of the subject via the parenchyma or meningeal tissue of the subject.

### CROSS-REFERENCE TO RELATED APPLICATIONS

This application claims priority from U.S. Provisional Application Ser. No. 63/186,309 filed on May 10, 2021, which is incorporated herein by reference in its entirety.

### STATEMENT REGARDING FEDERALLY SPONSORED RESEARCH OR DEVELOPMENT

This invention was made with government support under NS096967, AT010416, AG034113, and AG057496 awarded by the National Institutes of Health. The government has certain rights in the invention.

### MATERIAL INCORPORATED-BY-REFERENCE

Not applicable.

### FIELD OF THE DISCLOSURE

The present disclosure generally relates to compositions and methods for the treatment, prevention, or reversal of a CNS injury or disorder.

### BACKGROUND OF THE DISCLOSURE

Over the last two decades, our understanding of myeloid cell biology has been transformed. Macrophages, an essential component of the myeloid niche, represent a dispersed homeostatic organ essential to the daily demands and maintenance of the tissues in which they reside. Research on microglia, the macrophages of the CNS, led to the discovery that these tissue-resident macrophages are derived from the yolk sac during embryonic development. Unlike several other tissue macrophages that can be replaced by blood-borne monocytes, according to specific anatomical and developmental constraints, microglia are self-maintained and are not replenished by blood-derived monocytes other than following injury or neuroinflammation. In such cases, myeloid cells from the periphery may also invade the CNS tissue, although their origin, as well as their stability within the CNS, are poorly understood. The perivascular spaces and the meningeal membranes that cover the borders of the CNS are also populated by a variety of myeloid cells. Based on novel fate-mapping strategies and multidimensional tools, recent works have begun to reveal the richness of this myeloid landscape, both at steady-state and in different diseases. Less well studied, however, is the source and role of these border-resident monocytes, and monocyte-derived macrophages.

Meninges—a membranous structure enveloping the central nervous system (CNS), hosts a rich repertoire of immune cells mediating CNS immune surveillance. Myeloid cells, including monocytes, neutrophils, and macrophages display extraordinary heterogeneity and diverse functions depending on their ontogeny and local niche. Macrophages represent an essential component of the myeloid niche and take on distinct transcriptional signatures to meet the demands of the tissues in which they reside. Research on microglia, the macrophages of the central nervous system (CNS), led to the discovery that these tissue-resident macrophages are derived from the yolk sac during embryonic development. Unlike several other tissue macrophages that can be replaced by blood-borne monocytes, according to specific anatomical and developmental constraints, microglia are self-maintained and are not replenished by blood-derived monocytes other than under defined experimental conditions. In such cases, myeloid cells from the periphery may also invade the CNS tissue, although their origin, as well as their stability within the CNS, are poorly understood. The perivascular spaces and the meningeal membranes that cover the borders of the CNS are also populated by a variety of myeloid cells. Based on elegant fate-mapping strategies and multidimensional tools, recent work has begun to reveal the richness of this myeloid landscape, both at steady-state and in different diseases. Less well studied, however, is the source and role of these brain border-resident monocytes, monocyte-derived macrophages, and neutrophils during homeostasis and CNS dysfunction.

Recent works described the existence of direct ossified vascular channels connecting skull bone marrow to meninges, capable of dispersing neutrophils during inflammation

### DESCRIPTION OF THE DRAWINGS

Those of skill in the art will understand that the drawings, described below, are for illustrative purposes only. The drawings are not intended to limit the scope of the present teachings in any way.

FIGS. 1A-1K overall show CNS borders host a substantial pool of monocytes and neutrophils that is not blood derived. FIG. 1A is the experimental design for parabiosis experiments. WT mice were parabiotically joined with UBC-GFP mice and analyzed 60 days later. FIG. 1B is a representative gating strategy for flow cytometry of immune cells from the cranial dura. FIG. 1C is a representative histogram of GFP− and GFP+Ly6C+ monocytes and neutrophils across tissues. WT and UBC-GFP control mice without IV anti-CD45 antibody injection were used to assign gates. FIG. 1D is a quantification of flow cytometry analysis for GFP+Ly6C+ monocytes across tissues. NS=not significant, ***P<0.001 versus blood (One-way ANOVA with Dunnett's post hoc test), n=8 mice per tissue, representative of 3 independent experiments. Data are means ±SEM. FIG. 1E is a quantification of flow cytometry analysis for GFP+ neutrophils across tissues. NS=not significant, ***P<0.001 versus blood (One-way ANOVA with Dunnett's post hoc test), n=8 mice per tissue, representative of 3 independent experiments. Data are means ±SEM. FIG. 1F is a quantification of flow cytometry analysis for GFP+CD4 T cells across tissues. NS=not significant, ***P<0.001 versus blood (One-way ANOVA with Dunnett's post hoc test), n=8 mice per tissue, representative of 3 independent experiments. Data are means ±SEM. FIG. 1G is representative images from 3 mice of cranial and spinal dura whole mounts from WT parabionts 60 days after parabiotic pairing. Scale bars, (left) 2000 μm; (right) 50 μm. FIG. 1H is the experimental design for myeloid fate mapping strategy using LysM-CreERT2::ZsGreen reporter mice to assess the half-life of meningeal Ly6C+ monocytes and GFP+ neutrophils. Mice underwent 3 daily tamoxifen injections and were then analyzed at 4 different time points. Cre− animals were used as controls to assign gates. FIG. 1 Iis flow cytometry analysis of Ly6C+ monocytes (top) and GFP+ neutrophils (bottom) at 0 and 30 days. Black outlined histograms represent Cre− controls and green histograms represent LysM-CreERT2::ZsGreen reporter mice. FIG. 1J is a graph of the percent change in ZsGreen+Ly6C+ monocytes over time. N=3-4 mice per group, representative of 2 independent experiments. Data are means ±SEM. FIG. 1K is a graph of the percent change in ZsGreen+ neutrophils over time. N=3-4 mice per group, representative of 2 independent experiments. Data are means ±SEM.

FIGS. 2A-2S overall show skull bone marrow supplies brain borders with myeloid cells. FIG. 2A is the experimental design for bone marrow egress experiments. The outer periosteal layer of the skull bone was thinned near the bone marrow sites and 1 μl of CXCR4 antagonist AMD3100 (1 μg/ml) or a vehicle was applied to five sites for 5 min. FIG. 2B is the quantification for flow cytometry analysis of IV CD45−Ly6C+ monocytes in cranial dura 1 day after AMD3100 administration. *P<0.05 (Student's t-test), n=3-4 mice per group, representative of 2 independent experiments. Data are means ±SEM. FIG. 2C is the quantification for flow cytometry analysis of IV neutrophils in cranial dura 1 day after AMD3100 administration. *P<0.05 (Student's t-test), n=3-4 mice per group, representative of 2 independent experiments. Data are means ±SEM. FIG. 2D is the quantification for flow cytometry analysis of IV CD45−Ly6C+ monocytes in control tissues 1 day after AMD3100 administration. NS=not significant (Two-way ANOVA with Sidak's post hoc test), n=3-4 mice per group, representative of 2 independent experiments. Data are means ±SEM. FIG. 2E is the quantification for flow cytometry analysis of IV neutrophils in control tissues 1 day after AMD3100 administration. NS=not significant (Two-way ANOVA with Sidak's post hoc test), n=3-4 mice per group, representative of 2 independent experiments. Data are means ±SEM. FIG. 2F is a schematic representation of the calvaria flap transplantation experiments. Images were taken on anesthetized mice by stereomicroscopy immediately after craniotomy (left), after the subsequent calvaria flap transplantation (middle), and 30 days after the transplantation (right). FIG. 2G shows representative images from 3 mice of GFP+ calvaria flaps 7 and 30 days after the transplantation. Inserts show GFP bone marrow 7 and 30 days after the transplantation. Dead bone marrow was replaced by non-GFP cells. Scale bar, 500 μm. FIG. 2H shows representative images from 4 mice of cranial dura 7 and 30 days after the transplantation showing GFP cells below the transfer site. Scale bar, 1000 μm. FIG. 2I is representative histograms of GFP and GFP (middle) and 30 days after the transplantation (right). FIG. 2G is representative images from 3 mice of GFP++++−+Ly6C+ monocytes and neutrophils across tissues. WT and UBC-GFP control mice without IV anti-CD45 antibody injection were used to assign gates. FIG. 2J is a quantification of flow cytometry analysis of IV CD45−GFP+Ly6C+ monocytes from calvaria flap transplanted mice, n=6-7 mice per group, representative of 2 independent experiments for day 7. Data are means ±SEM. FIG. 2K is a quantification of flow cytometry analysis of IV GFP+ neutrophils from calvaria flap transplanted mice, n=6-7 mice per group, representative of 2 independent experiments for day 7. Data are means ±SEM. FIG. 2L is a quantification of flow cytometry analysis of IV CD45−GFP+Ly6C+ monocytes from calvaria flap transplanted mice, n=6-7 mice per group, representative of 3 independent experiments for day 30. Data are means ±SEM. FIG. 2M is a quantification of flow cytometry analysis of IV GFP+ neutrophils from calvaria flap transplanted mice, n=6-7 mice per group, representative of 3 independent experiments for day 30. Data are means ±SEM. FIG. 2N is the experimental design of irradiation and bone marrow transplantation experiments with different shielding strategies. Four million GFP+ bone marrow cells were intravenously transplanted into head-shielded or body-shielded WT mice after split-dose 11 Gy irradiation. FIG. 2O is representative histograms of GFP− and GFP+Ly6C+ monocytes and neutrophils across tissues. Fully irradiated and non-irradiated mice transplanted with UBC-GFP cells without IV anti-CD45 antibody injection were used as control animals and to assign gates. FIG. 2P is a quantification of flow cytometry analysis for IV CD45−GFP+Ly6C+ monocytes in BMT experiments. NS=not significant, *P<0.05, **P<0.01 versus blood (one-way ANOVA with Dunnett's post hoc test), n=4 mice per tissue, representative of 2 independent experiments. Data are means ±SEM. BM, bone marrow. FIG. 2Q is a quantification of flow cytometry analysis for GFP+ neutrophils in BMT experiments. NS=not significant, *P<0.05, **P<0.01 versus blood (one-way ANOVA with Dunnett's post hoc test), n=4 mice per tissue, representative of 2 independent experiments. Data are means ±SEM. BM, bone marrow. FIG. 2R is a quantification of flow cytometry analysis for IV CD45−GFP+Ly6C+ monocytes in BMT experiments. NS=not significant, *P<0.05, **P<0.01 versus blood (one-way ANOVA with Dunnett's post hoc test), n=4 mice per tissue, representative of 2 independent experiments. Data are means ±SEM. BM, bone marrow. FIG. 2S is a quantification of flow cytometry analysis for IV GFP+ neutrophils in BMT experiments. NS=not significant, *P<0.05, **P<0.01 versus blood (one-way ANOVA with Dunnett's post hoc test), n=4 mice per tissue, representative of 2 independent experiments. Data are means ±SEM. BM, bone marrow. FIGS. 3A-3L overall show the inflamed CNS is infiltrated by blood and CNS-adjacent bone marrow-derived myeloid cells. FIG. 3A is the experimental design for EAE induction in parabiotic mice. Sixty days after pairing of WT and UBC-GFP mice EAE was induced in both mice by MOG35-55 immunization and the WT mice were analyzed 15 days post-induction. FIG. 3B is representative histograms of GFP− and GFP+Ly6C+ monocytes and neutrophils across tissues. WT and UBC-GFP control mice without IV anti-CD45 antibody injections were used to assign gates. FIG. 3C is a quantification of flow cytometry analysis of IV CD45−GFP+Ly6C+ monocytes in EAE-induced WT parabionts. NS=not significant, ***P<0.001 versus blood (one-way ANOVA with Dunnett's post hoc test), n=5 mice per tissue, representative of 2 independent experiments. Data are means ±SEM. FIG. 3D is a quantification of flow cytometry analysis of IV GFP+ neutrophils in EAE-induced WT parabionts. NS=not significant, ***P<0.001 versus blood (one-way ANOVA with Dunnett's post hoc test), n=5 mice per tissue, representative of 2 independent experiments. Data are means ±SEM. FIG. 3E shows representative images from 3 mice of cranial dura, spinal dura, and spinal cord sections of WT parabionts 15 days after the EAE induction. Only a minor portion of GFP+ cells are co-stained with GR1. Scale bars, (left) 2000 μm; (middle) 2000 μm; (right) 200 μm. FIG. 3F shows representative H & E and immunohistochemistry images from 5 mice of vascular channels found in vertebrae connecting bone marrow to underlying spinal dura. On the left, cells can be seen within channels connecting bone marrow and spinal dura. On the right, GR1+ infiltrates are closely associated with the CD31 vascular channel. Scale bars, (left) 100 μm; (right) 100 μm. FIG. 3G shows tSNE visualizations of color-coded scRNA-seq analysis based on cell types for CD45IV CD45GFP or GFP populations from the spinal cord of WT parabionts 15 days post EAE induction. N=3 pooled mice per sample. FIG. 3H shows dot plots demonstrating scaled gene expression and percentage of cells expressing these genes for cluster phenotyping markers. FIG. 3I shows cluster distributions detailing proportions of cell types in IV CD45CD45GFP and GFP samples from the scRNA-seq analysis. FIG. 3J shows the top 10 downregulated GO Biological Process terms describing differentially expressed genes between GFP μm. Numbers in bar graphs represent the number of differentially expressed genes belonging to that pathway. FIG. 3K shows the top 10 upregulated GO Biological Process terms describing differentially expressed genes between GFP μm. Numbers in bar graphs represent the number of differentially expressed genes belonging to that pathway. FIG. 3L is a volcano plot showing differentially expressed genes between GFP+ and GFP− monocytes from scRNA-seq analysis, inflammatory chemokines, and cytokines are highlighted.

FIGS. 4A-4P overall show CSF accesses skull bone marrow niches. FIG. 4A is a representative maximum intensity projection of a decalcified and cleared skull cap-dura whole mount 1 hour after an ICM injection of OVA-A594. Scale bar, 2 mm. FIG. 4B shows Z-sections through the dura, cortical skull bone, and trabecular bone marrow in a region of interest from the skull cap-dura whole mount. Arrowheads highlight perivascular OVA accumulation. Arrows denote OVA+ cells within the bone marrow cavity. Scale bars, 100 μm. FIG. 4C is schematics of the anatomy of skull bone marrow niches in the dorsal and basal skull. FIG. 4D shows sagittal sections through the dorsal and basal skull. Scale bars, 3 mm. FIG. 4E shows high-magnification images of tracer accumulation in the dorsal (left) and basal (right) bone marrow of the skull 1 hour after an ICM injection of OVA-A594. Scale bars, 50 μm. FIG. 4F is a representative two-photon image of skull bone marrow in a live mouse 30 minutes after administration of 70 kDa FITC-dextran (i.v.) and OVA-594 (ICM). Scale bar, 100 μm. FIG. 4G is a gating strategy and representative plots of ICM OVA-A488 labeling in bone marrow macrophages. FIG. 4H is a gating strategy and representative plots of ICM c-Kit-PE labeling in bone marrow HSCs. FIG. 4I is the experimental design for ICM c-Kit-PE. FIG. 4J is a graph of the percentage of macrophages positive for ICM OVA-A488 1 hour after injection. n=4 or 5 mice. Data are means ±s.e.m.; P values represent a one-way ANOVA with Tukey's post hoc test. FIG. 4K is a graph of the percentage of HSCs positive for ICM c-Kit-PE 1 hour after injection. n=4 or 5 mice. Data are means ±s.e.m.; P values represent a one-way ANOVA with Tukey's post hoc test. FIG. 4L is the experimental design for the IC c-Kit PE experiment. FIG. 4M is a graph of the percentage of macrophages positive for IC OVA-A594 1 hour after injection. n=4 mice. Data are means ±s.e.m.; P values represent a one-way ANOVA with Tukey's post hoc test. FIG. 4N is a graph of the percentage of HSCs positive for IC c-Kit-PE 1 hour after injection. n=4 mice. Data are means ±s.e.m.; P values represent a one-way ANOVA with Tukey's post hoc test. FIG. 4O is an experimental design for a c-Kit-PE experiment. FIG. 4P is a graph of the percentage of bone marrow HSCs that are positive for c-Kit-PE 1 hour after ICM injection in P7, P14, P21, adult (2-3 months old), and aged (20-24 months old) mice. n=4 mice. Data are means ±s.e.m.; P values represent a two-way ANOVA with Dunnett's post hoc test versus 2-3 months. BM, bone marrow.

FIGS. 5A-5J overall describe functional interactions between CSF and the skull bone marrow niche. FIG. 5A is a t-distributed stochastic neighbor embedding (t-SNE) visualizations of scRNA-seq from the dorsal skull and tibial bone marrow from 2-month-old mice colored by cell type. FIG. 5 is a t-distributed stochastic neighbor embedding (t-SNE) visualizations of scRNA-seq from the dorsal skull and tibial bone marrow from 2-month-old mice colored by sample. FIG. 3C is a chord plot detailing between CSF ligands identified by unlabeled LC-MS and receptors on skull bone marrow HSCs, macrophages, monocytes, and neutrophils identified by scRNA-seq. FIG. 5D is a Gene Ontology (GO) pathway analysis on receptor genes with at least one CSF ligand in monocytes. FIG. 5E is a Gene Ontology (GO) pathway analysis on receptor genes with at least one CSF ligand in neutrophils. FIG. 5F is representative immunohistochemistry of CD3+ and Ly6b+ cells at the superior sagittal sinus (S.S. Sinus) and transverse sinus (T. Sinus) of the dura mater, 24 hours after an ICM injection of 10 μg AMD3100 or aCSF. Scale bar, 200 μm. FIG. 5G is a flow cytometry gating strategy. FIG. 5H is a graph of the frequency of Ly6Chi monocyte after an ICM injection of 10 μg AMD3100 or aCSF. n=5 mice per group. Data are means ±s.e.m.; P values represent a two-sided Student's t-test. DCs, dendritic cells; NK, natural killer; DN, double negative. FIG. 5I is a graph of the frequency of neutrophils after an ICM injection of 10 μg AMD3100 or aCSF. n=5 mice per group. Data are means s.e.m.; P values represent a two-sided Student's t-test. DCs, dendritic cells; NK, natural killer; DN, double negative. FIG. 5J is a graph of the frequency of T cell proportions after an ICM injection of 10 μg AMD3100 or aCSF. n=5 mice per group. Data are means ±s.e.m.; P values represent a two-sided Student's t-test. DCs, dendritic cells; NK, natural killer; DN, double negative.

FIGS. 6A-6K overall describe that CSF-contained cues mobilize the skull bone marrow in response to CNS injury. FIG. 6A is the experimental design for spinal cord injury experiments. Spinal cord injury was performed in 2-month-old mice at T7 after laminectomy (sham group included laminectomy). EdU (10 mg kg-1) was injected 1 hour before collection. Three hours later, skull bone marrow was processed for flow cytometry. FIG. 6B is a gating strategy for MDPs, cMoPs, and actively proliferating Ki67+EdU+Ly6Chi monocytes. FIG. 6C is a schematic detailing the differentiation of the monocyte lineage from common myeloid progenitors (CMPs). FIG. 6D is a graph of the frequency of MDPs in the skull bone marrow of sham and spinal cord injury animals. n=5 mice per group. Data are means ±s.e.m.; P values represent a two-sided Student's t-test. FIG. 6E is a graph of the frequency of cMoPs in the skull bone marrow of sham and spinal cord injury animals. n=5 mice per group. Data are means ±s.e.m.; P values represent a two-sided Student's t-test. FIG. 6F is a graph of the frequency of Ly6Chi monocytes in the skull bone marrow of sham and spinal cord injury animals. n=5 mice per group. Data are means ±s.e.m.; P values represent a two-sided Student's t-test. FIG. 6G is a graph of the frequency of actively proliferating Ki67+EdU+Ly6Chi monocytes in the skull bone marrow of sham and spinal cord injury animals. n=5 mice per group. Data are means ±s.e.m.; P values represent a two-sided Student's t-test. FIG. 6H is the experimental design for CSF transfer experiments after spinal cord injury. CSF was collected from the cisterna magna of sham or spinal cord injury mice 3 hours after injury, and 10 μl was transferred to naive mice via ICM injection. After 6 hours, dural meninges from mice with CSF transferred were collected. FIG. 6I is a flow cytometry gating strategy for dural Ly6Chi monocytes after transfer of sham or spinal cord injury CSF. FIG. 6J is a graph of the absolute numbers of monocytes in the dura after transfer of CSF from sham and spinal cord injury mice. n=5 mice per group. Data are means ±s.e.m.; P values represent a two-sided Student's t-test. SCI, spinal cord injury. FIG. 6K is a graph of the frequency of monocytes in the dura after transfer of CSF from sham and spinal cord injury mice. n=5 mice per group. Data are means ±s.e.m.; P values represent a two-sided Student's t-test. SCI, spinal cord injury.

FIGS. 7A-7C overall describe the characterization of stem and immune cell populations in the basal skull marrow. FIG. 7A is a flow cytometry gating strategy for major immune populations in the skull bone marrow. FIG. 7B is a graph of the absolute numbers of CD45+ cells in the dorsal and basal skull marrow. n=3 mice. Mean±SEM. FIG. 7C is a graph of the relative frequencies of immune populations in the dorsal skull, basal skull, and tibial bone marrow. n=3 mice. Mean±SEM.

FIGS. 8A-8K describe the characterization of differences between the skull and tibial marrow populations. FIG. 8A is a dot plot demonstrating scaled gene expression and percentage of cells expressing genes for cluster phenotyping markers for bone marrow cell types from scRNA-seq analysis. FIG. 8B is an analysis of cluster proportions in the skull and tibial bone marrow. FIG. 8C is a volcano plot of differentially expressed genes in neutrophils. Magenta dots represent upregulated transcripts, while cyan dots represent downregulated transcripts in skull populations compared to the tibia. y-axes represent adjusted log 2 p-value for cluster changes between skull and tibia. The dotted line represents an adjusted p-value of 0.05 (general linear mixed model with Benjamini-Hochberg correction). FIG. 8D is a volcano plot of differentially expressed genes in monocytes. Magenta dots represent upregulated transcripts, while cyan dots represent downregulated transcripts in skull populations compared to the tibia. y-axes represent adjusted log 2 p-value for cluster changes between skull and tibia. The dotted line represents an adjusted p-value of 0.05 (general linear mixed model with Benjamini-Hochberg correction). FIG. 8E is a volcano plot of differentially expressed genes in macrophages. Magenta dots represent upregulated transcripts, while cyan dots represent downregulated transcripts in skull populations compared to the tibia. y-axes represent adjusted log 2 p-value for cluster changes between skull and tibia. The dotted line represents an adjusted p-value of 0.05 (general linear mixed model with Benjamini-Hochberg correction). FIG. 8F is a volcano plot of differentially expressed genes in HSCs. Magenta dots represent upregulated transcripts, while cyan dots represent downregulated transcripts in skull populations compared to the tibia. y-axes represent adjusted log 2 p-value for cluster changes between skull and tibia. The dotted line represents an adjusted p-value of 0.05 (general linear mixed model with Benjamini-Hochberg correction). FIG. 8G is a graph of the top 10 downregulated gene ontology pathways in skull vs. tibia for differentially expressed genes in neutrophils. FIG. 8H is a graph of the top 10 downregulated gene ontology pathways in skull vs. tibia for differentially expressed genes in monocytes. FIG. 8I is a graph of the top 10 downregulated gene ontology pathways in skull vs. tibia for differentially expressed genes in macrophages. FIG. 8J is a graph of the top 10 downregulated gene ontology pathways in skull vs. tibia for differentially expressed genes in HSCs. FIG. 8K is a dot plot of receptor expression in skull bone marrow cells, scaled by gene expression and percentage of cells expressing the gene, showing expression of receptors for which there is a cognate CSF ligand.

FIGS. 9A-9G describe the effects of AMD3100 on the immune cell composition of the dura and bone marrow. FIG. 9A is the experimental design for injections for skull bone marrow egress experiments. AMD3100 (10 μg) or artificial cerebrospinal fluid (aCSF) was injected intra-cisterna magna (i.c.m.), and mice were left for 24 hours. The following day, tissues were processed for immunolabeling or flow cytometry. FIG. 9B shows representative images of Ly6b+ cells and CD3+ cells in non-sinus regions of the dura. Scale bar: 200 μm. FIG. 9C is a regional analysis of Ly6b+ myeloid in the dura following AMD3100 administration. n=3 mice per group. Data are means ±SEM, p values represent two-way ANOVA with Sidak's post hoc test. FIG. 9C is a regional analysis of CD3+ cells in the dura following AMD3100 administration. n=3 mice per group. Data are means ±SEM, p values represent two-way ANOVA with Sidak's post hoc test. FIG. 9E is a flow cytometry gating strategy for neutrophils, Ly6Chi monocytes, macrophages, and T cells in the bone marrow following AMD3100 administration. FIG. 9F is a group of graphs of the relative numbers of neutrophils, Ly6Chi monocytes, macrophages, and T cells in the skull bone marrow 24 hours following i.c.m. AMD3100 administration. n=5 mice per group. Data are means ±SEM, p values represent a two-sided Student's t-test. FIG. 9G is a group of graphs of the relative numbers of neutrophils, Ly6Chi monocytes, macrophages, and T cells in the tibial bone marrow 24 hours following i.c.m. AMD3100 administration. n=5 mice per group. Data are means ±SEM, p values represent a two-sided Student's t-test.

FIGS. 10A-10D demonstrate that laminectomy does not affect CSF efflux to skull bone marrow. FIG. 10A shows representative flow plots of macrophages in the skull and tibia bone marrow with either sham surgery or laminectomy. Laminectomy, or sham surgery, was performed on mice and 3 hours later OVA-488 was injected into the cisterna magna. Tissues were collected 1 hour later for flow cytometry. FIG. 10B is a graph of the quantification of i.c.m. injected OVA uptake in macrophages following sham surgery or laminectomy. n=5 mice per group. Data are means ±SEM, p values represent a two-way ANOVA. FIG. 10C shows representative flow plots of i.c.m. anti-c-Kit-PE staining in LSKs in the skull and tibia bone marrow with either sham surgery or laminectomy. FIG. 10D is a graph of the quantification of i.c.m. injected cKit-PE uptake in LSKs following sham surgery or laminectomy. n=5 mice per group. Data are means ±SEM, p values represent a two-way ANOVA with Sidak's post hoc test.

FIGS. 11A-11F describe the effects of spinal cord injury on vertebral bone marrow. FIG. 11A is a schematic of an experimental paradigm for spinal cord injury experiments. Spinal cord injury (SCI) or laminectomy (sham) was performed, and at 3 hours post-injury vertebra adjacent to the site of injury were processed for flow cytometry. FIG. 11B is a graph of the relative numbers of monocyte dendritic precursors (MDPs) in the vertebral bone marrow. n=5 mice per group. p values represent a two-sided Student's t-test. FIG. 11C is a graph of the relative numbers of common monocyte progenitors (cMoPs) in the vertebral bone marrow. n=5 mice per group. p values represent a two-sided Student's t-test. FIG. 11D is a graph of the relative numbers of Ly6Chi monocytes in the vertebral bone marrow. n=5 mice per group. p values represent a two-sided Student's t-test. FIG. 11E is a graph of the relative numbers of actively proliferating (Ki-67+, EdU+) monocytes in the vertebral bone marrow. n=5 mice per group. p values represent a two-sided Student's t-test. FIG. 11F is a graph of the multiplexed measurement of cytokines and chemokines in the CSF of sham and SCI mice using Luminex. n=5. p values represent two-sided t-tests with Holm-Sidak's multiplicity adjustment. Data are means ±SEM.

FIGS. 12A-12E demonstrate that intracisternal injection of LPS enhances hematopoiesis in skull bone marrow and triggers myeloid egress to the dura. FIG. 12A is a group of 4 representative flow plots of neutrophils in the dura in aCSF and LPS-treated mice. LPS (1.25 μg, 4 μL) was injected into the skull bone marrow. After 24 hours, skullcaps and dura were processed for flow cytometry. FIG. 12B is a group of representative flow plots of Ly6Chi monocytes in the dura in aCSF and LPS-treated mice. FIG. 12C is a group of representative flow plots of LSKs in the skull BM of aCSF and LPS-treated mice. FIG. 12D is a graph of the quantification of the proportion of CD45+ immune cells and the absolute number of neutrophils and Ly6Chi monocytes in the dura of aCSF and LPS-treated mice. n=5 mice per group. Mean±SEM. p values represent a two-sided Student's t-test. FIG. 12E is a graph of the quantification of the proportion of live cells and the proportion of actively proliferating Ki67+ stem/progenitor (LSK, MDP, cMoP, GMP, GP) and myeloid (neutrophils, Ly6Chi monocytes) cells in the skull bone marrow of aCSF and LPS-treated mice. n=5 mice per group. Mean±SEM. P values represent a two-sided Student's t-test.

FIG. 13 is a summary schematic for the proposed mechanism in CNS. Brain interstitial fluid and cerebrospinal fluid can efflux to skull bone marrow during healthy conditions. During CNS insults—for example, pathogenic infections or spinal cord injury-CSF-derived cues can promote skull bone marrow hematopoiesis and egress of myeloid cells to underlying dura. HSC; hematopoietic stem cell, CSF; cerebrospinal fluid, ISF; interstitial fluid, SAS; subarachnoid space, BM; bone marrow, CNS, central nervous system.

FIGS. 14A-14R summarize the additional analysis of steady-state parabiosis experiments and monocyte progenitor cells. FIG. 14A is a graph of the quantification of flow cytometry analysis from parabiosis experiments of total GFP+CD45+ cells in the WT blood after 60 days of parabiosis. FIG. 14B is a graph of the quantification of flow cytometry analysis from parabiosis experiments of IV CD45−GFP+Ly6C− monocytes across tissues. NS=not significant, ***p<0.001 versus blood (One-way ANOVA with Dunnett's post hoc test), n=8 mice/tissue. FIG. 14C is a graph of the quantification of flow cytometry analysis from parabiosis experiments of IV B cells across tissues. NS=not significant, ***p<0.001 versus blood (One-way ANOVA with Dunnett's post hoc test), n=8 mice/tissue. FIG. 14D is a graph of the quantification of flow cytometry analysis from parabiosis experiments of IV CD45−GFP+ macrophages across tissues. NS=not significant, **p<0.01, ***p<0.001 versus Spleen (One-way ANOVA with Dunnett's post hoc test), n=8 mice/tissue. FIG. 14E is a graph of the quantification of flow cytometry analysis from parabiosis experiments of IV cDC1s across tissues. NS=not significant, **p<0.01, ***p<0.001 versus Spleen (One-way ANOVA with Dunnett's post hoc test), n=8 mice/tissue. FIG. 14F is a graph of the quantification of flow cytometry analysis from parabiosis experiments of IV cDC2s across tissues. NS=not significant, **p<0.01, ***p<0.001 versus Spleen (One-way ANOVA with Dunnett's post hoc test), n=8 mice/tissue. FIG. 14G is a graph of the quantification of HSC progenitor cells across different hematopoietic stem cell niches in WT parabiont. FIG. 14H is a graph of the quantification of IV CD45−Ly6C+ monocyte chimerism in the blood and cranial dura in CD45.1-CD45.2 parabiotic mice 60 days after the surgery.*p<0.05 (Student's t-test), n=4 mice/tissue. FIG. 14I is a graph of the quantification of IV neutrophil chimerism in the blood and cranial dura in CD45.1-CD45.2 parabiotic mice 60 days after the surgery. *p<0.05 (Student's t-test), n=4 mice/tissue. FIG. 14J is an experimental design of in-vivo EdU labeling. 10 mg/kg of EdU was injected i.p. daily for two days and analyzed by flow cytometry 24 hours after the last injection. FIG. K is a representative gating strategy of monocyte progenitor cells in the bone marrow and dura. FIG. 14L is a graph of the quantification of the total number of IV CD45−cMoPs in the skull bone marrow and cranial dura. *p<0.05, **p<0.01, ***p<0.001 (Student's t-test), n=4 mice/tissue. FIG. 14M is a graph of the quantification of the percent of live cells of IV CD45−cMoPs in the skull bone marrow and cranial dura. *p<0.05, **p<0.01, ***p<0.001 (Student's t-test), n=4 mice/tissue. FIG. 14N is a graph of the quantification of the total number of IV CD45−MDPs in the skull bone marrow and cranial dura. *p<0.05, **p<0.01, ***p<0.001 (Student's t-test), n=4 mice/tissue. FIG. 14O is a graph of the quantification of the percent of live cells of IV CD45−MDPs in the skull bone marrow and cranial dura. *p<0.05, **p<0.01, ***p<0.001 (Student's t-test), n=4 mice/tissue. FIG. 14P shows representative histograms of EdU+ monocyte progenitor cells in the skull bone marrow and dura. FIG. 14Q is a graph of the quantification of EdU+ monocyte progenitor cells in the skull bone marrow and dura. n=5 mice/group. FIG. 14R shows representative histograms of Ki-67+ monocyte progenitor cells in the skull bone marrow and dura. BM, bone marrow. FIG. 14R is a graph of the quantification of Ki-67+ monocyte progenitor cells in the skull bone marrow and dura. n=4 mice/group. BM, bone marrow.

FIGS. 15A-15M describe the additional analysis of proliferative capacity, fate mapping, and migratory potential of meningeal myeloid cells. FIG. 15A is a representative gating strategy for myeloid fate mapping using LysM-CreERT2::ZsGreen reporter mice. FIG. 15B is a graph of the quantification of ZsGreen+ neutrophils across tissues. Points represent mean±SEM. n=¾ mice/group. FIG. 15C is a graph of the quantification of Ly6C+ monocytes across tissues. Points represent mean±SEM. n=¾ mice/group. FIG. 15D is a graph of the quantification of Ly6C-Ly6G double negative cells across tissues. Points represent mean±SEM. n=¾ mice/group. FIG. 15E shows representative histograms IV CD45−EdU+Ly6C+ monocytes and neutrophils in the blood, skull bone marrow, and cranial dura. FIG. 15E is a graph of the quantification of IV CD45−EdU+Ly6C+ monocytes in the blood, skull bone marrow, and cranial dura. NS=not significant, *p<0.05, **p<0.01, ***p<0.001 (One-way ANOVA with Tukey's post hoc test), n=5 mice/tissue. FIG. 15F shows representative histograms and quantification of IV EdU+ neutrophils in the blood, skull bone marrow, and cranial dura. NS=not significant, *p<0.05, **p<0.01, ***p<0.001 (One-way ANOVA with Tukey's post hoc test), n=5 mice/tissue. FIG. 15H shows representative histograms of IV CD45−Ki-67+Ly6C+ monocytes and neutrophils in the blood, skull bone marrow, and cranial dura. FIG. 15I is a graph of the quantification of IV CD45−Ki-67+Ly6C+ monocytes in the blood, skull bone marrow, and cranial dura. NS=not significant, **p<0.01, (One-way ANOVA with Tukey's post hoc test), n=5 mice/tissue. FIG. 15J is a graph of the quantification of IV Ki-67+ neutrophils in the blood, skull bone marrow, and cranial dura. NS=not significant, **p<0.01, (One-way ANOVA with Tukey's post hoc test), n=5 mice/tissue. FIG. 15K is the experimental design for photoconversion of KikGR mice. Skull bone marrow was photoconverted for two minutes daily for two days and analyzed 24 hours after the last photoconversion. FIG. 15L is a gating strategy for KikGreen and KikRed Ly6C+ monocytes and neutrophils across tissues. FIG. 15M is a graph of the quantification of KikGreen and KikRed Ly6C+ monocytes and neutrophils across tissues. NS=not significant, *p<0.05, **p<0.01, ***p<0.001 (Two-way ANOVA with Sidak's post hoc test), n=3 mice/group. BM, bone marrow. dCLN, deep cervical lymph node.

FIGS. 16A-16N demonstrate that injections in the skull bone marrow result in dural immediate leakage. FIG. 16A is an image of a dissected skull flap from a mouse that received two skull bone marrow injections of 6 μl of Evans Blue. Red arrows represent injection sites. FIG. 16B is a dissected skull flap following bone marrow injections viewed from a ventral perspective. Injections performed on the dorsal side did not cause damage to the ventral side of the skull, confirming the efficacy of the injection and ruling out that the subsequent leakage of the blue dye was due to the needle penetrating the skull too deep. Red asterisks in the magnification represent what appear to be natural porosities of the skull where the cranial dura is more tightly attached to the skull bones anatomically arranged to “host” the blood sinuses. FIG. 16C is an image wherein Evans blue injected in the skull bone marrow leaks to the dura and is efficiently uptaken by surrounding lymphatics. Blue arrows highlight the dye signal. FIG. 16D is an image wherein Evans blue injected in the skull bone marrow leaks to the dura and is efficiently uptaken by surrounding lymphatics. Blue arrows highlight the dye signal. FIG. 16E is an image of a coronal section of a decalcified skull with the dura mater left attached from a Prox1creERT2::Ai14 (tdTomato) mouse. The red signal (highlighted by white arrows) represents dural lymphatics and appears localized below the sutures of the cranial bones. FIG. 16F is an image of a coronal section of a decalcified skull with the dura mater left attached from a Prox1creERT2::Ai14 (tdTomato) mouse. The red signal (highlighted by white arrows) represents dural lymphatics and appears localized below the sutures of the cranial bones. FIG. 16G is an immunohistochemistry image of coronal sections of decalcified skulls with the dura mater left attached from WT mice that underwent skull bone marrow injections of fresh UBC-GFP cells flushed from a GFP+ femur. Images show a successful injection of GFP+ cells in the skull bone marrow and leakage of GFP+ cells along the superior sagittal sinus and in the stroma of the dura. FIG. 16H is another immunohistochemistry image of coronal sections of decalcified skulls with the dura mater left attached from WT mice that underwent skull bone marrow injections of fresh UBC-GFP cells flushed from a GFP+ femur. FIG. 16I is yet another immunohistochemistry image of coronal sections of decalcified skulls with the dura mater left attached from WT mice that underwent skull bone marrow injections of fresh UBC-GFP cells flushed from a GFP+ femur. FIG. 16J is an immunohistochemistry image of dura mater from a mouse injected in the skull bone marrow with a CD45−Pe antibody. The antibody extensively leaked in the dura mater labeling immune cells along the sinus and in the dural stroma. FIG. 16K is an immunohistochemistry image of dura mater from a mouse injected in the skull bone marrow with a CD45−Pe antibody. The antibody extensively leaked in the dura mater labeling immune cells along the sinus and in the dural stroma. FIG. 16L is an injection strategy of green and red cell dyes inside the skull and tibia bone marrow. 2 μl of CellTracker Far Red was injected into skull bone marrow in 2 different spots and 3 μl CellTracker Green was injected in tibia bone marrow. Tissues were analyzed 24 hours after injection. FIG. 16M is a gating strategy for CellTracker-labeled monocytes in tissues of interest. FIG. 16N is a graph of the quantification of flow cytometry analysis for CellTracker Green and CellTracker Far Red labeled monocytes. NS=not significant, ***p<0.001 (Two-way ANOVA followed with Bonferroni's Post-Hoc test). BM, bone marrow. SSS, superior sagittal sinus.

FIGS. 17A-17N summarize the additional analysis of calvaria flap transplantation experiments. FIG. 17A is a graph of the quantification of DAPI coverage of cranial flaps seven and 30 days after transplantation. NS=not significant, *p<0.05 (One-way ANOVA with Tukey's post hoc test), n=3 mice/group. FIG. 17B is a graph of the quantification of GFP signal in cranial flaps seven and 30 days after the transplantation. NS=not significant (Student's t-test), n=3 mice/group. FIG. 17C is a graph of the quantification of CD31 coverage of underlying cranial dura seven and 30 days after the calvaria flap transplantation. NS=not significant, *p<0.05, **p<0.01 (One-way ANOVA with Tukey's post hoc test), n=3 mice/group. FIG. 17D is a graph of the quantification of GFP signal in underlying cranial dura seven and 30 days after the calvaria flap transplantation. NS=not significant (Student's t-test), n=3 mice/group. FIG. 17E is a representative image of cleared skull-dura whole-mount containing a transplanted calvarium bone flap. FIG. 17F is an image wherein clusters of dural CCR2+ cells are co-labeled with the GFP+ signal. FIG. 17G is an image showing Iba1+GFP+ dura macrophage and CD31+GFP+ meningeal vasculature. FIG. 17H is a set of graphs of the quantification of flow cytometry analysis of different IV CD45− immune populations seven days after the transplantation. n= 6/7 mice/tissue. FIG. 17I is a set of graphs of the quantification of flow cytometry analysis of different IV CD45− immune populations 30 days after the transplantation. n= 6/7 mice/tissue. FIG. 17J is an image wherein vascularized skull channels connecting the skull and dura are detected 30 days after transplantation. White arrowheads indicate an ossified skull channel labeled with OsteoSense. GFP+ vasculature was detected inside the channels anastomosing with dura vasculature FIG. 17K is another image wherein vascularized skull channels connecting the skull and dura are detected 30 days after transplantation. White arrowheads indicate an ossified skull channel labeled with OsteoSense. GFP+ vasculature was detected inside the channels anastomosing with dura vasculature. FIG. 17L is a representative image of microglia in transplanted and naive mice 30 days after the transplantation. FIG. 17M is a Sholl analysis of microglia in transplanted and naive mice 30 days after the transplantation. NS=not significant (Two-way ANOVA with Geisser-Greenhouse correction) n=4 mice/group. FIG. 17N is a weight graph of mice that underwent calvaria flap transplantation. Mice were followed up to 30 days after transplantation. BM, bone marrow. dCLN, deep cervical lymph node.

FIGS. 18A-18E describe ligand-receptor inferences for homeostatic dural monocyte and neutrophil recruitment. FIG. 18A is a uMAP visualization of color-coded scRNA-seq of whole dura populations based on cell types, n=10 total pooled mice per sample, from two independent experiments. FIG. 18B is a list of the top chemokine-chemokine receptor pairings predicted by RNA magnet between all dural cell populations as the ligand source and monocytes or neutrophils as the receptor source. FIG. 18C is a proteome profiler array of chemokine expression in dura homogenates of WT mice. n=2 samples, 3 mice/sample. FIG. 18D is a uMAP visualization of expression for selected ligand (purple) and receptor (green) pairings for chemokine receptor signaling in monocytes and neutrophils. FIG. 18E is a graphical representation of likely ligand-receptor pairings for monocyte and neutrophil chemoattraction based on dural protein expression of the ligand and monocyte and neutrophil expression of the chemokine receptor.

FIGS. 19A-19I summarize additional analysis for EAE parabiosis and the relationship of myeloid cells with meningeal lymphatics. FIG. 19A is the experimental design for EAE induction in parabiotic mice. 60 days after pairing of WT and UBC-GFP mice EAE was induced in both mice by MOG35-55 immunization and the WT mice were analyzed 15 days post-induction. FIG. 19B is a representative gating strategy for flow cytometry of IV CD45− immune cells from the spinal cord post EAE induction. FIG. 19C is a graph of the quantification of IV CD45− labeling of CD45hi immune populations in blood and tissues. FIG. 19D is a graph of the quantification of flow cytometry analysis of IV CD45−GFP+Ly6C− monocytes in EAE-induced WT parabionts. NS=not significant, **p<0.01 versus blood (One-way ANOVA with Dunnett's post hoc test), n=5 mice/tissue. FIG. 19E is a graph of the quantification of flow cytometry analysis of IV CD4 T cells in EAE-induced WT parabionts. NS=not significant, **p<0.01 versus blood (One-way ANOVA with Dunnett's post hoc test), n=5 mice/tissue. FIG. 19F is a graph of the quantification of flow cytometry analysis of IV microglia and macrophages in EAE-induced WT parabionts. NS=not significant, **p<0.01 versus blood (One-way ANOVA with Dunnett's post hoc test), n=5 mice/tissue. FIG. 19G is an immunohistochemistry image wherein GR1+ cell association with LYVE1+ lymphatic vasculature 15 days after EAE induction was observed. The orthogonal view shows a GR1+GFP− cell within the lymphatic vessel lumen. FIG. 19H is a graph of the quantification of GFP+ cell colocalization with LYVE1+ lymphatic structures in the cranial dura. BM, bone marrow. FIG. 19I is a graph of the quantification of GR1+ cell colocalization with LYVE1+ lymphatic structures in the cranial dura. BM, bone marrow.

FIGS. 20A-20O summarize the additional analysis of CNS and peripheral injury models. FIG. 20A is an experimental design for spinal cord injury in CD45.1 and CD45.2 parabiotic mice. 60 days after pairing spinal cord injury was performed in both mice by injury at Th9 level with fine forceps. CD45.1 and CD45.2 mice were analyzed three days post-injury. FIG. 20B is a representative gating strategy for flow cytometry of IV CD45− immune cells from the spinal cord following spinal cord injury. FIG. 20C is a representative plot demonstrating gating for CD45.1 and CD45.2 monocytes in a CD45.1 host. FIG. 20D is a graph of the quantification of flow cytometry analysis for donor-derived IV CD45−Ly6C+ monocytes in host tissues. NS=not significant, *p<0.05, **p<0.01, ***p<0.001 versus blood (One-way ANOVA with Dunnett's post hoc test), n=6 mice/tissue. FIG. 20E is a graph of the quantification of flow cytometry analysis for donor-derived IV neutrophils in host tissues. NS=not significant, *p<0.05, **p<0.01, ***p<0.001 versus blood (One-way ANOVA with Dunnett's post hoc test), n=6 mice/tissue. FIG. 20F is a graph of the quantification of flow cytometry analysis for donor-derived IV CD4 T cells in host tissues. NS=not significant, *p<0.05, **p<0.01, ***p<0.001 versus blood (One-way ANOVA with Dunnett's post hoc test), n=6 mice/tissue. FIG. 20G is a graph of the quantification of flow cytometry analysis for donor-derived IV Ly6c− monocytes in host tissues. NS=not significant, *p<0.05, **p<0.01, ***p<0.001 versus blood (One-way ANOVA with Dunnett's post hoc test), n=6 mice/tissue. FIG. 20H is a graph of the quantification of flow cytometry analysis for IV CD45− microglia and macrophages in host tissues. FIG. 20I is an experimental design for optic nerve crush in WT and UBC-GFP parabiotic mice. 60 days after pairing unilateral optic nerve crush was performed using fine forceps in the WT mice and analyzed one day post-injury. FIG. 20J is a representative gating strategy for flow cytometry of IV CD45− immune cells from the optic nerve following optic nerve crush. FIG. 20K is a graph of the quantification of flow cytometry analysis for IV CD45−GFP+Ly6C+ monocytes in WT tissues. *p<0.05, ***p<0.001 (Student's t-test), n=4 mice/tissue. FIG. 20K is a graph of the quantification of flow cytometry analysis for IV GFP+ neutrophils in WT tissues. *p<0.05, ***p<0.001 (Student's t-test), n=4 mice/tissue. FIG. 20M is an experimental design for skin injury in CD45.1 and CD45.2 parabiotic mice. 60 days after pairing skin injury was performed by ear puncture in both mice and CD45.1 and CD45.2 mice were analyzed one day post-injury. FIG. 20N is a representative gating strategy for flow cytometry of IV CD45− immune cells from the ear skin following skin injury. FIG. 20O is a graph of the quantification of flow cytometry analysis for donor derived IV CD45−Ly6C+ monocytes in host tissues. NS=not significant (Student's t-test), n=5 mice/tissue. BM, bone marrow. FIG. 20O is a graph of the quantification of flow cytometry analysis for donor derived IV neutrophils in host tissues. NS=not significant (Student's t-test), n=5 mice/tissue. BM, bone marrow.

FIGS. 21A-21C establish that the spinal cord is surrounded by the vertebral bone marrow. FIG. 21A is a schematic representation of the vertebra at the lumbar level. FIG. 21B is a representative IHC staining of the whole spine cross section from a naive mouse stained with DAPI, GR1, and CD31. GR1+ bone marrow is seen in the body, as well as lamina and processes of the vertebrae. A vascular channel connecting bone marrow to the spinal dura is seen. FIG. 21C is a representative H & E staining of the spinal cross section from naive mice containing vertebrae bone marrow and spinal cord. Note that, enlarged artificial subdural space is the result of paraffin processing, not seen in cryosectioned samples in FIG. 21B. BM, bone marrow.

FIGS. 22A-22L summarize additional analysis for EAE and spinal cord injury scRNA-seq. FIG. 22A is an experimental design for EAE induction in WT and UBC-GFP parabiotic mice. 60 days after pairing of WT and UBC-GFP mice EAE was induced in both mice by MOG35-55 immunization and the WT mice were analyzed 15 days post-induction. FIG. 22B is a gating strategy for scRNA-seq analysis of IV CD45−CD45hi GFP− or GFP+ populations from the spinal cord of WT parabionts 15 days post EAE induction. FIG. 22C is a graph of the quantification of flow cytometry analysis for IV CD45−Ly6C+ monocyte, neutrophil, CD4 T cell, and B cell blood chimerism in WT EAE parabionts that were sorted for scRNA-seq analysis. FIG. 22D is an experimental design for spinal cord injury in WT and UBC-GFP parabiotic mice. 60 days after pairing of WT and UBC-GFP mice spinal cord injury was performed in both mice by injury at Th9 level with fine forceps and the WT mice were analyzed three days post-injury. FIG. 22E is a gating strategy for scRNA-seq analysis of IV CD45−CD45hi GFP− or GFP+ populations from the spinal cord of WT parabionts three days post spinal cord injury. FIG. 22F is a graph of the quantification of flow cytometry analysis for IV CD45−Ly6C+ monocyte, neutrophil, CD4 T cell, and B cell blood chimerism in WT spinal cord injury parabionts that were sorted for scRNA-seq analysis. FIG. 22G is a tSNE visualization of color-coded scRNA-seq analysis based on cell types for IV CD45−CD45hi GFP− or GFP+ populations from the spinal cord of WT parabionts three days post spinal cord injury. n=4 pooled mice per sample. FIG. 22H is a Dot plot demonstrating scaled gene expression and percentage of cells expressing these genes for cluster phenotyping markers. FIG. 22I is a graph of cluster distributions detailing proportions of cell types in GFP− and GFP+ samples from scRNA-seq analysis. FIG. 22J is a graph of the top 10 downregulated GO Biological Process terms describing differentially expressed genes between GFP+ and GFP monocytes from scRNA-seq analysis. FIG. 22K is a graph of the top 10 upregulated GO Biological Process terms describing differentially expressed genes between GFP+ and GFP monocytes from scRNA-seq analysis. FIG. 22L is a volcano plot showing differentially expressed genes between GFP+ and GFP− monocytes from scRNA-seq analysis, inflammatory chemokines and cytokines are highlighted.

### DETAILED DESCRIPTION OF THE INVENTION

Recent works described the existence of direct ossified vascular channels connecting skull bone marrow to meninges, capable of dispersing neutrophils during inflammation.

We hypothesized that these connections may also allow homeostatic myeloid trafficking between adjacent meningeal and bone marrow niches. Here, we demonstrate that CNS-associated bone marrow niches in the skull and vertebrae are myeloid reservoirs for the meninges and CNS parenchyma.

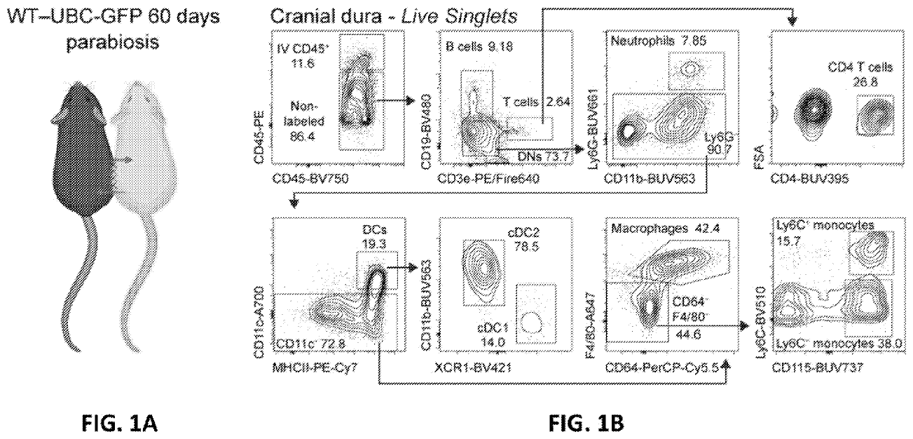

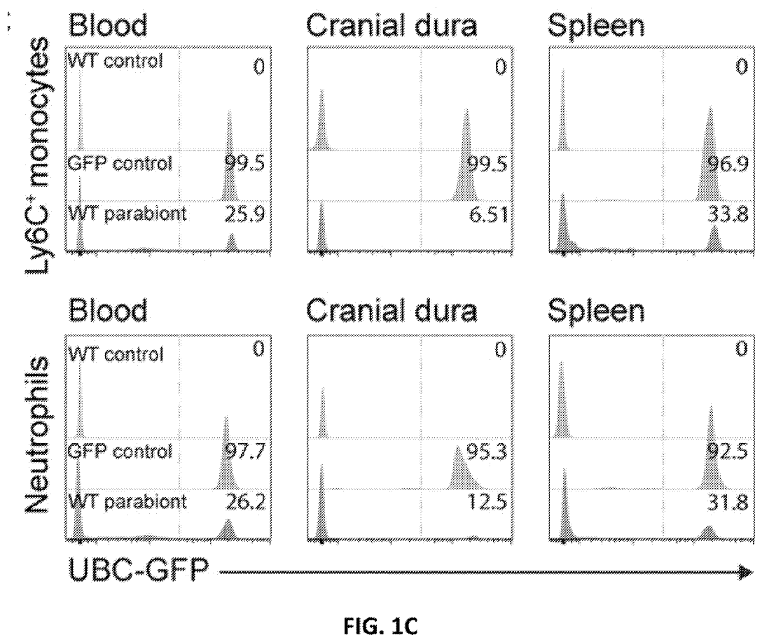

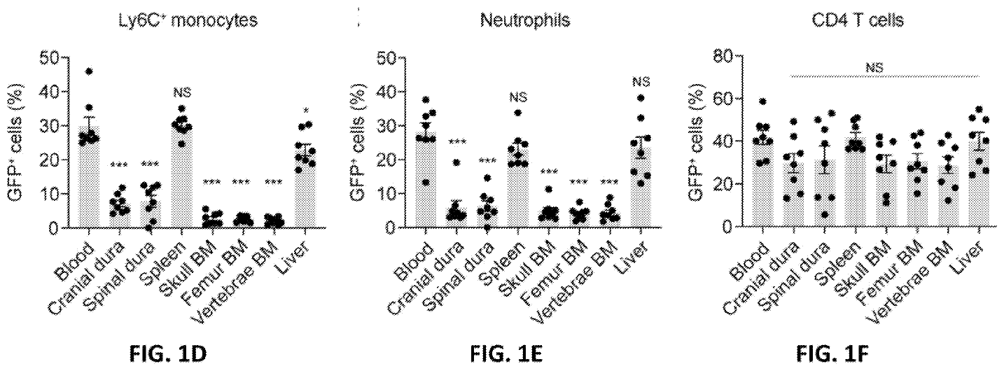

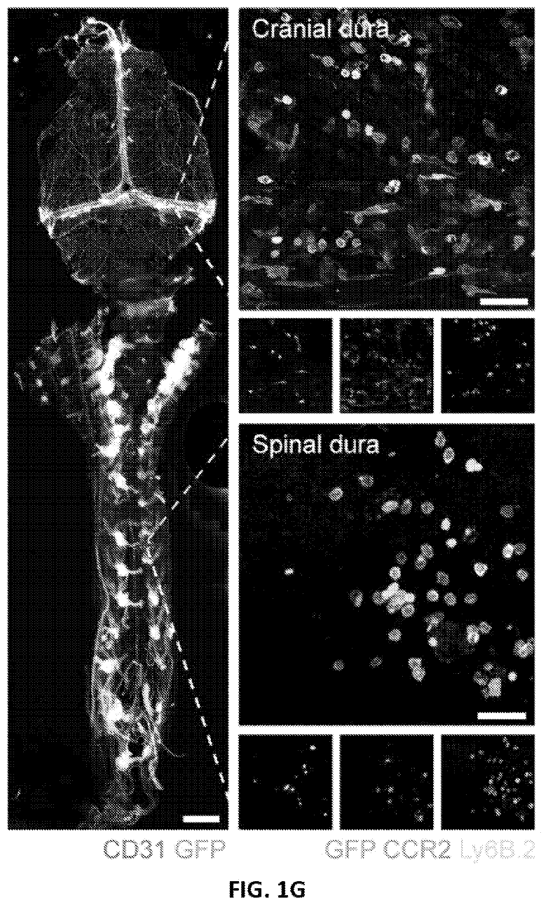

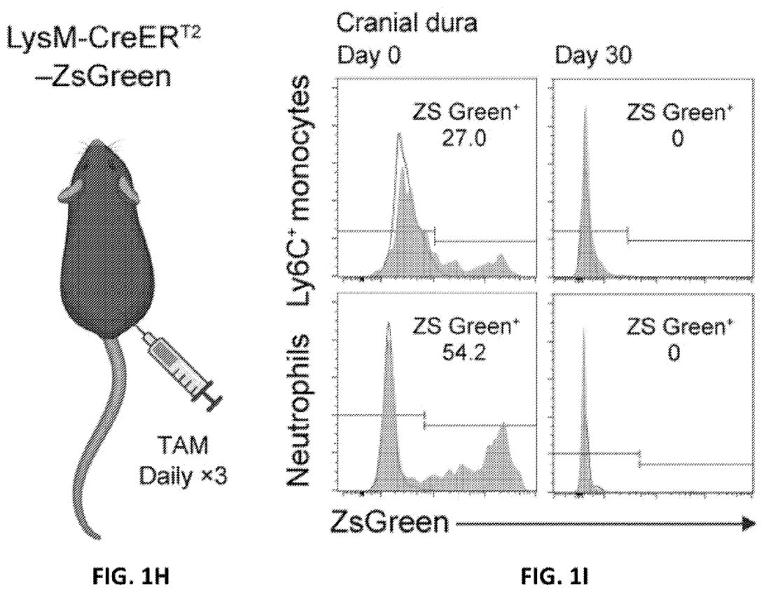

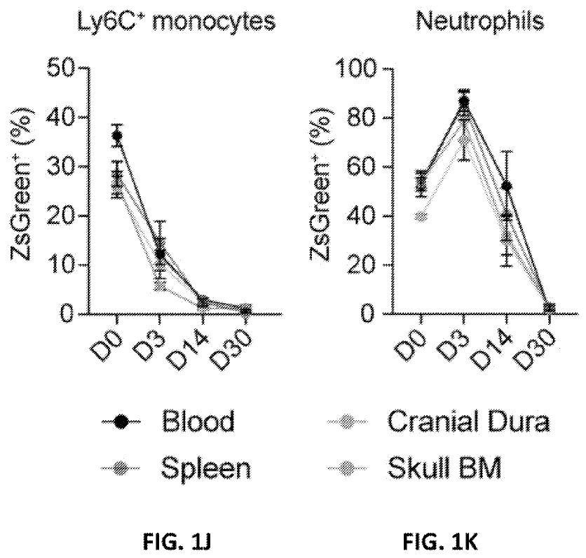

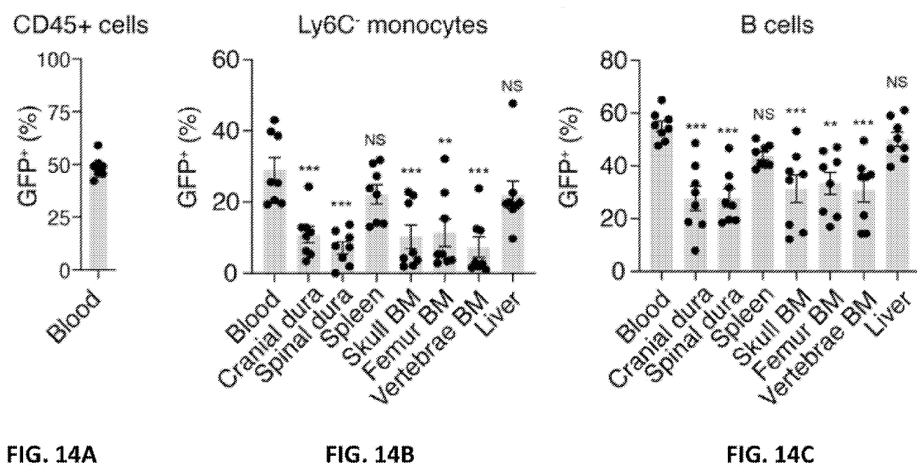

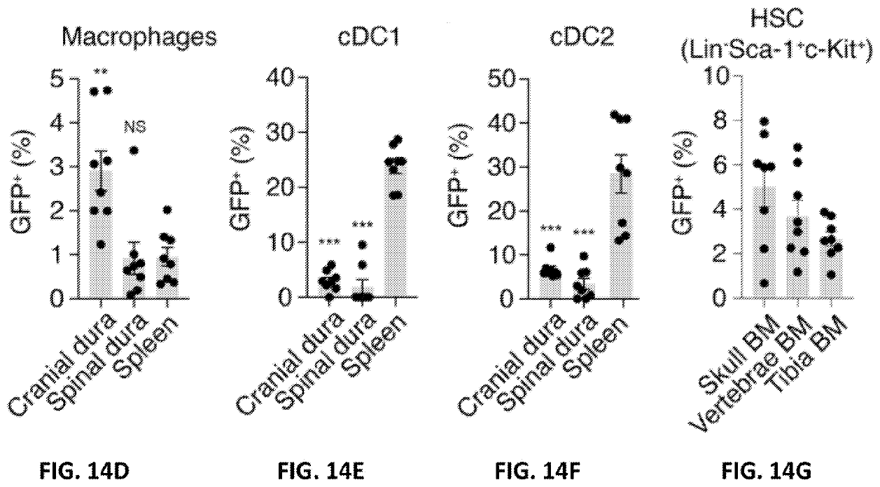

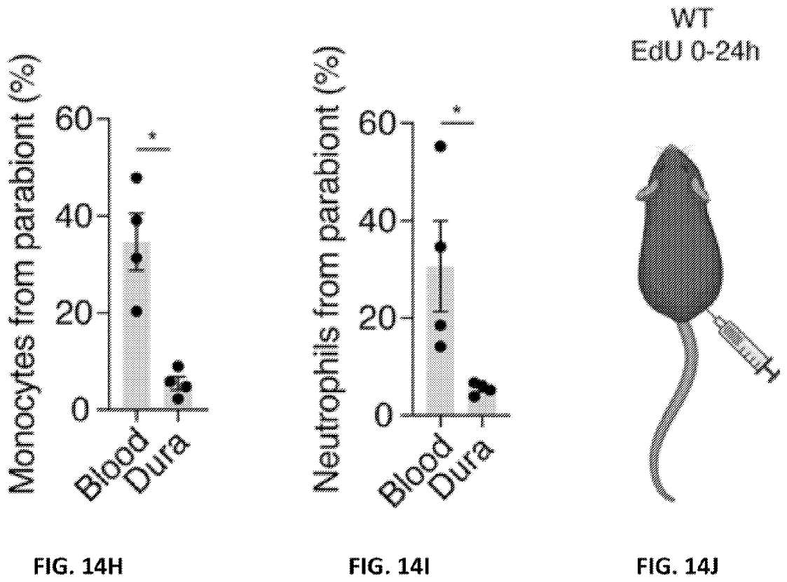

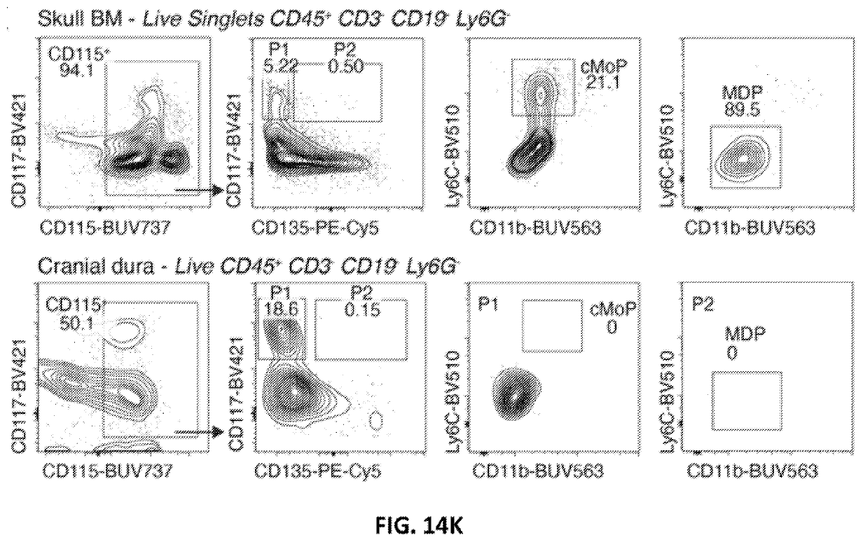

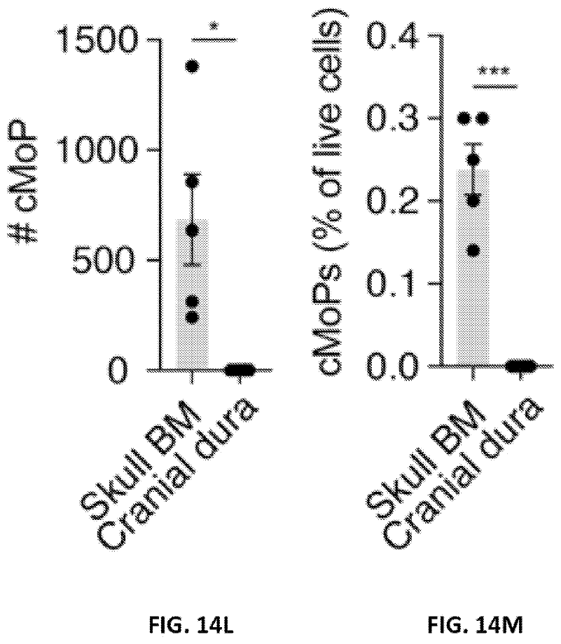

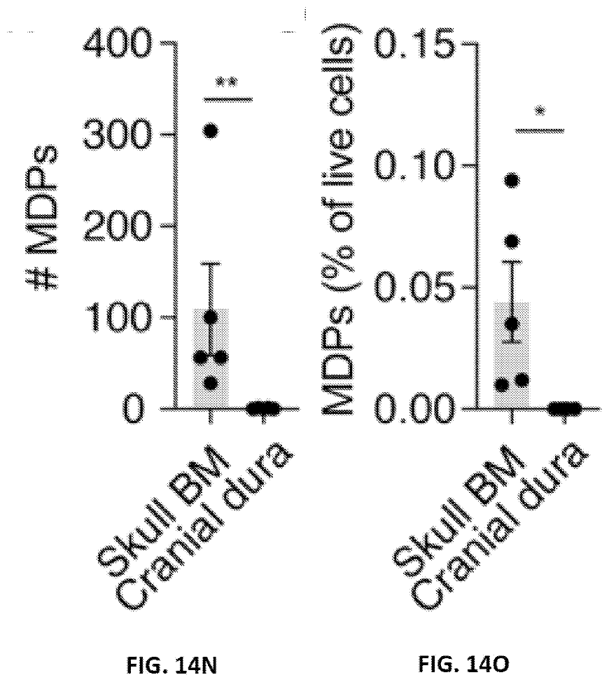

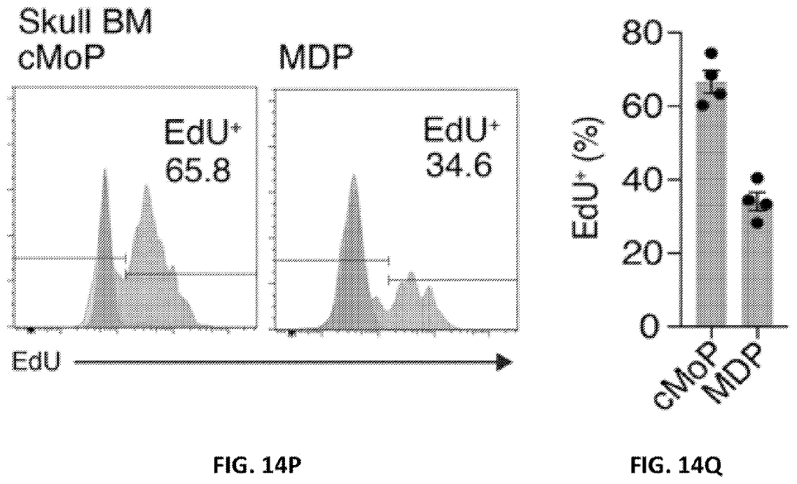

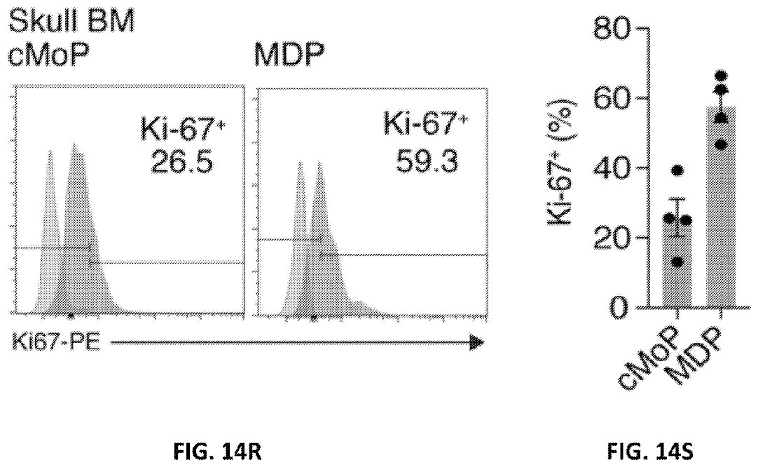

Under homeostasis, these bone marrow pools supply the brain and spinal dural meninges with monocytes and neutrophils via direct dural-bone marrow connections. Following injury and neuroinflammation, these cells can mobilize to infiltrate the CNS parenchyma, and display distinct phenotypes from their blood-derived counterparts. In an attempt to understand the origin of border myeloid cells under homeostasis, we parabiotically joined the circulations of UBC-GFP and WT mice and performed flow cytometric analyses of the blood, brain-associated tissues (cranial dura mater and spinal dura mater), hematopoietic organs (skull bone marrow, vertebrae bone marrow, and femur bone marrow), and peripheral control tissues (spleen and liver) after 60 days of pairing (FIGS. 1A, 1B, and 1C). To ensure our analysis only examined true parenchymal populations and not blood contaminants, mice received intravenous anti-CD45 antibodies two minutes prior to euthanasia, labeling blood and vascular-associated leukocytes that were subsequently excluded in our gating strategy (FIG. 1B). Analysis of WT parabionts demonstrated that while blood chimerism for total CD45+ cells reached a 50:50 ratio (FIG. 14A), neither neutrophils nor monocytes ever reached perfect chimerism in the blood and after two months a GFP:WT cell ratio of ˜30:70 was achieved (FIGS. 1D and 1E). The lower chimerism of these myeloid cells is likely due to the combination of a short half-life of these cells and active myelopoiesis in the bone marrow.

Despite imperfect blood chimerism of Ly6C+ monocytes and neutrophils, GFP+ cell proportions detected in the cranial dura and spinal dura were significantly lower than that of blood and that would be expected given a blood origin (FIGS. 1D and 1E). These data suggested a substantial pool of monocytes and neutrophils located along the brain borders that had not originated from the UBC-GFP parabiont. By comparison, the myeloid chimerism displayed in other peripheral tissues was more akin to the proportions present in the blood (FIGS. 1D and 1E). Cranial and spinal dural CD4 T cells from the UBC-GFP parabiont were also not significantly different from the blood chimerism, suggesting a blood origin as described previously (FIG. 1F). Further, GFP+Ly6c− monocytes, and B cells displayed different levels of chimerism in distinct tissues, specifically GFP+ B cells appeared to be less represented at the brain borders, also suggesting a non-blood origin (FIGS. 14B and 14C). Additional components of the dural myeloid compartment including macrophages, cDC1s, and cDC2s also displayed a low proportion of GFP+ cells compared to the non-border associated spleen pool (FIGS. 14D, 14E, and 14F). Analysis of several bone marrow niches confirmed that the majority of detected hematopoietic progenitors Lin-/SCA-1/C-Kit (LSK) were GFP− cells, as expected from an active

As described in the examples below, it was demonstrated that the steady-state myeloid niche located at the CNS borders in mice consists of a substantial pool of monocytes and neutrophils that are continuously replenished from bone-marrow niches in the adjacent skull or vertebrae. Our model of calvaria bone-flap transplantation-used here as a proof-of-concept prototype- and various irradiation regimes demonstrate that skull bone marrow can supply the adjacent meningeal tissue with myelomonocytic cells without using the blood route. Moreover, our results indicate that these bone-marrow-derived monocytes can differentiate into meningeal macrophages. This revealed a myeloid niche that is open to homeostatic peripheral inputs that, in contradiction to the classic Van Furth dogma, does not use the blood as a major route.

It is tempting to speculate that channels aligned between the bone marrow and the dura might, under homeostatic conditions, represent a principal route for the migration of monocytes and neutrophils. In our skull transplantation model, we detected GFP+ vascular elements sprouting from the bone-marrow niche and extending toward the dural parenchyma. It is therefore reasonable to conceive of a scenario wherein myelomonocytic cells crawl abluminally along these vessels using the vasculature as a scaffold, as is the case of neuroblasts during CNS development, glioma tumor spread in the brain parenchyma, or T cells finding their zone in the spleen.

Beyond homeostatic trafficking, we demonstrated that in the context of either injury or autoimmune neuroinflammation, monocytes can invade the brain and spinal cord parenchyma without the need for blood, and thus the breakdown of the blood-brain barrier. In this sense, the brain seems to behave as a unique organ, in that it is able to hold a privileged dialogue with its surrounding tissues. Indeed, we envisage these monocytes and neutrophils as a myeloid reservoir situated at the borders of the CNS under homeostatic conditions and ready to leap to its defense when needed, thereby complementing blood-borne inflammatory cells in supporting the CNS. Whether dural-derived chemokines differentially favor migration from local bone marrow over blood, or indeed whether the two routes use different recruitment factors entirely remains to be clarified. Intriguingly, this could allow for the manipulation of specific subsets of myeloid cells, with potentially unique phenotypes. From our scRNA-seq analysis, it appears that infiltrating Ly6c+ cells from the blood and the bone marrow may have distinct roles in EAE pathology, potentially through differences in immune cell recruitment or exacerbation of inflammatory events, though the exact participation remains to be seen. It will also be intriguing to further explore the kinetics of blood- and CNS-associated bone marrow-derived myeloid infiltration during EAE development, to explore whether one population represents the “first responders” to CNS disruption.

The skull and vertebral bone-marrow-derived meningeal myeloid reservoir described here reshape our interpretation of the neuroimmunological events occurring at the CNS borders under both physiological and pathological conditions. It appears likely that this reservoir of immune cells may also be of relevance in other scenarios of brain pathology, such as neurodegeneration, brain tumors, CNS or meningeal infection, and aging, or in the context of microglial replacement under specific ablative regimens.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs the transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit the translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein-encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein are within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by IIe, Leu by IIe, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/I). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged | K R H |
| (Basic): | |
| Negatively Charged | D E |
| (Acidic): | |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| | Leu, Val, Met, Ala, |
| Ile (I) | Phe, Ile, Val, Met, |
| | Ala, |
| Leu (L) | Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| | Ile, Leu, Met, Phe, |
| Val (V) | Ala |

Exemplary nucleic acids which may be introduced to a host cell include, for example, DNA sequences or genes from another species, or even genes or sequences which originate with or are present in the same species but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tₘ of specific internal domains of the siRNA, siRNA length, position of the target sequence within the CDS (coding region), and nucleotide content of the 3′ overhangs.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Also provided is a process of treating, preventing, or reversing a CNS injury or disorder in a subject in need by administration of a therapeutically effective amount of meningeal immune cells, so as to prevent, reduce, or reverse the CNS injury or disorder.

Methods described herein are generally performed on a subject in need thereof. A subject in need of the therapeutic methods described herein can be a subject having, diagnosed with, suspected of having, or at risk for developing a CNS injury or disorder. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of meningeal immune cells is, for example, an amount that would cause the desired therapeutic effect in a subject while minimizing undesired side effects. In various embodiments, an effective amount of meningeal immune cells described herein can substantially inhibit a CNS injury or disorder, slow the progress of a CNS injury or disorder, or limit the development of a CNS injury or disorder.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of meningeal immune cells can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to prevent, reduce, or reverse a CNS injury or disorder.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of meningeal immune cells can occur as a single event or over a time course of treatment. For example, meningeal immune cells can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for prevention, reduction, or reversal of a CNS injury or disorder.

Meningeal immune cells can be administered simultaneously or sequentially with another agent, such as an antibiotic, an anti-inflammatory, or another agent. For example, meningeal immune cells can be administered simultaneously with another agent, such as an antibiotic or an anti-inflammatory. Simultaneous administration can occur through the administration of separate compositions, each containing one or more of meningeal immune cells, an antibiotic, an anti-inflammatory, or another agent. Simultaneous administration can occur through the administration of one composition containing two or more of meningeal immune cells, an antibiotic, an anti-inflammatory, or another agent. Meningeal immune cells can be administered sequentially with an antibiotic, an anti-inflammatory, or another agent. For example, meningeal immune cells can be administered before or after the administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

### Screening

Also provided are methods for screening. The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8A to about 15A.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to compositions containing meningeal immune cells as described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, and sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

To demonstrate that CNS-associated bone marrow niches in the skull and vertebrae are myeloid reservoirs for the meninges and CNS parenchyma, the following experiments were conducted.

### Mice

Mice were housed in the animal facility 4 to 5 animals per cage in a temperature and humidity-controlled environment with a 12-hour light-dark cycle. Mice were provided with rodent chow and water ad libitum. All mice were housed for habituation for at least 1 week before the start of the experimentation. Unless specified otherwise, mice were 2-3 months of age at the beginning of the experiments. The following strains were used: C57BL/6 (WT; JAX 000664), C57BL/6-Tg(UBC-GFP)30Scha/J(UBC-GFP; JAX 004353), B6.SJL-Ptprca Pepcb/BoyJ (CD45.1; JAX 002014), Lyz2 tm1 (CREER-T2)Grtn/J (LysM-CREER-T2; JAX 031674), B6.Cg-Gt(ROSA)26Sor tm6(CAG-ZsGreen1)Hze/J (Zsgreen, JAX 007906), Prox1-CREER-T2 (JAX 022075), B6.Cg-Gt(ROSA)26Sor tm14(CAG-tdTomato)Hze/J (Ai14; JAX 007914),) Tg(CAG-KikGR)33Hadj/J (KikGR, JAX 013753). Mice were bred in-house or purchased from the Jackson Laboratory. All experiments were approved by the Institutional Animal Care and Use Committee of the University of Virginia and/or Washington University in St. Louis. Experiments were only performed in institutions for which experimental approval was granted.

### Parabiosis

Mice were anesthetized with an intraperitoneal injection of ketamine (100 mg per kg)-xylazine (10 mg per kg) cocktail. Fur was shaved on the corresponding lateral aspect of each mouse and cleaned with alternating Betadine solution and alcohol swabs. After muscles were fully relaxed, a matching skin incision from the olecranon to the knee joint was made on each side of the incision, the subcutaneous fascia was bluntly dissected to create 0.5 cm of free skin. To reduce direct pulling on the skin sutures, the olecranon and knee were sutured together by an absorbable 5-0 Vicryl suture. After pressing together, the dermis of the parabiotic partners, skin incisions were closed with a 5-0 Nylon suture. Post-surgery, systemic analgesics (2.5 mg per kg ketoprofen) and antibiotics (2.5 mg per kg Baytril, Bayer) were administered. One single parabiotic pair was housed per cage, wet food was provided, and mice were monitored daily for pain and distress. Unless specified otherwise, all parabiotic pairs were sacrificed after 60 days, disjoined, and separately perfused.

### Tissue Collection and Processing for Immunohistochemistry

Mice were sacrificed using a lethal dose of anesthetics (Euthasol, 10% v/v) via IP injection and transcardially perfused with 0.025% heparin in PBS solution. Cranial meninges and brain were harvested by immediate decapitation posterior to the occipital bone; skin, mandible, and muscles were removed, and the skull was fixed in 4% paraformaldehyde (PFA) at 4° C. for 24 h. After 24 hours meninges were peeled from the skull cup and placed in PBS with 0.25% sodium azide at 4° C. until used. Brains were fixed for additional 24 hours in PFA at 4° C., transferred in PBS with 30% sucrose until fully immersed and then embedded in OCT compound (Fisher), frozen rapidly over dry ice, and stored at −20° C. until use. Forty-micrometer-thick sections (coronal) were sliced on a cryostat (Leica). Calvarium and femur were fixed in 4% PFA at 4° C. for 24 hours, transferred in decalcification solution (PBS with 0.1 M of EDTA) at agitation for 4-6 days at 37° C. with daily change of the solution, and then transferred in PBS with 30% sucrose overnight. Calvarium and femur were then embedded in OCT, frozen rapidly over dry ice and stored at −20° C. until use. Thirty-micrometer-thick sections (coronal or sagittal) were sliced onto gelatin-coated slides on a cryostat (Leica). Vertebral laminae were cut on both sides, vertebral foramen was exposed, and the spinal cord was dissected using fine forceps and fixed in 4% PFA at 4° C. for 48 hours. Meninges were cut along the ventral side and whole-mount meninges were peeled from the spinal cord and placed in PBS with 0.25% sodium azide at 4° C. until used. The spinal cord was transferred in PBS with 30% sucrose overnight, embedded in OCT, frozen rapidly over dry ice and stored at −20° C. until use. Twenty five-micrometer-thick sections were sliced onto gelatin-coated slides on a cryostat (Leica). Liver, lung, and lymph nodes were harvested using forceps, placed in 4% paraformaldehyde (PFA) overnight, transferred in PBS containing 30% sucrose overnight, then embedded in OCT and frozen rapidly over dry ice and stored at −20° C. until use. Twenty five-micrometer-thick sections were sliced onto gelatin-coated slides on a cryostat (Leica). For immunofluorescence staining, tissues were rinsed in PBS and washed with PBS with 0.2% Triton X-100 for 10 min, followed by incubation in blocking and permeabilization buffer containing PBS with 0.2% Triton X-100 and 2% normal chicken serum for one hour at room temperature with gentle agitation. If applicable, endogenous biotin was blocked using a streptavidin/biotin blocking kit (Vector Laboratories SP-2002) following the manufacturer's instructions. To quench autofluorescence of femur and skull bone marrow, Trueblack (Biotium, 23007) was used following the manufacturer's instructions. Sections were then incubated with primary antibodies in the blocking and permeabilization buffer for 24 hours at 4° C. with gentle agitation, washed three times with PBS with 0.2% Triton X-100 and incubated with secondary antibodies in the blocking and permeabilization buffer for 2 hours at room temperature. Slices were washed three times with PBS containing 0.2% Triton X-100, and nuclei were counterstained with DAPI. Free floating sections were mounted on Superfrost Plus slides and coverslipped with Aqua-Mount (Lerner) or ProLong Gold Antifade Mountant (Molecular Probes) and glass coverslips. Gelatin-coated slides were mounted with Aqua-Mount (Lerner) and glass coverslips.

For H & E staining, formalin fixed spines were embedded in paraffin and ten-micrometer-thick sections were cut on a microtome (Leica), mounted onto slides, deparaffinized, and stained with Hematoxylin and Eosin Stain Kit (Vector Laboratories H-3502) according to the manufacturer's instructions.

For cleared skull-dura whole-mount imaging, the vDISCO tissue clearing and processing protocols were used as described. Briefly, after perfusion, the skull with attached meninges was dissected, and placed in PBS 4% PFA overnight, followed by decolorization solution for 24 hours. After washing with PBS, samples were placed in decalcification solution (10 wt/Vol % EDTA (Carl Roth, 1702922685) in 0.1 M PBS) for two days at room temperature. Samples were then permeabilized, stained, and cleared using the vDISCO protocol with passive tissue immersion as described. Cleared samples were immersed in Ethyl cinnamate (Sigma, W243019) and placed in a chambered cover-glass (Thermo, 155360) for imaging.

### Confocal Microscopy and Image Analysis

Tissues slides were stored at 4° C. for no more than 1 week until images were acquired using confocal microscopy (Leica, TCS SP8 or Leica, Stellaris) with 10×(0.4 NA), 20×(0.75 NA), and 40×(1.3 NA) objectives (Leica) and widefield microscopy (Olympus Slideview VS200) with 10×(0.4 NA), 20×(0.8 NA), and 40×(0.95 NA) objectives (Olympus) and a Hamamatsu ORCA Flash 4.0 V3 digital camera. Quantitative analysis of acquired images was performed using the Fiji package for ImageJ. Five-fifteen representative images were acquired per sample and the results were averaged to generate the value utilized for a single mouse.

### Single-Cell Isolations

Mice were injected with a lethal dose of Euthasol (10% vol/vol) and transcardially perfused with ice-cold PBS with 0.025% heparin. Blood was collected before perfusion from the retroorbital sinus. Blood was centrifuged at 420 g for four minutes. Red blood cell lysis was then performed by resuspension in 1 ml of ACK lysis buffer (Quality Biological) for 10 minutes at room temperature, and 2 ml of PBS was added to inactivate ACK buffer. After centrifugation at 420 g for 4 minutes samples were resuspended in FACS buffer (PBS with 2% BSA) and kept at 4° C. until use. Tissues were harvested as described above. Briefly, dura and lymph nodes were digested for 15 min, and lung, liver, and skin for 30 min, at 37° C. in pre-warmed digestion buffer (DMEM with 1 mg/ml Collagenase VIII (Sigma Aldrich) supplemented with 0.5 mg/ml DNase I (Sigma-Aldrich), 2% FBS). After digestion tissues underwent several steps of mechanical homogenization followed by filtration through a 70-μm cell strainer. Enzymes were inactivated with DMEM with 10% FBS and cells were then centrifuged at 420 g for 4 min and washed in FACS buffer. Single-cell suspensions were kept in FACS buffer on ice until use. After dissecting the meninges, spinal cord and brains were harvested, followed by digestion step for 30 min in pre-warmed digestion buffer, triturated with a 10-ml serological pipette, digested for an additional 20 min and again triturated with a 5-ml serological pipette. Cells were passed through a 70-μm cell strainer, enzymes were inactivated by adding an equal volume of DMEM with 10% FBS, and samples were centrifuged at 420 g for four minutes. Myelin was removed by adding a 1:1 ratio of 22% BSA in PBS to DMEM and samples were centrifuged at 1000 g for 10 min, with no brake. The upper myelin-containing layer and underlying BSA was aspirated and the single-cell suspension was kept in FACS buffer until use. For the isolation of skull bone marrow, meninges were peeled from the skull cup as described above. The calvarium was then cut into small pieces using sterile scissors and mechanically dissociated in FACS buffer with a pestle, followed by a filtration step through a 70-μm cell strainer. After centrifugation at 420 g for 4 min, red blood cells were removed by adding 1 ml of ACK lysis buffer for 2 min at room temperature, centrifugation at 420 g for 4 min, and the cell pellet was resuspended in FACS buffer until use. Femur bone marrow was harvested by dissecting the left femur, proximal and distal epiphysis were cut using sterile scissors, and using a 23 G needle filled with PBS with 2% BSA bone marrow was flushed out from the femur onto a 70-μm cell strainer and gently pressed. After centrifugation, red blood cells were lysed with 1 ml ACK lysis buffer for 2 min, centrifuged at 420 g for 4 min, and resuspended in ice-cold FACS buffer until use.

### Flow Cytometry and Fluorescence-Activated Cell Sorting

Single-cell suspensions were washed and resuspended in FACS buffer. FC receptors were blocked for five minutes on ice using an anti-CD16/CD32 antibody cocktail (FC block; 2 μg/ml, Biolegend), followed by a 10-min incubation at room temperature with antibody cocktails for surface staining. The list of flow antibodies is provided in table S5. To determine cell viability, Zombie Aqua or Zombie NIR fixable Viability kit was used following the manufacturer's instructions. Samples were acquired with a Gallios flow cytometer (Beckman Coulter) or an Aurora spectral flow cytometer (Cytek). Data were analyzed using FlowJo version 10. For FACS a BD FACSAria II (BD Biosciences) was used.

### IV Immune Labeling

To label intravascular leukocytes, mice were retro-orbitally injected with 2 μg of PE-conjugated anti-CD45 antibody (Biolegend, clone 30-F11) under isoflurane anesthesia 2 min before euthanasia. In all instances, proper IV administration was confirmed by complete labeling of blood leukocytes.

### Tamoxifen-Induced Cre Recombination

To induce Cre recombination in LysmCreERT2 mice, a 20 mg/ml stock solution of tamoxifen (Sigma Aldrich) was prepared by dissolving in corn oil at 37° C. for 24 hours followed by sterile filtration. Transgenic mice were injected IP daily with 100 μl of tamoxifen at eight weeks of age for three consecutive days. Mice were then sacrificed at 0, 3, 14, or 30 days after the final tamoxifen injection.

### Intracalvaria and Intratibial Bone Marrow Injection

All procedures were performed on mice anesthetized with a ketamine (100 mg per kg)-xylazine (10 mg per kg) mixture. Body temperature was maintained throughout the surgery by placing mice on a heating pad at 37° C. while restrained in a stereotaxic apparatus. The head of the mouse was shaved, a skin midline incision was made, and the skull was exposed. Injections were performed similarly as described previously. Briefly, an electric drill was used to gently erode the compact bone covering bone marrow niches on occipital and frontal bones until a thin layer of periosteum was left. A Hamilton syringe complemented with a 34 G-blunt needle-RN 0.37 PT3 was used to manually poke the final hole for the injection. All injections were done manually. Every injection consisted of a maximum volume of 2 μl. A single injection took around 45 s. After the injection, the needle was maintained in place for 15 s in order to prevent excessive leakage of the injected solution.

Several injections were performed per mouse for a total final volume injected that ranged between 4 and 10-μl. Several types of injections were performed: (a) UBC-GFP progenitor cells freshly flushed from the femur (roughly 10,000 cells injected per mouse); (b) Evans Blue; (c) CD45-PE antibody (Biolegend, clone 30-F11); and (d) CellTracker Deep red (C34565, Molecular Probes). The Hamilton was filled directly with the concentrated antibody solution from the Biolegend/Molecular Probes vials. For intratibial bone marrow injection, a skin incision was made below the knee, and using a 30 G needle bone was perforated between tuberosity and crest of the tibia. Injection of 3 μl of CellTracker CMFDA (green) (C7025 Molecular Probes) dye was performed manually over 30 s. After the procedure, the skin was sutured, and mice were gently placed on a heating pad and monitored until awake. Once awake, mice were injected with Ketoprofen and antibiotics (enrofloxacin, 2.5 mg per kg). After the bone marrow injections, mice were sacrificed 2 or 24 hours later.

Skull bone marrow delivery of AMD3100 was performed as previously described. Briefly, mice were anesthetized with a ketamine (100 mg per kg)-xylazine (10 kg per kg) mixture. The head of the mouse was shaved, a skin midline incision was made and the skull was exposed. Using an electrical drill, the outer periosteal layer was thinned on top of skull bone marrow near the bregma and lambda in five spots, without damaging the bone marrow. One microliter of 1 mg/ml AMD3100 (ab120718, Abcam) or vehicle was applied on each spot for 5 min. The skin was sutured and mice were sacrificed 24 hours later.

### Calvarium Bone Flap Transplantation

Mice were anesthetized with a ketamine (100 mg per kg)-xylazine (10 mg per kg) mixture. Body temperature was maintained throughout the surgery by placing mice on a heating pad at 37° C. The heads of WT mice were shaved, skin midline incisions were made, and the skull was exposed. Using an electrical drill, a 4×6 mm cranial window was made on parietal and interparietal bones. During bone drilling, periodic pauses were made to cool the bone with 0.9% sodium chloride solution. After the cranial window was detached from surrounding bones, craniotomy was performed with the help of fine forceps, extra caution was made not to damage the underlying dura mater. Craniotomy was performed with abundant 0.9% sodium chloride solution bathing the brain. If dura mater, brain, or sinuses were damaged during the craniotomy, mice were sacrificed and discarded. In parallel, a sex-matched UBC-GFP mouse, whose skull bone marrow was used as a donor of the calvarium, was euthanized, decapitated, and a piece of calvarium comparable to the window drilled in the host mouse was obtained. Connective tissue and pericranium layers were left attached on the bone flap to increase the survival of the transplanted bone. Before the transplantation, meninges were peeled from the donor skull. The donor piece of skull containing the bone marrow was carefully placed within the surgical window created in the host mouse. Then, the donor skull was fixed to the host skull using cyanoacrylate glue and a trimmed 10-0 nylon suture needle. The skin was sutured, and mice were kept on a heating pad until they awoke. Mice were observed daily and injected with Ketoprofen (2.5 mg per kg) and antibiotics (2.5 mg per kg Baytril, Bayer) once per day for the first five days after the transplantation day.

### Experimental Autoimmune Encephalomyelitis (EAE)

For active induction of EAE in parabiotic pairs, both mice joined in parabiosis were immunized by subcutaneous injection of 200 μg of MOG35-55 (CSBio) in complete Freund's Adjuvant (Sigma Aldrich) with 4 mg/ml of Mycobacterium tuberculosis (H37Ra)(BD Biosciences). Mice received 200 ng of pertussis toxin (List Biological Laboratories) IP on day 0 (immunization day) and day 1 (24 hours after immunization).

### Spinal Cord Injury

Mice were anesthetized with a ketamine (100 mg per kg)-xylazine (10 mg per kg) mixture. The skin over the upper thoracic area was shaved and cleaned with betadine and alcohol. A 15-mm midline skin incision was made, and connective tissue and muscles were bluntly dissected. After a Th9 laminectomy, the spinal cord was crushed with fine forceps. After the injury, muscles and skin were sutured separately and allowed to recover from anesthesia on heating pads. Once awake mice were injected daily intraperitoneally with buprenorphine (0.05 mg per kg) and Baytril (Enrofloxacin, 2.5 mg per kg) for three consecutive days. Seventy-two hours after surgery mice were sacrificed for further analysis.

### Skin Injury

Mice in parabiosis were briefly anesthetized by placing them in an isoflurane induction chamber for three minutes, then a fast ear punch was performed on the left ear of each parabiont (WT and UBC-GFP mouse). The ear puncher was cleaned with a solution of 70% ethanol between each couple of parabionts. After the punch, mice were injected intraperitoneally with Ketoprofen analgesia (2.5 mg per kg) and placed back in their cages to wake. Mice were sacrificed 24 hours following skin injury.

### Optic Nerve Injury

Mice in parabiosis were anesthetized by placing them in an isoflurane induction chamber for three minutes. The soft tissue around the eye was dissected to expose the optic nerve. The optic nerve was crushed with an N5 self-closing forceps. Mice were injected intraperitoneally with Ketoprofen analgesia (2.5 mg per kg), put back in their cages, and allowed to recover on a heating pad. Mice were sacrificed 24 hours following optic nerve injury.

### Irradiation and Bone Marrow Transplantation

All mice received split-dose irradiation with the first dose of 550 rad (5.5 Gy) followed by another dose of 550 rad (5.5 Gy) 6-7 hours later. For full-body irradiation mice were irradiated without anesthetics. For head shielding, mice were anesthetized with a ketamine (100 mg per kg)-xylazine (10 mg per kg) mixture. Mice heads were shielded using a 1-inch-thick lead shield (1-inch Lead vial Shield, 50 ml, Pinestar Technology), exposing the body below the neck. Alternatively, for body shielding, mice were inserted into a lead shield exposing only the head. All mice were injected IV with 4×106 (100 μl in PBS) freshly isolated bone marrow cells from UBC-GFP donors 6 hours after the last irradiation. Mice were analyzed 4 weeks after the bone marrow transplantation.

### EdU and Ki67 Staining for Proliferation Analysis

Mice received two intraperitoneal injections of 10 mg per kg EdU 24 hours apart and were sacrificed 24 hours after the final injection. After the generation of single-cell suspensions for flow cytometry and surface staining as described earlier, fixation, permeabilization, and EdU staining were performed following the manufacturer's instruction (Click-iT™ Plus EdU Alexa Fluor™ 488 Flow Cytometry Assay Kit, C10632, ThermoFisher Scientific). Intracellular staining for Ki67 and additional staining for PE conjugates was performed for 10 min at room temperature post EdU staining.

### Photoconversion of KikGR

Mice were anesthetized with a ketamine (100 mg per kg)-xylazine (10 mg per kg) mixture. The heads were shaved, skin midline incisions were made, and the skull was exposed. Photoconversion was performed using an Optogenetics-LED-Violet module (Prizmatix) for 2 min at an intensity of 5 with the light source placed 1 cm above the skull. Mice were then sutured and allowed to recover on a heating pad until responsive.

### Proteome Profiler Chemokine Array

Chemokines were semi-quantitatively determined from two-month-old WT mouse dural homogenates using the Proteome Profiler Mouse Chemokine Array Kit (R&D Systems) as per the manufacturer's instructions. Briefly, peeled cranial dura was peeled from the skull and placed into PBS with 1× Complete Mini ETA Protease inhibitor cocktail (Sigma Aldrich) and homogenized using a Mini Beadbeater (BioSpec Products) and 2.3-mm zirconia/silica beads (BioSpec Products). Triton X-100 was added to a final concentration of 1%, samples, samples were frozen at −80° C., thawed, and centrifuged at 1000 g for 10 minutes to pellet tissue. Three pooled dural homogenates were used for each sample, and samples were run in duplicate. Semi-quantitative analysis was performed via densitometric measurements using FIJI.

Spinal Cord scRNAseq Analysis for EAE and SCI Models

EAE scRNA-seq—Three pairs of female WT and UBC-GFP C57BL/6J mice were parabiotically joined at 2 months of age. Mice were allowed to recover for 60 days, where after experimental autoimmune encephalomyelitis (EAE) was induced in both pairs by MOG35-55 immunization. After 15 days, mice were sacrificed, and spinal cord tissue was collected from the WT parabionts. Tissues were dissociated, as described in “Single-cell isolations”, to generate a single-cell suspension and stained as described in “Flow cytometry and fluorescence-activated cell sorting”. Samples were sorted using a BD FACSAria II (BD Biosciences) based on the following phenotypes: Singlets, DAPI-CD45hiIVCD45−GFP− or Singlets, DAPI-CD45hiIVCD45−GFP+. Each sample represents pooled GFP− or GFP+ subsets from three pooled female mice at 4-5 months of age. Cells were sorted into 1.5-ml tubes with DMEM, pelleted, viability determined using trypan blue exclusion, and resuspended in 0.04% non-acetylated BSA. Samples were loaded onto a 10× Genomics Chromium platform for GEM and cDNA generation carrying cell- and transcript-specific barcodes and sequencing libraries constructed using the Chromium Single Cell 3′ Library & Gel Bead Kit v3. Libraries were sequenced on the Illumina NovaSeq6000, targeting a depth of 100,000 reads per cell.

Spinal cord injury scRNA-seq-Four pairs of female WT and UBC-GFP C57BL/6J mice were parabiotically joined at 2 months of age. Mice were allowed to recover for 60 days, where spinal cord injury was performed in both pairs by a crush at Th9 level with fine forceps. After 3 days, mice were sacrificed, and spinal cord tissue was collected from the WT parabionts. Tissues were dissociated, as described in “Single-cell isolations”, to generate a single-cell suspension and stained as described in “Flow cytometry and fluorescence-activated cell sorting”. Samples were sorted using a BD FACSAria II (BD Biosciences) based on the following phenotypes: Singlets, DAPI-CD45hiIVCD45−GFP− or Singlets, DAPI-CD45hiIVCD45−GFP+. Each sample represents pooled GFP− or GFP+ subsets from four pooled female mice at 4-5 months of age. Cells were sorted into 1.5-ml tubes with DMEM, pelleted, viability determined using trypan blue exclusion, and resuspended in 0.04% non-acetylated BSA. Samples were loaded onto a 10× Genomics Chromium platform for GEM and cDNA generation carrying cell- and transcript-specific barcodes and sequencing libraries constructed using the Chromium Single Cell 3′ Library & Gel Bead Kit v3. Libraries were sequenced on the Illumina NovaSeq6000, targeting a depth of 100,000 reads per cell.

Data Preprocessing.: Reads were aligned to the mm10 transcriptome using the Cellranger software pipeline (version 4.0) provided by 10× genomics. The resulting filtered gene by cell matrices of UMI counts for each sample were read into R using the read10×Counts function from the Droplet Utils package. Filtering was applied in order to remove low-quality cells by excluding cells expressing fewer than 200 or greater than 6000 unique genes, having fewer than 2000 or greater than 50000 UMI counts, as well as cells with greater than 25% mitochondrial gene expression. Expression values for the remaining cells were then merged by gene symbol into one data frame and normalized using the scran and scater packages. The resulting log 2 values were transformed to the natural log scale for compatibility with the Seurat (v3) pipeline.

Dimensionality Reduction and Clustering.: The filtered and normalized matrix was used as input to the Seurat pipeline and cells were scaled across each gene before the selection of the top 2000 most highly variable genes using variance stabilizing transformation. Principal Components Analysis was conducted and an elbow plot was used to select the first six principle components for tSNE analysis and clustering. Shared nearest neighbor (SNN) clustering optimized with the Louvain algorithm, as implemented by the Seurat FindClusters function was performed before manual annotation of clusters based on the expression of canonical gene markers. Clusters were then collapsed based on common cell types to result in seven clusters.

Differential Expression.: For analysis of differentially expressed genes between conditions, each cluster was filtered to include genes that had at least four transcripts in at least four cells, then the top 2000 highly variable genes were determined and included for further analysis using the Single Cell Experiment model GeneVar and get Top HVGs functions. After filtering, observational weights for each gene were calculated using the ZINB-WaVE zinbFit and zinbwave functions. These were then included in the edgeR model, which was created with the glmFit function, by using the glmWeightedF function. Results were then filtered using a Benjamini-Hochberg adjusted p-value threshold of less than 0.05 as statistically significant. As part of that function, an F test is applied to the generalized linear regression model with adjusted denominator degrees of freedom to account for the downweighting in the zero-inflation model.

Pathway Enrichment: Over-representation enrichment analysis with Fisher's Exact test was used to determine significantly enriched Gene Ontology (GO) terms (adj. P<0.05) for the sets of significantly differentially expressed genes between young and old in each tissue as a whole and at the cluster level. For each gene set, genes were separated into up- and downregulated, and separately the enrichGO function from the clusterProfiler package was used with a gene set size set between 10 and 500 genes and p-values adjusted using the Benjamini-Hochberg correction.

Whole-Dura scRNAseq Analysis

Whole-dura scRNA-seq analysis was performed on samples previously described. Briefly, single-cell suspensions were generated from five individual young and old dura per experiment, two experiments, and 10 dura samples per age total. Samples were stained with DAPI and sorted using a BD FACSAria II (BD Biosciences). Cells were sorted as DAPI-singlets, into 1.5-mL tubes with DMEM, pelleted, viability determined using trypan blue exclusion, and resuspended in 0.04% non-acetylated BSA. The sorted young and old dural samples were loaded onto a 10× Genomics Chromium platform for GEM and cDNA generation carrying cell- and transcript-specific barcodes and sequencing libraries constructed using the Chromium Single Cell 3′ Library & Gel Bead Kit v3. Libraries were sequenced on the Illumina NovaSeq6000, targeting a depth of 100,000 reads per cell.

Data Preprocessing.—Reads were aligned to the mm10 transcriptome using the Cellranger software pipeline (version 4.0) provided by 10× genomics. The resulting filtered gene by cell matrices of UMI counts for each sample were read into R using the read10×Counts function from the Droplet Utils package. Filtering was applied in order to remove low-quality cells by excluding cells expressing fewer than 200 or greater than 600 unique genes, having fewer than 1500 or greater than 50000 UMI counts, as well as cells with greater than 25% mitochondrial gene expression. Expression values for the remaining cells were then merged by gene symbol into one dataframe and normalized using the scran and scater packages. The resulting log 2 values were transformed to the natural log scale for compatibility with the Seurat (v3) pipeline.

Dimensionality Reduction and Clustering—The filtered and normalized matrix was used as input to the Seurat pipeline and cells were scaled across each gene before the selection of the top 2000 most highly variable genes using variance stabilizing transformation. Principal Components Analysis was conducted and an elbow plot was used to select the first ten principle components for tSNE analysis and clustering. SNN clustering optimized with the Louvain algorithm, as implemented by the Seurat FindClusters function was performed before manual annotation of clusters based on the expression of canonical gene markers. Clusters were then collapsed based on common cell types to result in 17 clusters.

Cell-Cell Interaction Analysis—To evaluate potential cell-cell ligand-receptor interactions in an unbiased manner, the RNAMagnet package was utilized with RNAMagnetSignaling and the top signaling pair molecules were examined for both monocytes and neutrophils with each other cell type present in the dataset.

### Experimental Study Design

All experiments were blinded, where possible, for at least one of the independent experiments. For irradiation and bone-marrow transfer experiments and skull transplantation studies, blinding was not possible due to obvious differences in the physical appearance of mice. No data were excluded, with the exception of skull transplantation studies, where mice were excluded if they displayed no GFP signal in the skull bone marrow after 7 or 30 days, demonstrating that the surgery was not successful. Animals from different cages were randomly assigned to different experimental groups. Statistical methods were not used to re-calculate or predetermine study sizes but were based on similar experiments previously published. Statistical tests for each experiment are provided in figure legends. All data represent biological replicates. Statistical analysis was performed using Prism 8.0 (GraphPad Software Inc.).

In an attempt to understand the origin of border myeloid cells under homeostasis, we parabiotically joined the circulations of UBC-GFP and wild-type (WT) mice, allowing us to visualize host and fluorescent donor-derived cells, and performed flow cytometric analyses of the blood, brain-associated tissues (cranial dura mater and spinal dura mater), hematopoietic organs (skull bone marrow, vertebrae bone marrow, and femur bone marrow), and peripheral control tissues (spleen and liver) after 60 days of pairing (FIGS. 1A, 1B, and 1C). To ensure our analysis only examined true parenchymal populations and not blood contaminants, mice received intravenous anti-CD45 antibodies 2 min prior to euthanasia, labeling blood and vascular-associated leukocytes that were subsequently excluded in our gating strategy (FIG. 1B). Analysis of WT parabionts demonstrated that although blood chimerism for total CD45+ cells reached a 50:50 ratio (FIG. 14A), neither neutrophils nor monocytes ever reached perfect chimerism in the blood. After two months, a GFP:WT cell ratio of ˜30:70 was achieved (FIGS. 1D and 1E). The reduced chimerism of these myeloid cells was likely due to the short half-life of these cells as well as active myelopoiesis occurring in the bone marrow.

Despite imperfect blood chimerism of Ly6C+ monocytes and neutrophils, GFP+ cell proportions detected in the cranial dura and spinal dura were significantly lower than that of blood (FIGS. 1D and 1E). By comparison, the myeloid chimerisms displayed in other peripheral tissues were more akin to the proportions present in the blood (FIGS. 1D and 1E). These data suggested there was a substantial pool of monocytes and neutrophils located along the brain borders that had not originated from the UBC-GFP parabiont. Cranial and spinal dural CD4 T cells from the UBC-GFP parabiont were not significantly different from the blood chimerism, suggesting a blood origin as described previously (FIG. 1F). Furthermore, GFP+Ly6c− monocytes, and B cells displayed different levels of chimerism in distinct tissues, specifically GFP+ B cells appeared to be less represented at the brain borders, also suggesting a non-blood origin (FIGS. 14B and 14C). Additional components of the dural myeloid compartment including macrophages, conventional dendritic cells (cDC)1s, and cDC2s also displayed a low proportion of GFP+ cells compared to the non-border associated spleen pool (FIGS. 14D, 14E, and 14F). Analysis of several bone marrow niches confirmed that the majority of detected hematopoietic progenitors Lin-/SCA-1/C-Kit (LSK) were GFP− cells, as expected from an active WT proliferative niche (FIG. 14G). We corroborated these flow cytometry data by immunohistochemistry (IHC) and demonstrated only a minor proportion of GFP+CCR2+ and GFP+Ly-6B.2+ cells in WT host tissues (FIG. 1G). Utilizing CD45.1 and CD45.2 pairs as parabionts, we observed a similar imbalance towards host-derived monocytes and neutrophils within the dura compared to the blood chimerism, suggesting the observed findings are consistent across different parabiotic pairs (FIGS. 14H and 141). Thus, a substantial pool of non-blood-derived monocytes and neutrophils appear to populate the cranial and spinal dura.

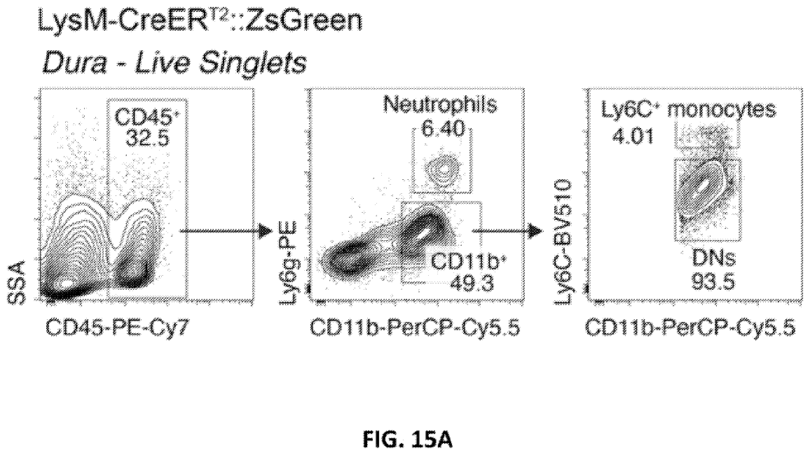

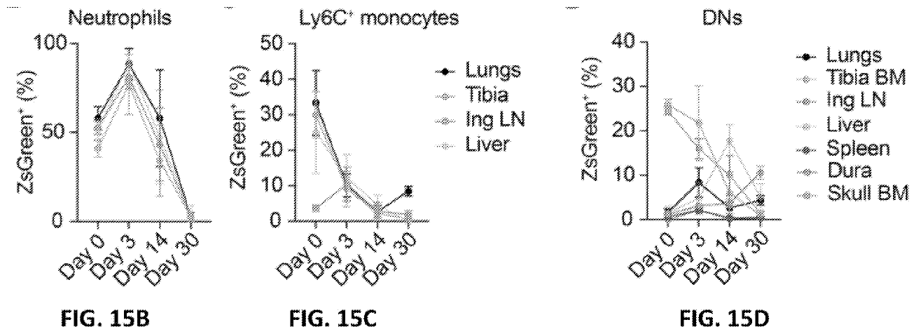

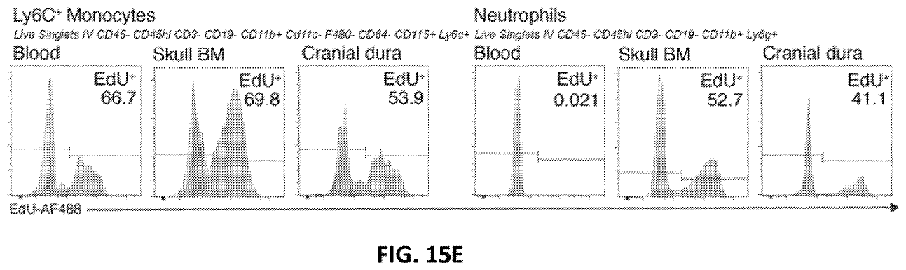

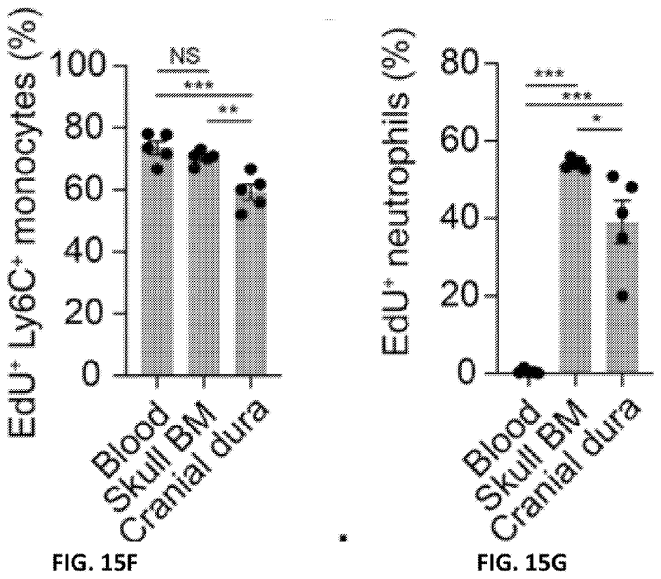

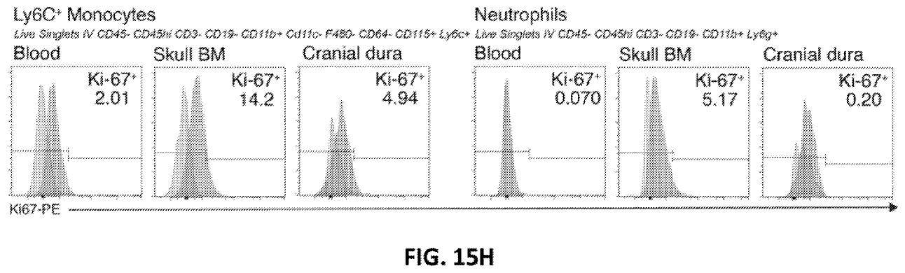

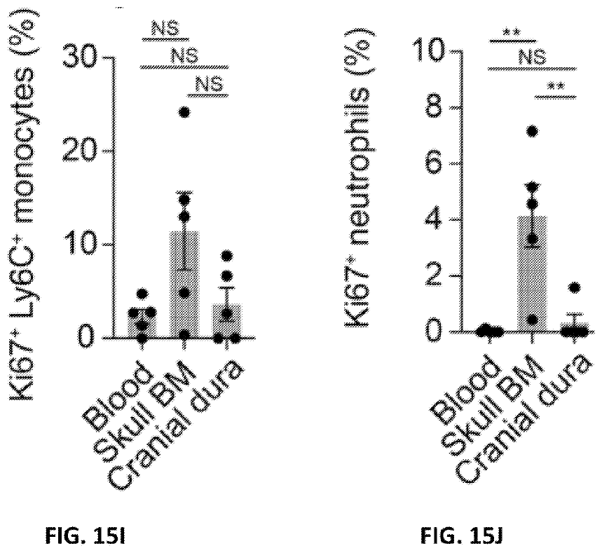

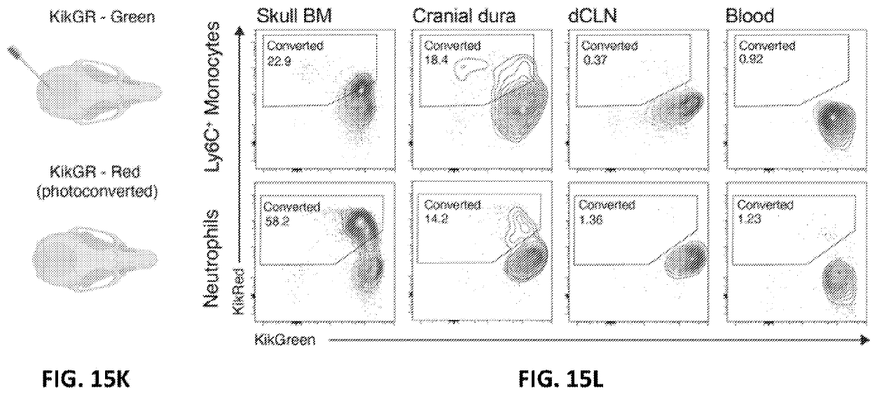

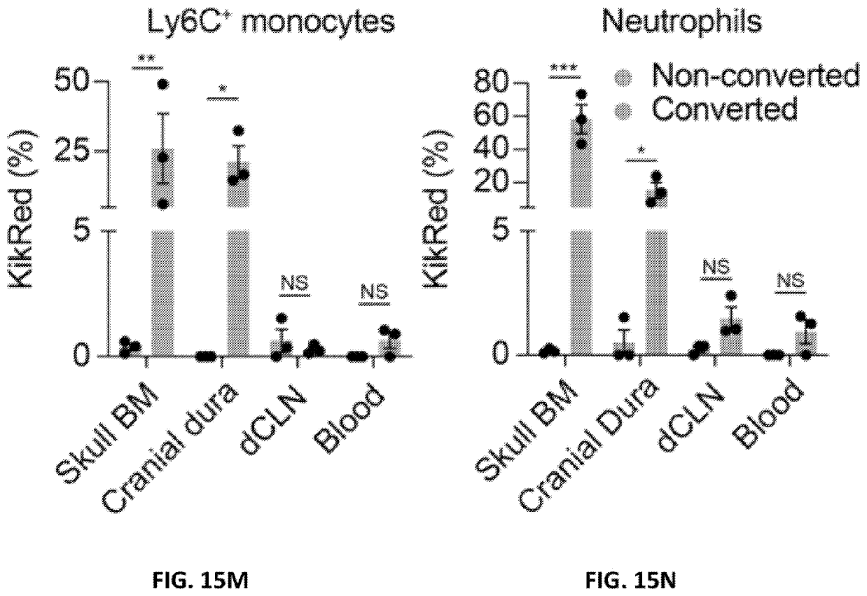

Next, we investigated whether the dura harbored monocyte progenitor cells that could maintain a local monocyte pool. Using flow cytometry, we could efficiently identify monocyte-dendritic cell progenitors (MDPs) and monocyte-committed progenitors (cMoPs) in the skull bone marrow, yet these were entirely absent in the cranial dura (FIGS. 14J, 14K, 14L, 14M, 14N, 14O, 14P, 14Q, 14R, and 14S). To rule out the possibility that the low levels of meningeal chimerism observed for Ly6C+ monocytes and neutrophils could be attributed to the proliferation and self-renewal of those cells in the tissue, we explored the turnover of these myeloid cells using a tamoxifen-inducible fate-mapping Cre recombinase system driven by the pan-myeloid promoter LysM to express the fluorescent protein ZsGreen (FIG. 1H). Following tamoxifen injections over 3 consecutive days, blood monocytes and neutrophils reached a recombination efficiency of 36% and 87%, respectively, and after 30 days almost no circulating ZsGreen+ cells could be detected (FIGS. 1I, 1J, and 1K). Notably, the fate-mapping efficiency of the blood was mirrored by that in the dura across all investigated time points, indicating that the half-lives of the myelomonocytic cells in the blood and in the meningeal spaces were comparable (FIGS. 1H, 1I, 1J, 1K, 15A, 15B, 15C, and 15D). To rule out the possibility that the unlabeled fraction of monocytes and neutrophils provides a local pool of self-sustaining tissue-resident cells we performed in vivo EdU labeling and Ki-67 staining. Analysis of blood, cranial dura, and skull bone marrow confirmed that 24 hours after two EdU pulses, the majority of Ly6C+ monocytes and neutrophils in the dura incorporated EdU, yet they displayed low levels of Ki-67 (FIGS. 15E, 15F, 15G, 15H, 15I, and 15J). Thus, these cells appeared to originate from a proliferating source but likely experienced fast turnover in these tissues and low proliferation capabilities outside the bone marrow niche. Moreover, dural neutrophils displayed the same level of EdU incorporation as the skull bone marrow pool, whereas blood neutrophils were almost entirely devoid of EdU+ cells, suggesting dural neutrophils may arise from the nearby bone marrow sources (FIGS. 15E, 15F, 15G, 15H, 15I, and 15J). Finally, we investigated if the dural Ly6C+ monocyte and neutrophil pool used meningeal lymphatics to egress from the tissues. For this, we used KikGR transgenic mice with KikGreen to KikRed photoconvertible fluorescent protein. Although neutrophils and Ly6C+ monocytes were present in the draining deep cervical lymph nodes, 24 hours after the final photoconversion, no significant difference in KikRed cells could be detected in the deep cervical lymph nodes, compared to unconverted controls (FIGS. 15K, 15L, 15M, and 15N). Thus, replenishment of dural meningeal Ly6C+ monocytes and neutrophils appears to come from a local source and is not due to homeostatic maintenance within the tissue itself or from blood.

Skull Bone Marrow Supplies Brain Borders with Myeloid Cells

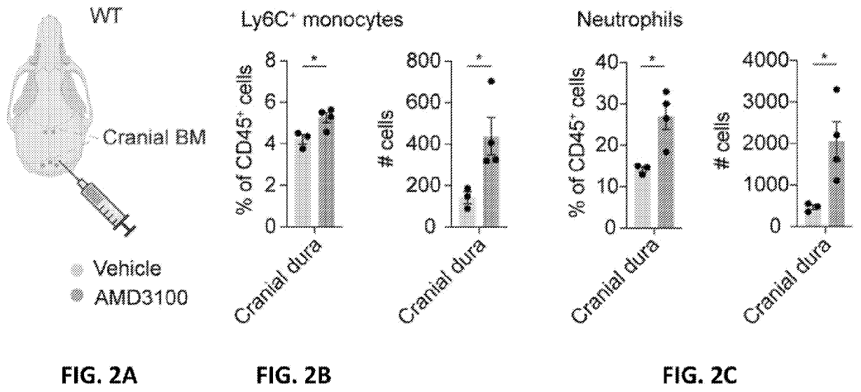

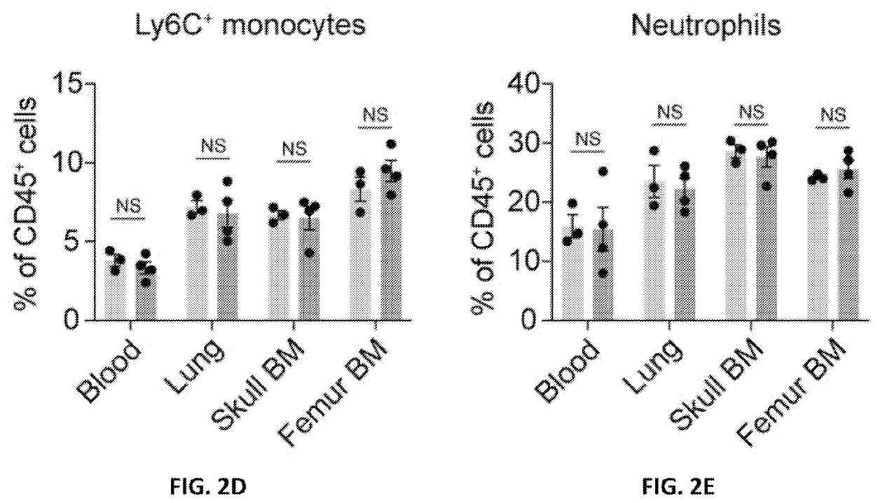

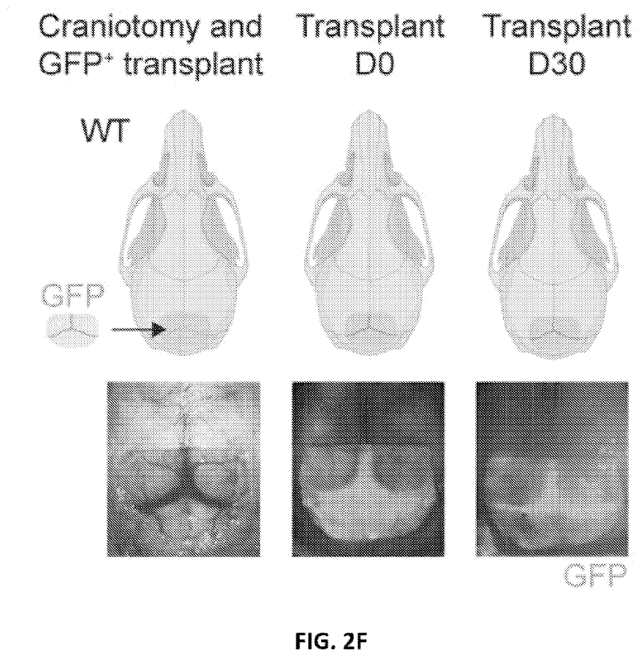

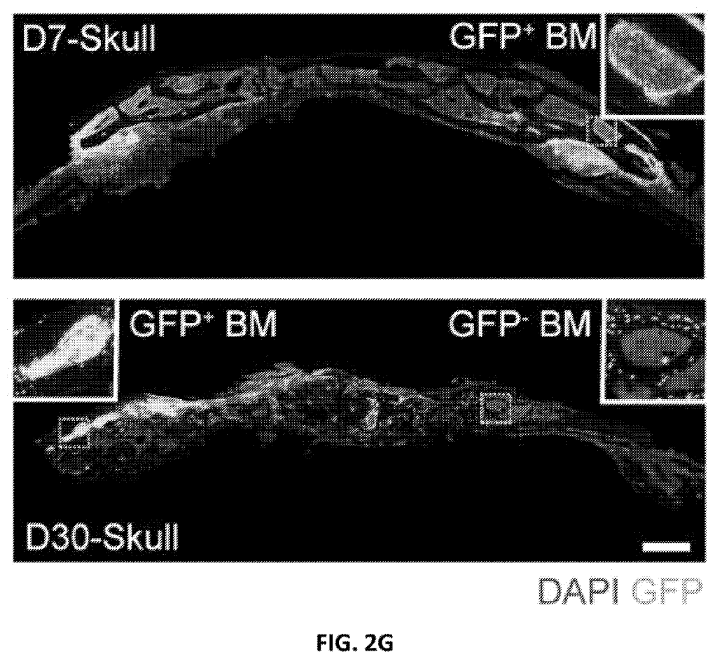

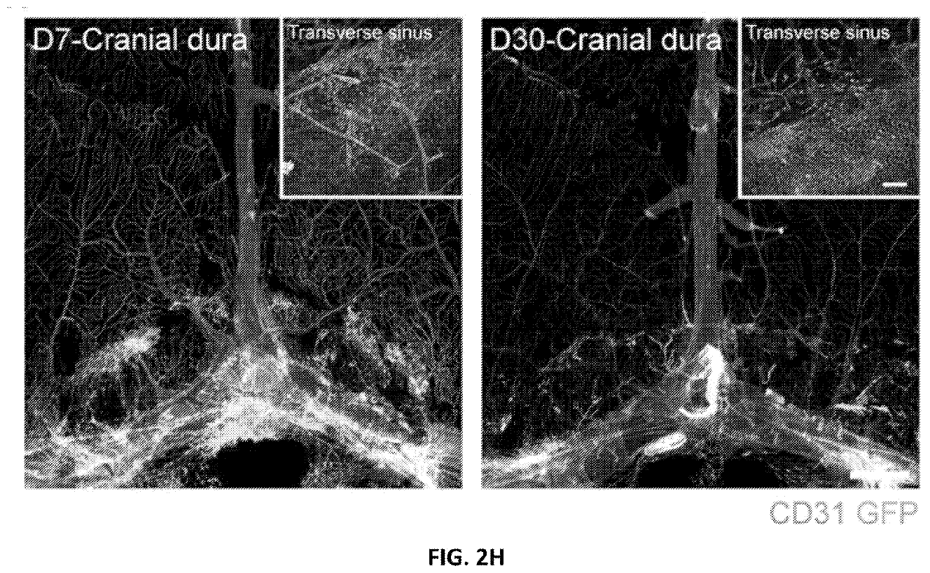

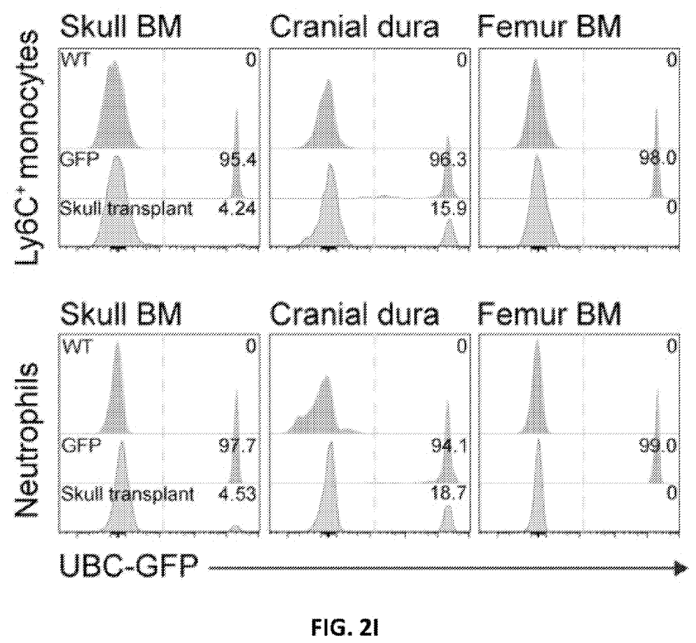

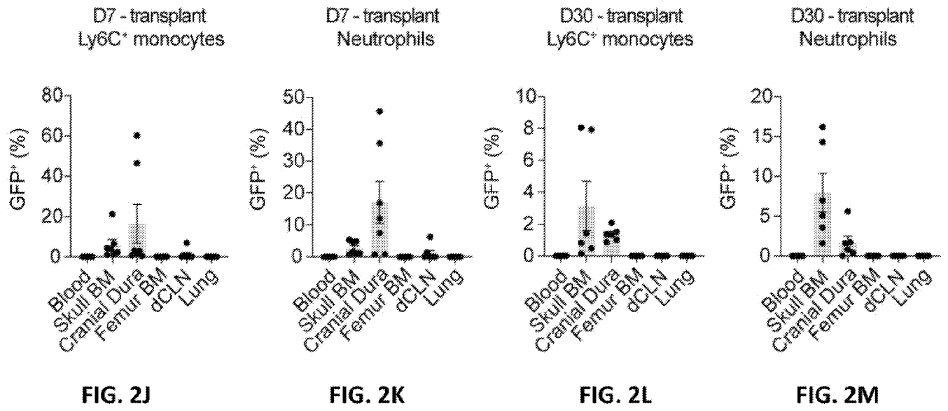

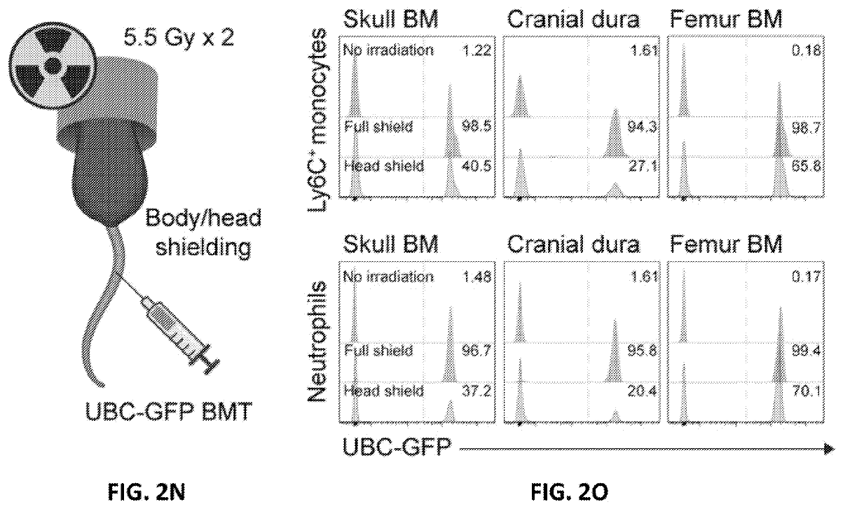

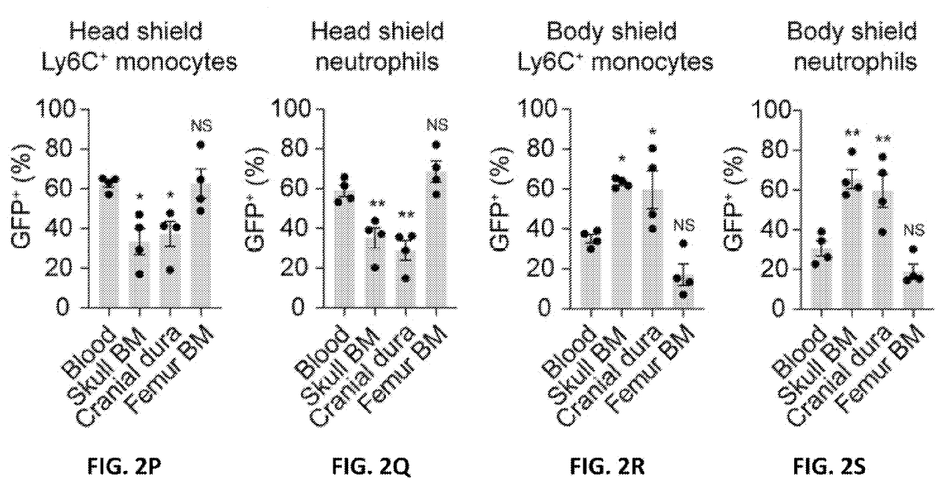

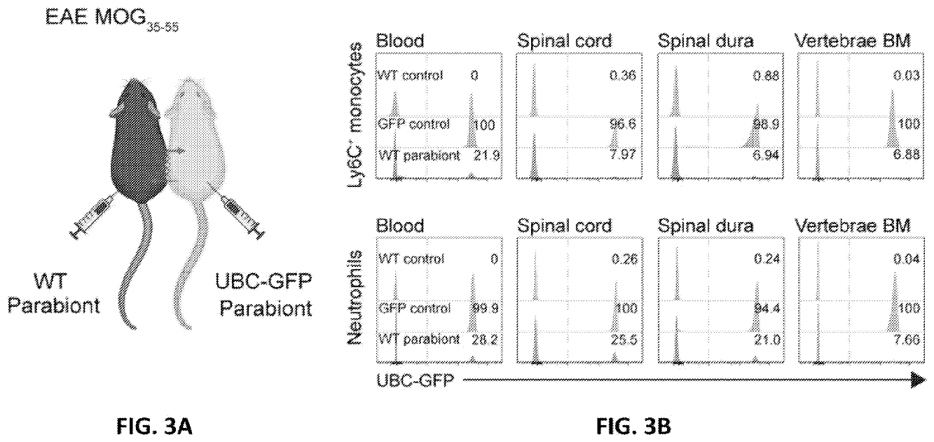

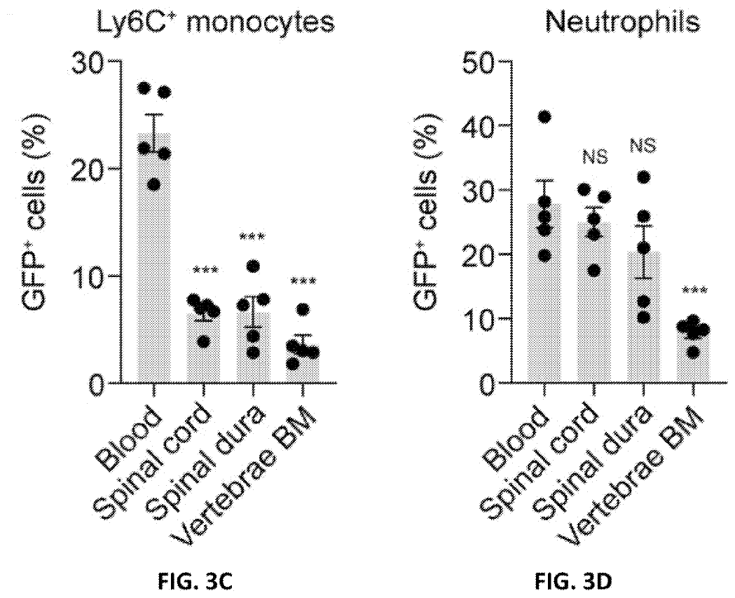

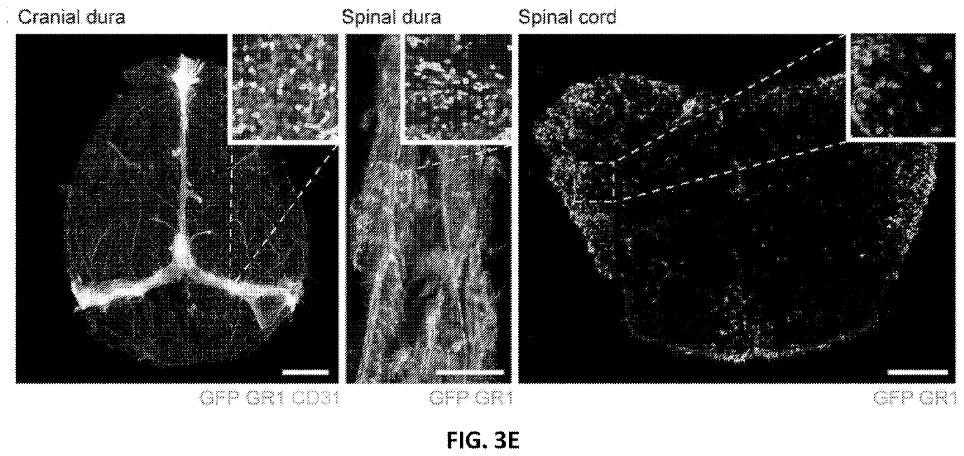

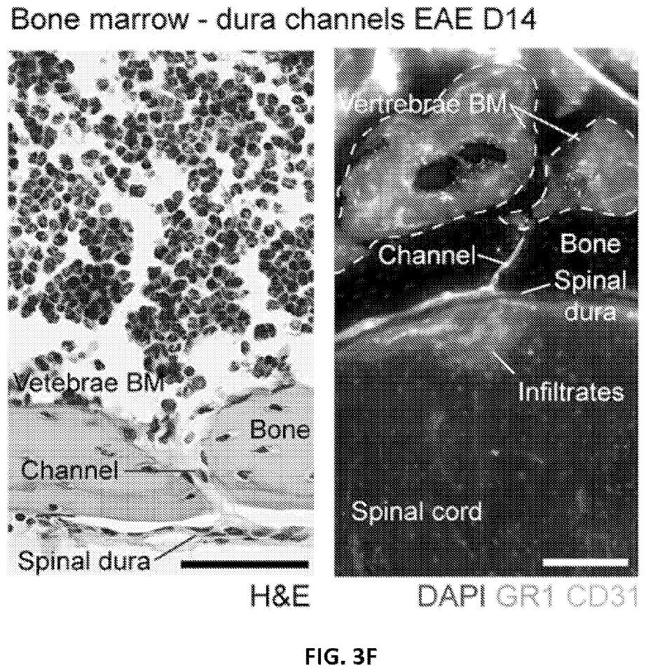

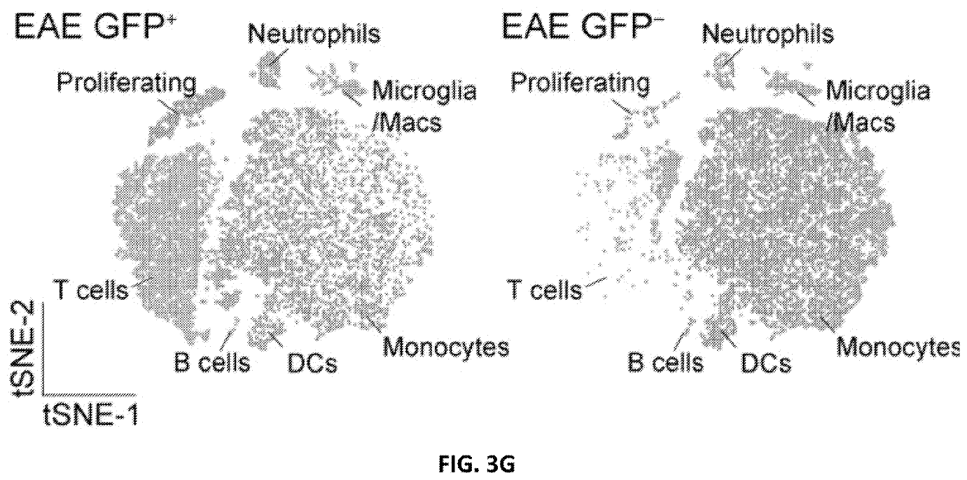

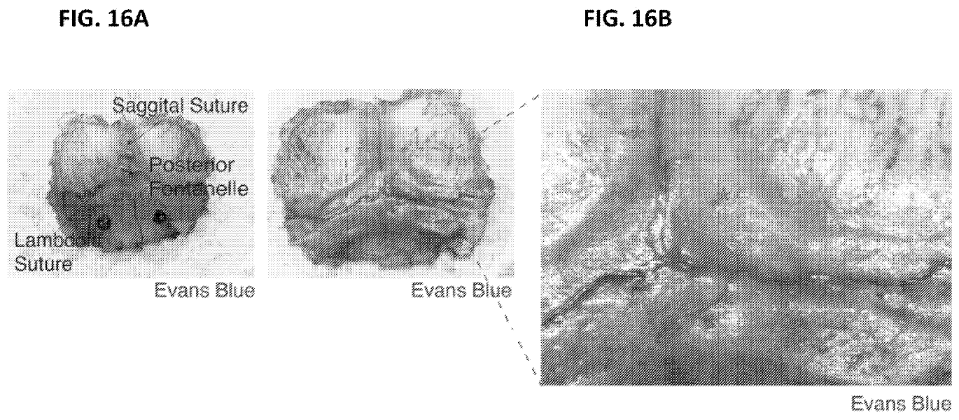

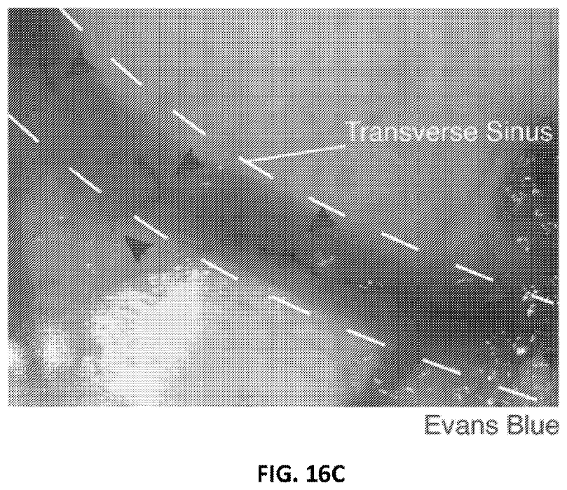

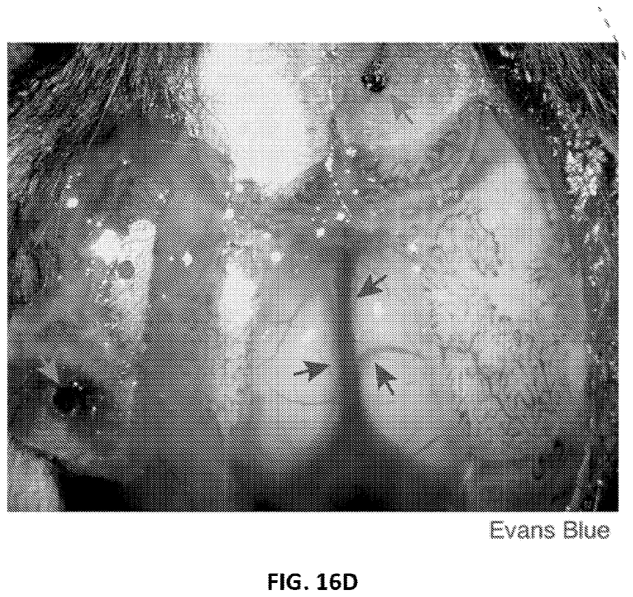

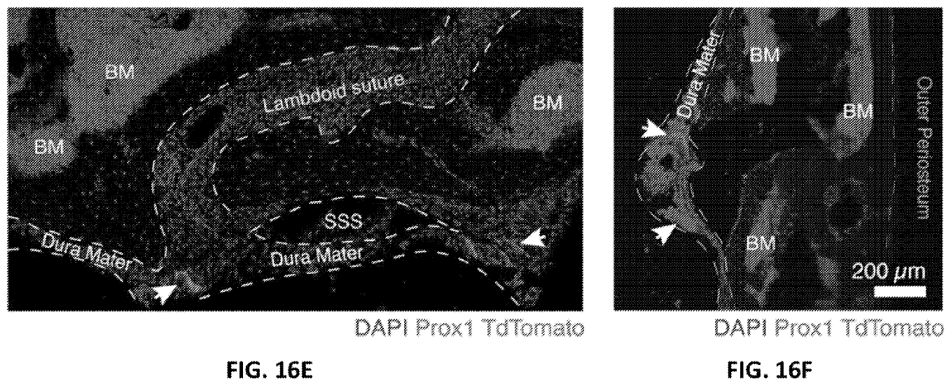

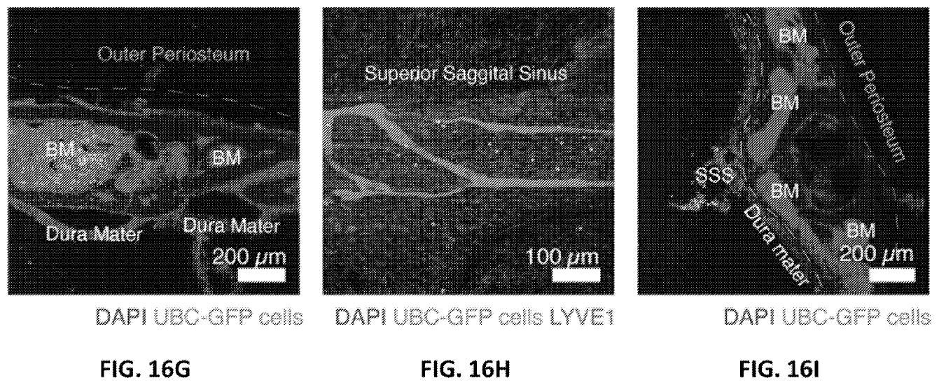

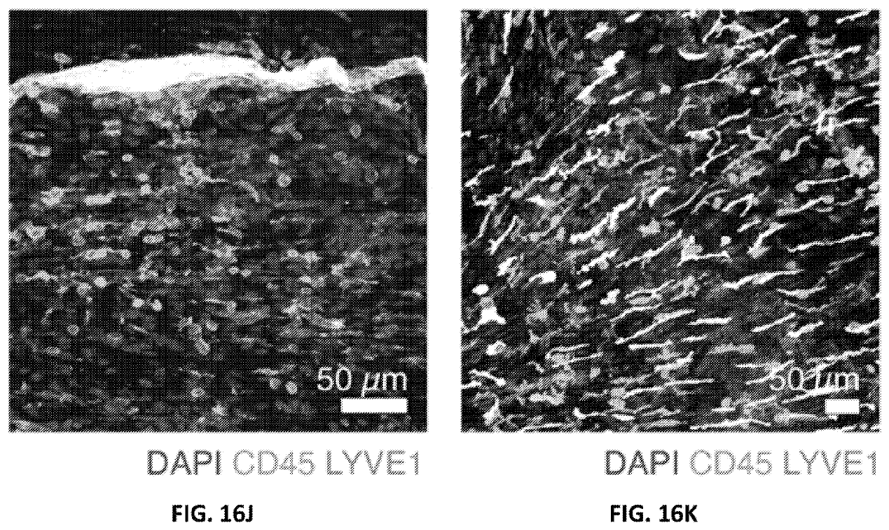

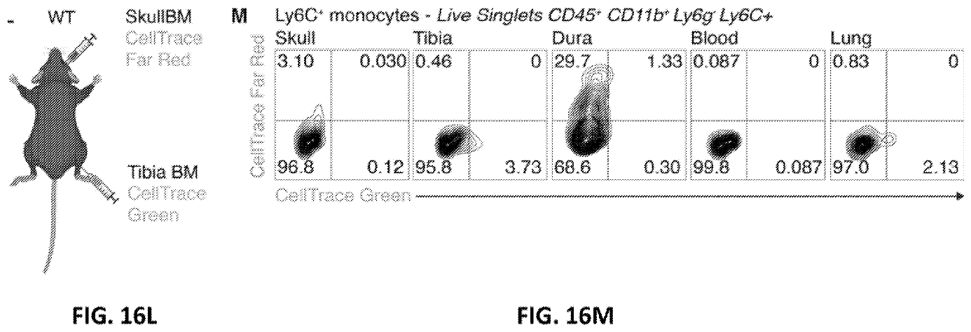

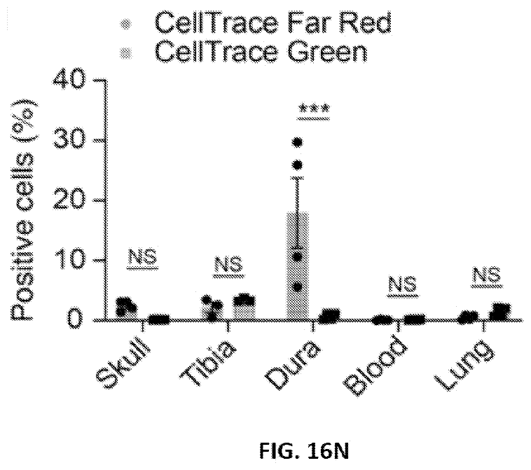

Direct connections between the skull and the dura through ossified channels harboring blood vessels extend from the bone marrow to the CNS and allow neutrophils and tumor cell migration during inflammatory or pathological states. One prior strategy for neutrophil tracking utilized injections into skull bone marrow to label brain-infiltrating cells. However, on reproducing this approach in an attempt to assess a bone marrow origin for homeostatic dural myeloid cells, we found it unsuitable for this purpose as it resulted in immediate leakage of the tracers into the dura mater and their subsequent drainage by meningeal lymphatic vessels (FIGS. 16A, 16B, 16C, 16D, 16E, 16F, 16G, 16H, 16I, 16J, 16K, 16L, 16M, 16N). Despite caveats of these direct bone marrow injections, we found that a brief topical application of AMD3100, a CXCR4 antagonist, to cranial bone marrow exposed by skull thinning, promoted myeloid cell egress. This resulted in significant elevations in Ly6C+ monocytes and neutrophils in the underlying dura mater, without altering proportions in the blood, lung, or other bone marrow niches, suggesting direct migration from local skull bone marrow (FIGS. 2A, 2B, 2C, 2D, and 2E). We did not observe a change in the number of myeloid cells in the skull bone marrow 24 hours following AMD3100 administration. This is likely due to the number of monocytes and neutrophils infiltrating the dura representing only a very small proportion of the CNS-associated bone marrow niche.

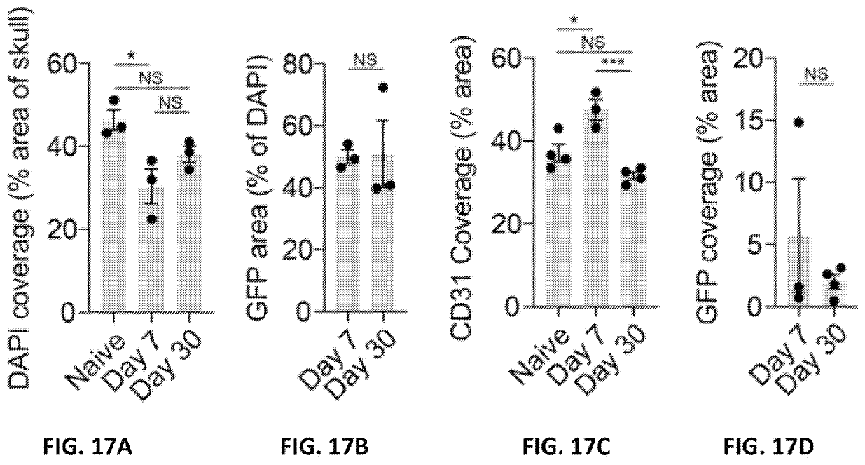

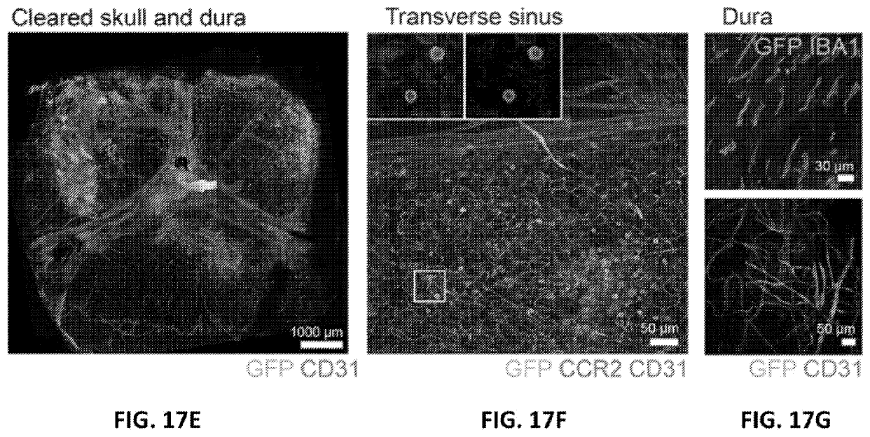

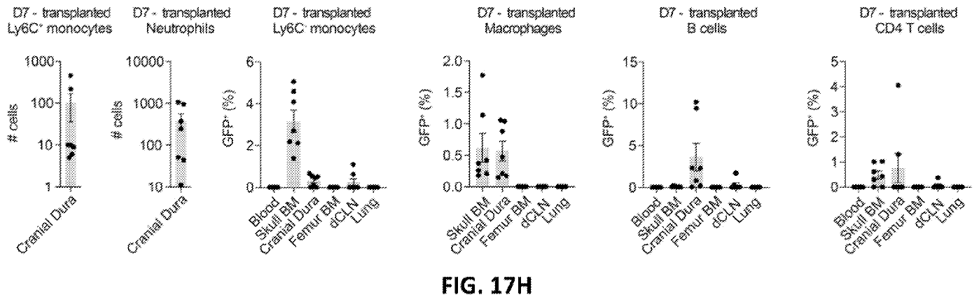

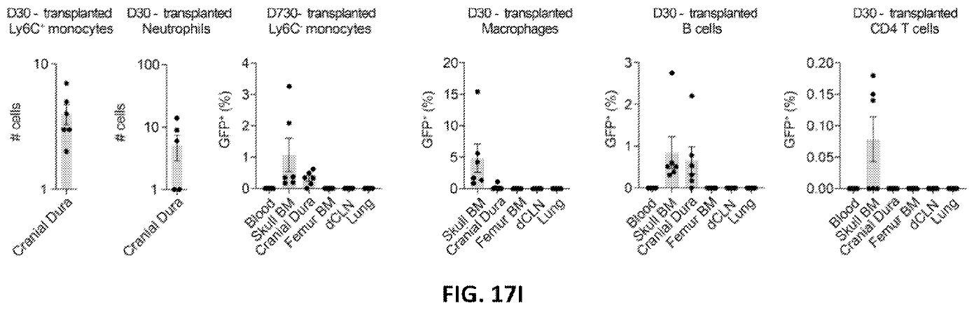

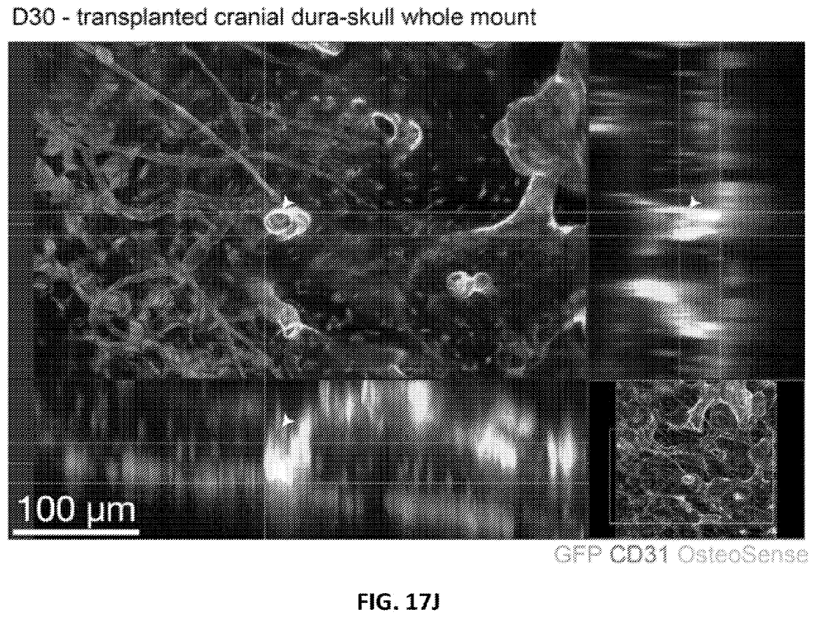

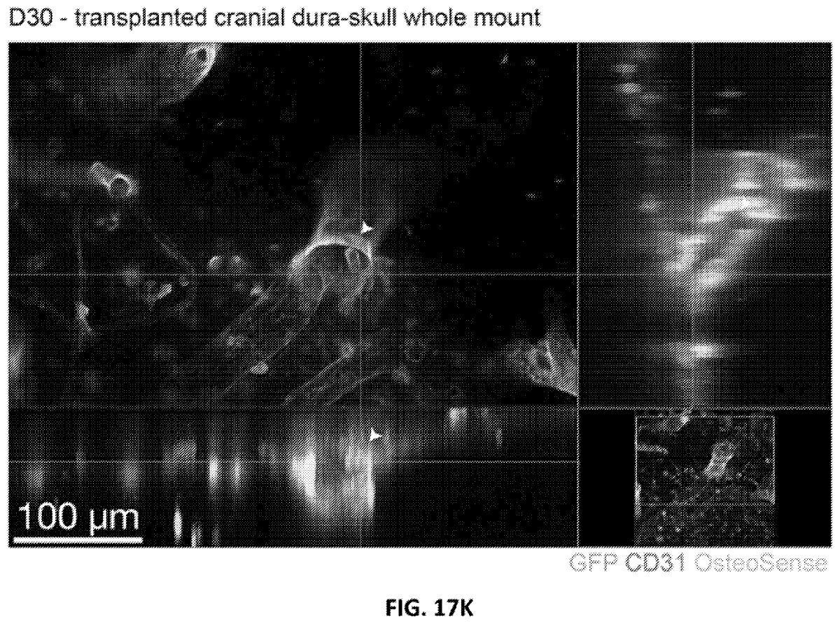

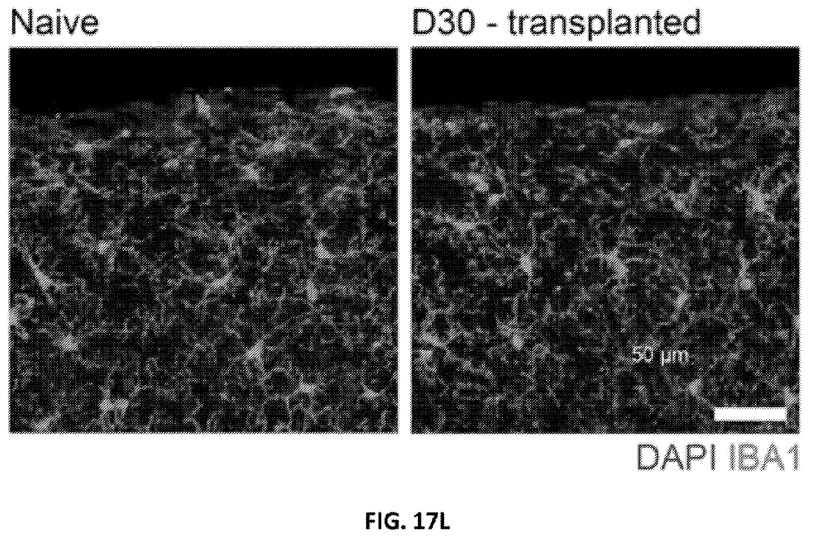

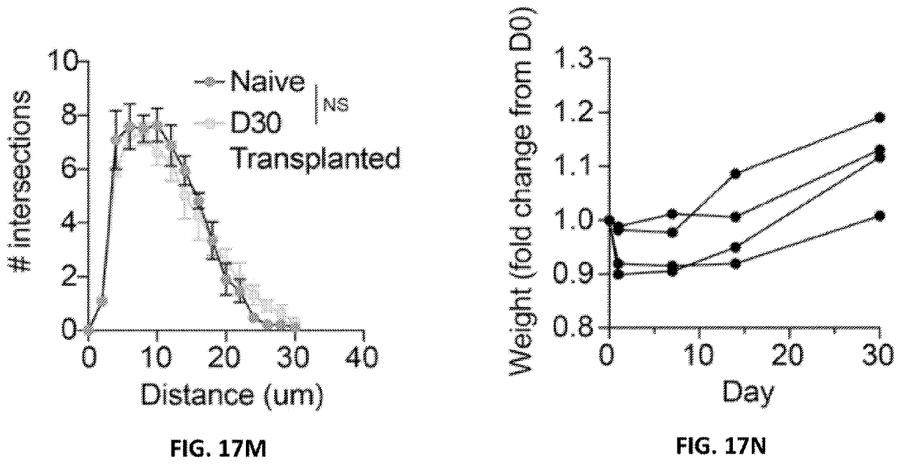

These data prompted us to look closely into the possibility that the skull bone marrow is a source for meningeal myeloid cells. Therefore, we developed a calvaria bone-flap transplantation, in which a piece of the calvarium (˜6×4 mm) containing a substantial bone marrow reservoir was transplanted from UBC-GFP mice to cover a skull window of similar size created in WT mice whose dura mater was carefully left intact (FIG. 2F). IHC analysis of transplanted calvaria flaps 7 and 30 days after transplantation revealed the continued presence of GFP+ bone marrow, though some sites were partially repopulated by non-GFP expressing cells (FIGS. 2G, 17A and 17B). Consistent with sterile injury-induced dural angiogenesis, vascular remodeling was observed at 7 days but returned to the coverage of naive mice after 30 days (FIGS. 2H, 17C, and 17D). The transplanted flap contained cranial sutures necessary for skull repair, and after 1 month, transplanted bone was incorporated into the WT skull (FIG. 17E) and we detected GFP+ cells including CCR2+ monocytes, IBA1+ macrophages, and CD31+ vasculature, in the underlying dura of the transplanted calvaria flap (FIGS. 17F and 17G). Flow cytometric analysis of blood, cranial dura, and skull bone marrow confirmed that although the donor pool of GFP+ cells in the transplanted skull bone marrow niche was low, it was able to give rise to dural Ly6C+ monocytes and neutrophils even 30 days posts transplant (FIGS. 2I, 2J, 2K, 2L, 2M, 17H, and 17I). Demonstrating a direct route for dural myeloid infiltration from the transplanted skull, we observed previously described vascular channels connecting the transplanted skull to the dura mater (FIGS. 17J, 17K, and 16). Analysis of the underlying cortex revealed no microglial activation via Sholl analysis after 30 days (FIGS. 17L and 17M) and following an initial weight loss after transplantation, mice resumed a normal weight gain, suggesting minimal chronic perturbations using this model (FIG. 17N).

We next used an irradiation regime to further exclude the possibility that the observed phenotype following parabiosis or calvaria bone flap transplantation strategies is inflammation driven (FIG. 2N). Mice underwent lethal irradiation coupled with different shielding strategies and were reconstituted with UBC-GFP-derived bone marrow. As expected, cranial dural Ly6C+ monocytes and neutrophils in head-shielded mice, whose bodies were irradiated, showed similar levels of chimerism as skull bone marrow. Chimerism at both sites was significantly lower than in the blood and femur bone marrow (FIGS. 2O, 2P, and 2Q). The body-shielding strategy, in which the head was irradiated, displayed the opposite effect with significantly higher proportions of GFP+ cells in the skull bone marrow and cranial dura, compared to blood or femur bone marrow (FIGS. 2R and 2S).

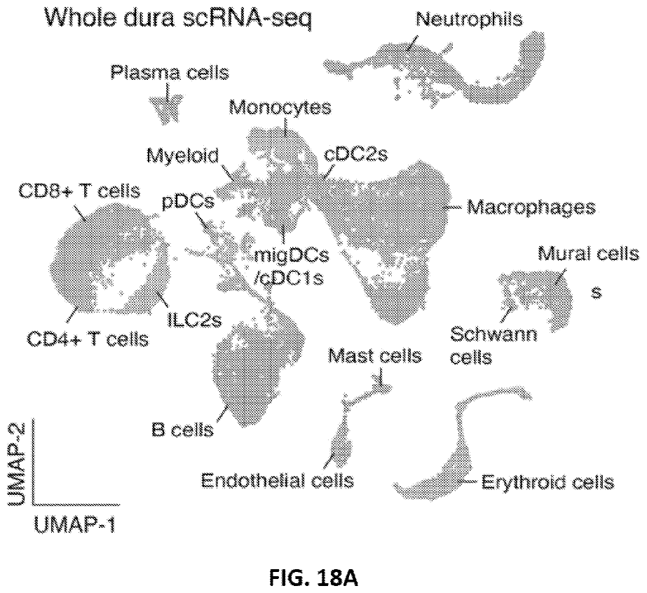

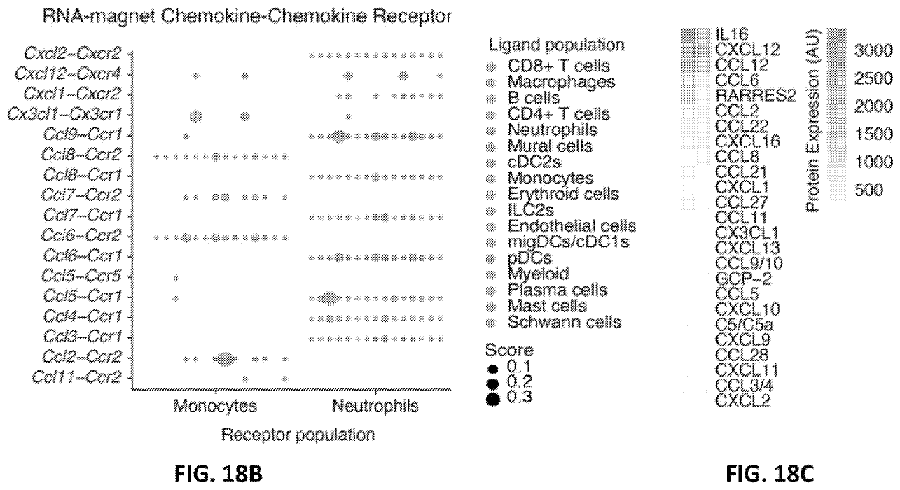

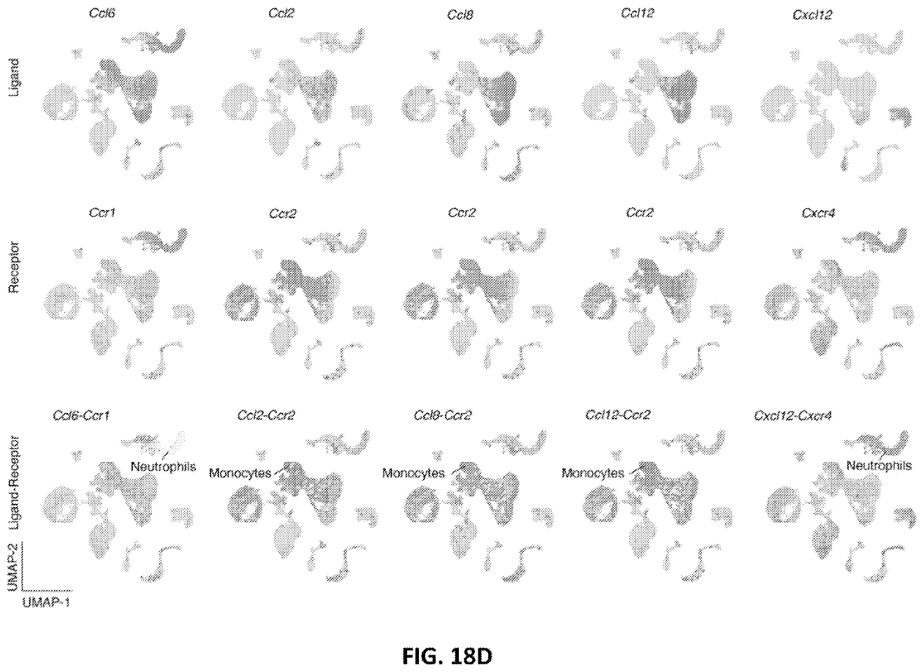

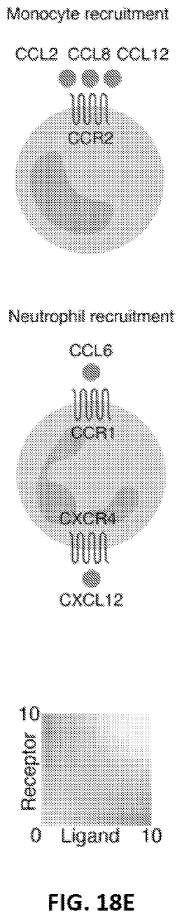

To explore dural-derived factors that could recruit bone marrow-derived myeloid cells, we utilized RNA-magnet algorithms from whole dura single-cell RNA sequencing (scRNA-seq) analysis, to unbiasedly identify ligand expression for monocyte and neutrophil chemokine receptors (FIGS. 18A and 18B). We then performed semi-quantitative chemokine protein expression analysis from whole dura homogenates, to confirm the true presence of predicted ligands (FIG. 18C). We identified high expression of CCL2, CCL12, and CCL8 which can recruit monocytes through CCR2 signaling, and CCL6 which can recruit neutrophils via CCR1. This suggested that the homeostatic dura contains appropriate signals for local myeloid recruitment from adjacent bone marrow (FIGS. 18C, 18D, and 18E). Thus, under homeostatic conditions, the myeloid niche distributed along the brain borders receives substantial input from the skull bone marrow, which appears to act as a critical dispenser of myeloid cells. This provides an example of a healthy tissue hosting myeloid cells that are continuously replenished by a source that does not use blood as a major route.

### The Inflamed CNS is Infiltrated by Blood and CNS-Adjacent Bone Marrow-Derived Myeloid Cells

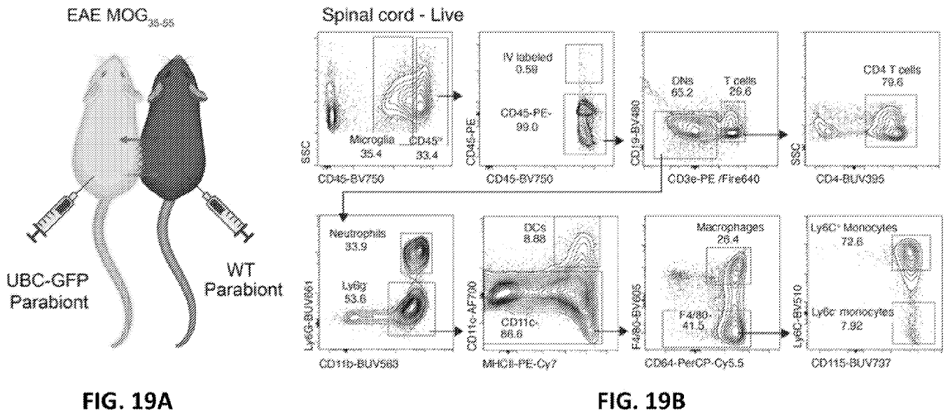

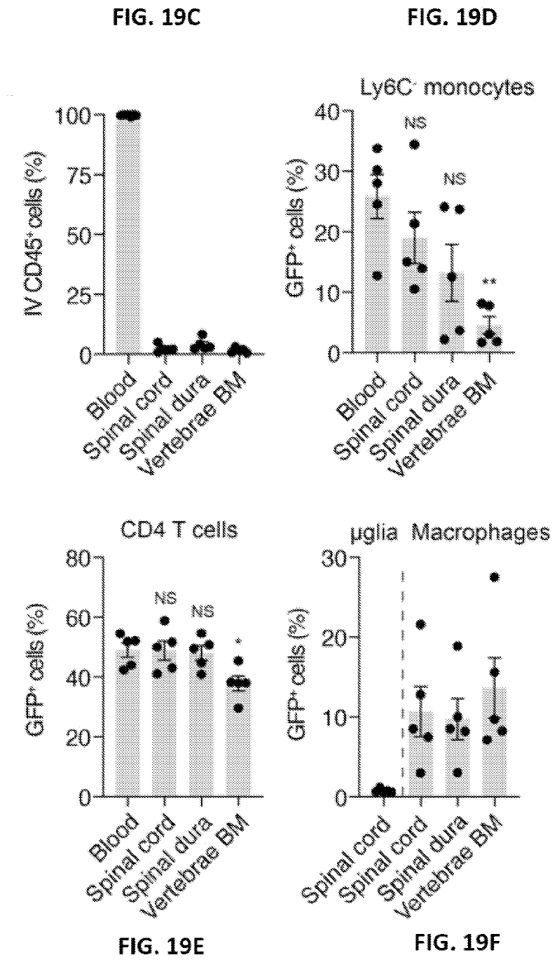

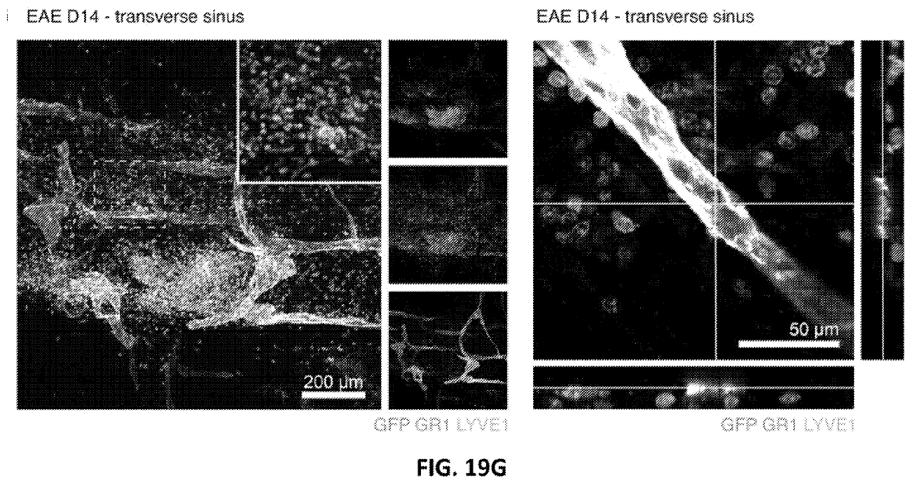

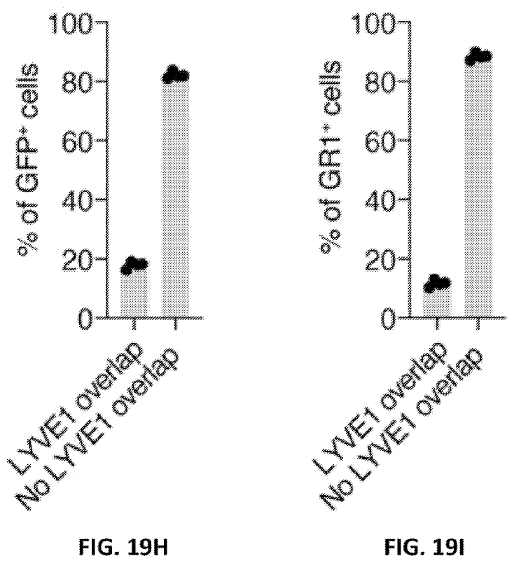

Intrigued by the large proportion of non-blood-derived monocytes and neutrophils residing in the dura, we speculated that these cells may be positioned for a prompt response in the event of parenchymal CNS damage. We proceeded to investigate this scenario using parabiotic pairing in three CNS-injury models, experimental autoimmune encephalomyelitis (EAE), spinal cord injury, and optic nerve crush injury. For EAE experiments, WT and UBC-GFP mice were joined parabiotically for 60 days and both groups were immunized with myelin oligodendrocyte glycoprotein (MOG) peptide in complete Freud's adjuvant (CFA) to induce EAE. The lumbar spinal cord, lumbar vertebral bone marrow, spinal dura, and blood were collected from the WT parabionts 15 days after EAE induction and stained for flow cytometry and IHC (FIGS. 3A, 19A, and 19B). In agreement with their homeostatic presence, a substantial pool of dural and CNS-infiltrating Ly6c+ monocytes did not originate from the blood, indicated by their significantly lower GFP+ proportion compared to the blood chimerism (FIGS. 3B and 3C). Interestingly, the same was not observed for neutrophils, CD4 T cells, and Ly6C− monocytes, suggesting blood was the major source of these infiltrates at this time point (FIGS. 3D, 19C, 19D, and 19E). Dural and spinal cord GFP+ macrophages were also observed, suggesting infiltrating monocytes can differentiate under these inflammatory conditions, though not at a sufficient amount to explain the chimerism discrepancy (FIG. 19F). In agreement with flow cytometry analysis, IHC for cranial dura, spinal dura, and spinal cord, revealed that infiltrating GR1+ cells within WT tissues were rarely GFP+(FIG. 3E). In the cranial dura, GR1+ cells were mostly associated with the dural sinuses, and we observed evidence of intraluminal lymphatic localization, suggesting the possibility of lymphatic trafficking of myeloid cells during neuroinflammatory diseases as previously described (FIGS. 19G, 19H, and 19I).

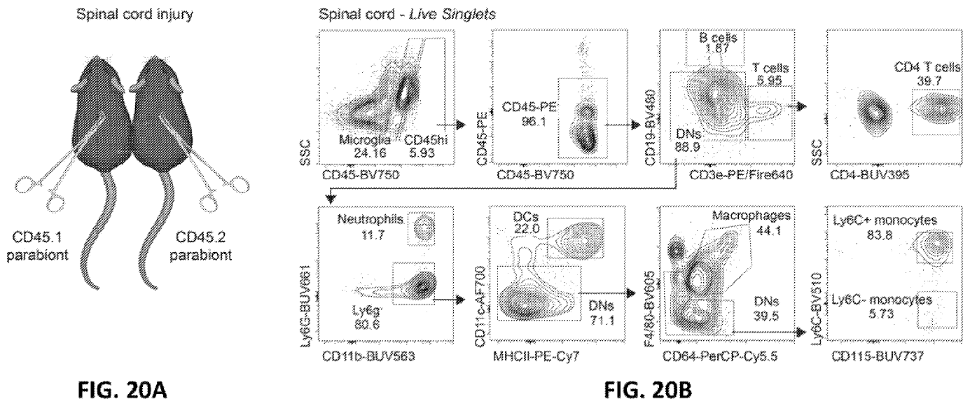

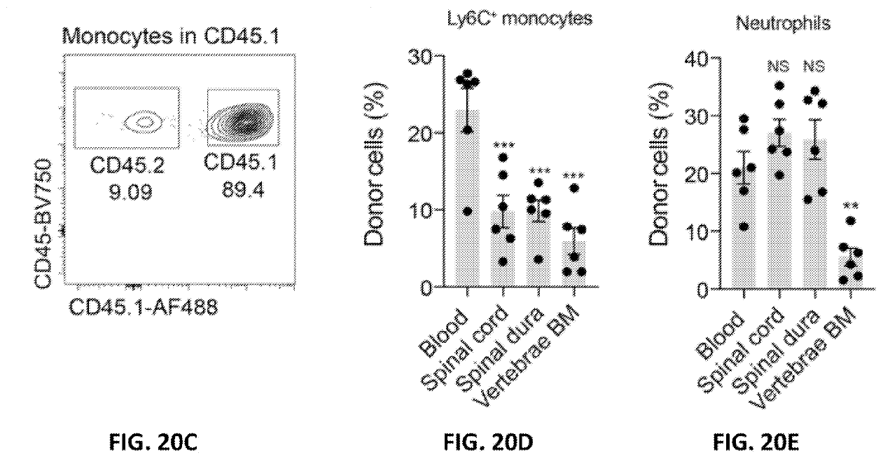

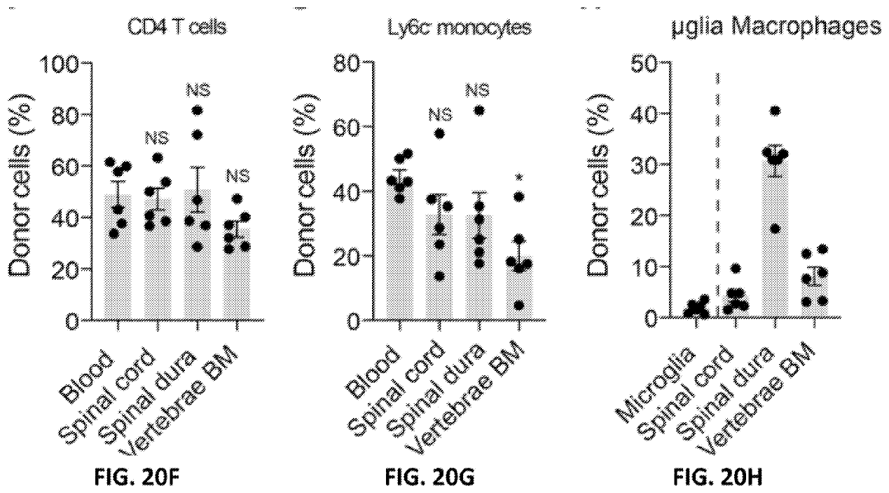

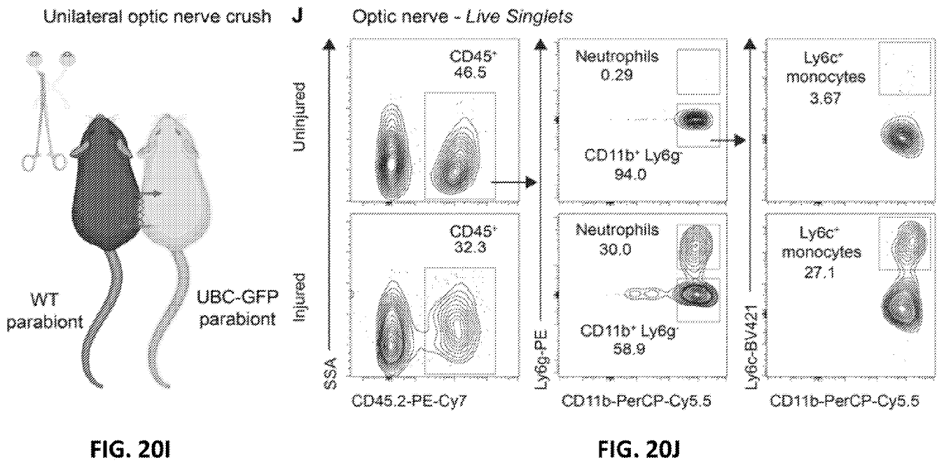

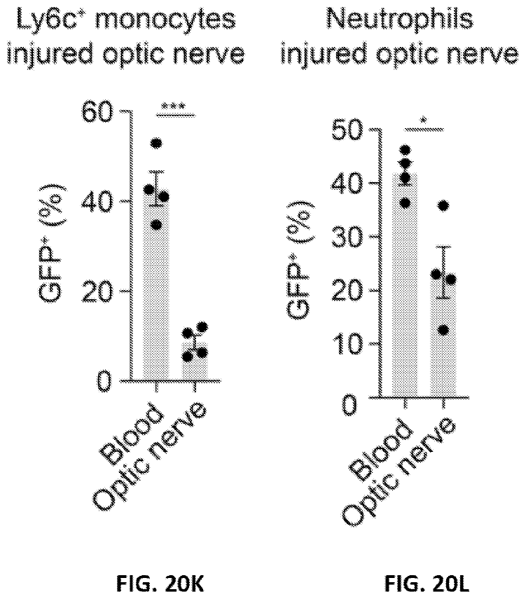

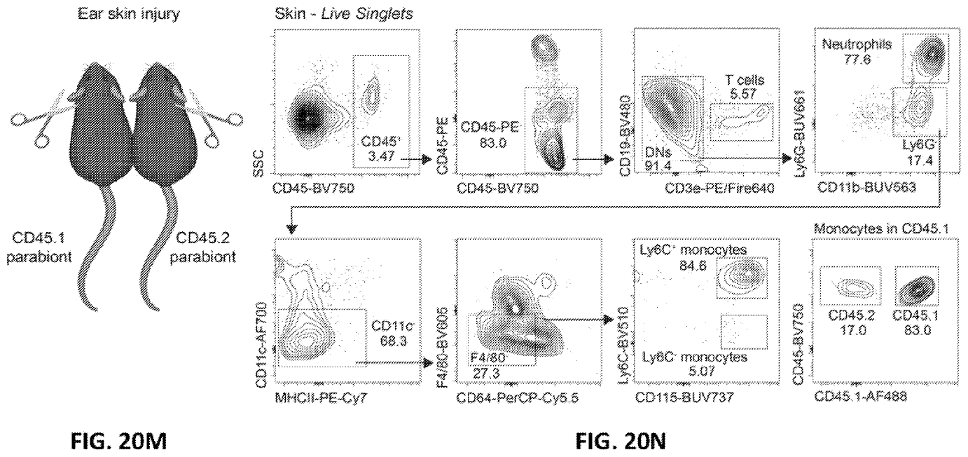

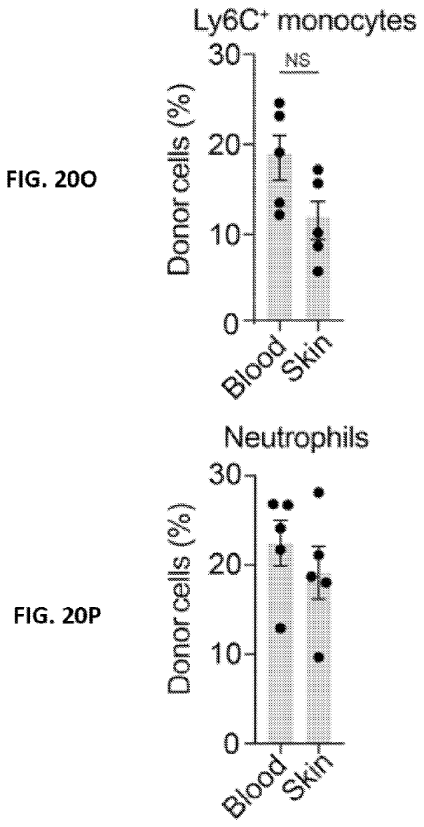

Following spinal cord injury in CD45.1-CD45.2 parabionts, CNS-infiltrating Ly6C+ monocytes (but not neutrophils, CD4 T cells, or Ly6C-monocytes) also displayed a significantly lower proportion of host-derived cells in the spinal cord and spinal dura compared to blood, suggestive of a local bone-marrow origin (FIGS. 20A, 20B, 20C, 20D, 20E, 20F, and 20G). GFP+ monocyte-derived macrophages were present, but their proportion in the injured spinal cord was low (FIG. 20H). Similar results were obtained for another CNS injury, whereby optic nerve infiltrating Ly6C+ monocytes and neutrophils following optic nerve crush in WT-UBC-GFP parabionts displayed a significantly lower proportion of donor-derived cells compared to the blood chimerism (FIGS. 20I, 20J, 20K, and 20L). Importantly, the same was not observed in a periphery injury model utilizing ear skin puncture that lacks a local bone-marrow reservoir and shows similar chimerism in the site of injury to that observed in the blood (FIGS. 20M, 20N, 20O, and 20P).

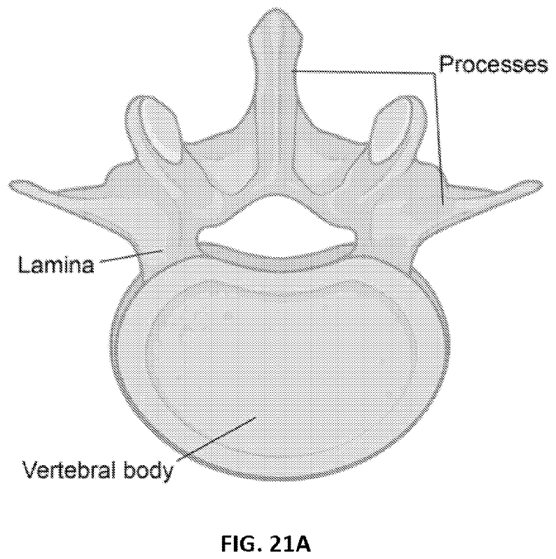

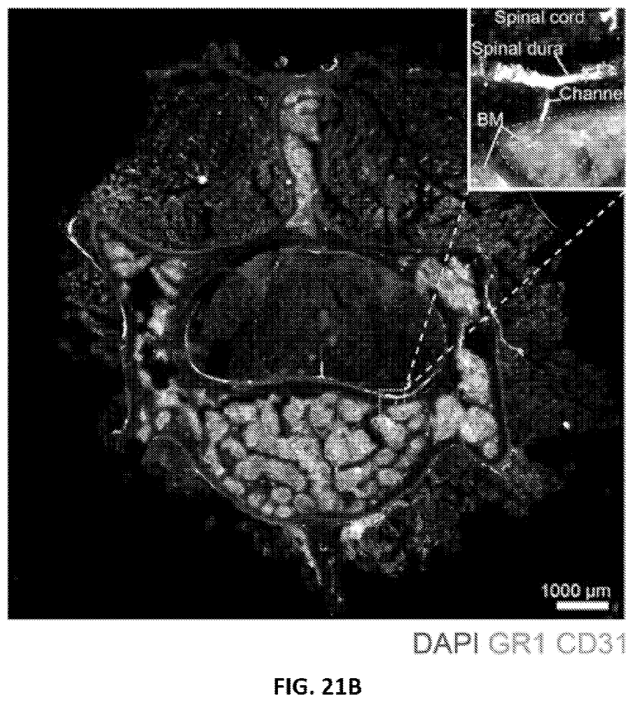

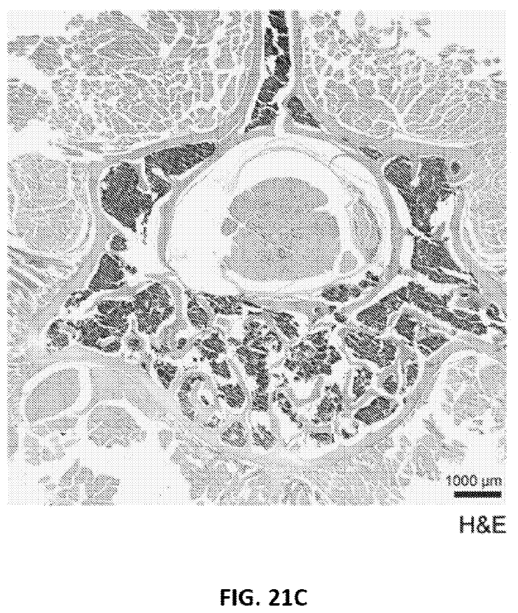

To identify a direct route allowing non-blood-derived monocytes to invade spinal cord tissue, we explored whether bone marrow channels identified for the skull were present in spinal cord vertebrae. Using H & E staining and IHC in naive and EAE mice 15 days post-induction, we observed channels directly connecting vertebrae bone marrow to spinal dura, with cells present within the channels (FIGS. 3F, 21A, 21B, and 21C). Further, we observed that spinal cord infiltrates of GR1+ cells were frequently associated with the presence of bone marrow-spinal dura channels (FIG. 3F). The exact anatomical connections of these channels are controversial, but we found that channels terminated within the dura mater and did not directly contact the CNS tissue (FIGS. 3F, 21A, 21B, and 21C). Exactly how myeloid cells traverse the underlying arachnoid and pia maters to penetrate the CNS parenchyma remains to be established, but like the blood-brain barrier, it is plausible that these barriers, consisting of tight junctions, are similarly disrupted under neuroinflammatory conditions.

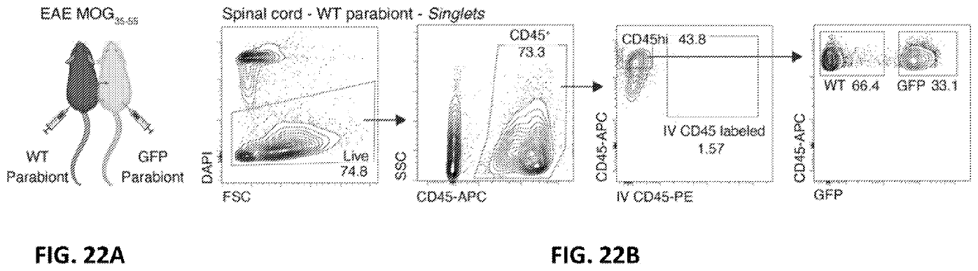

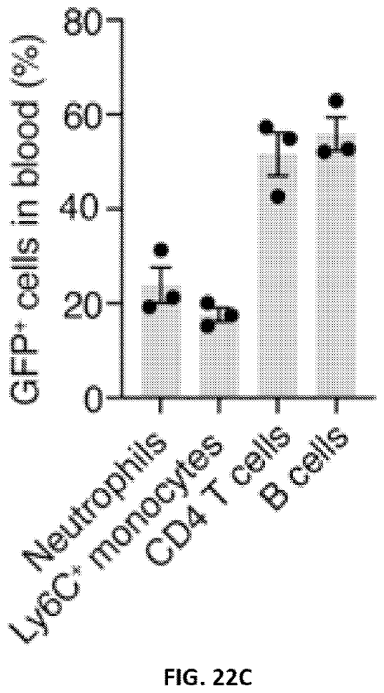

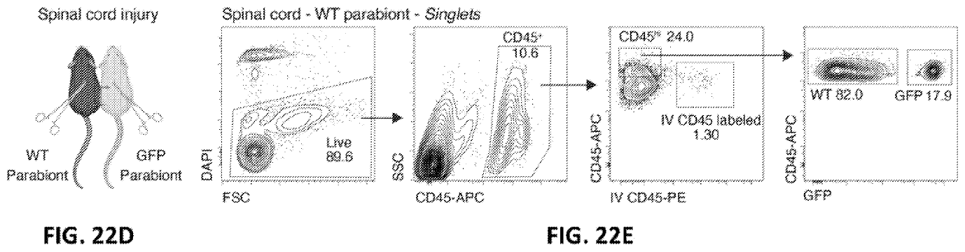

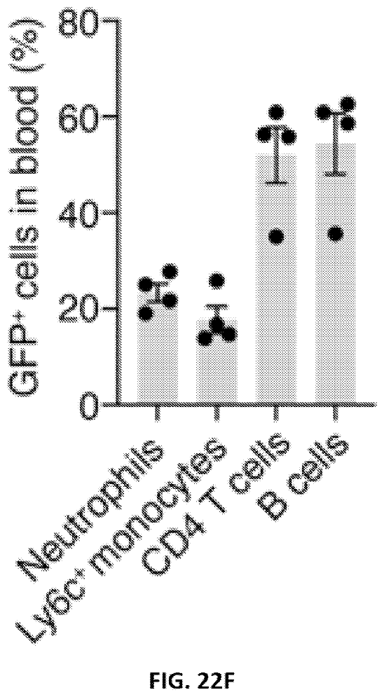

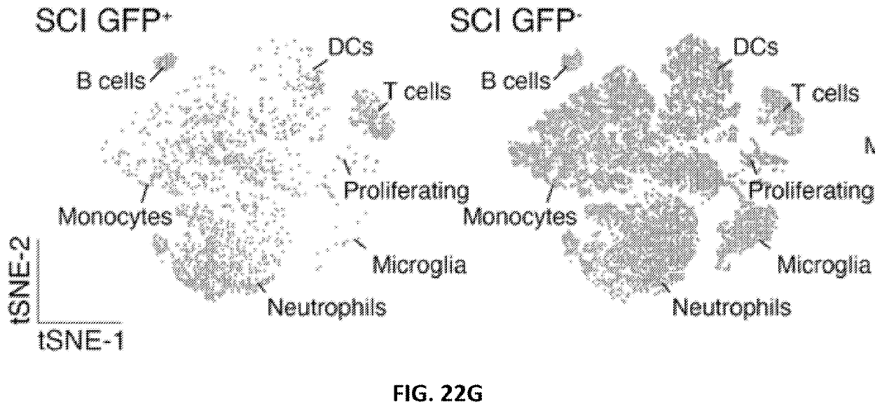

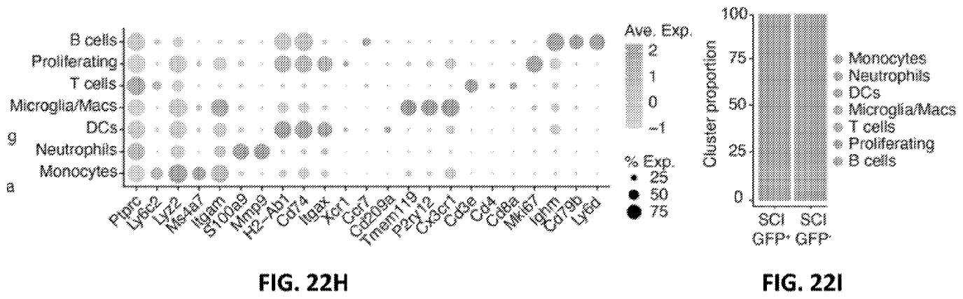

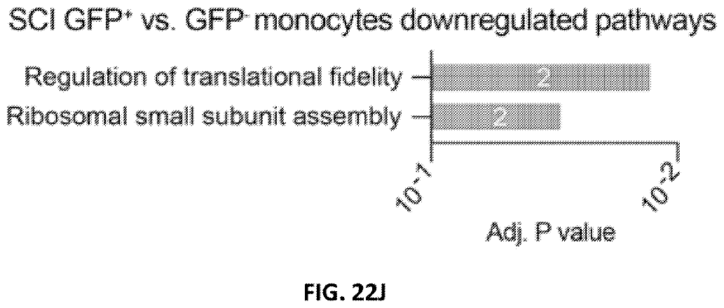

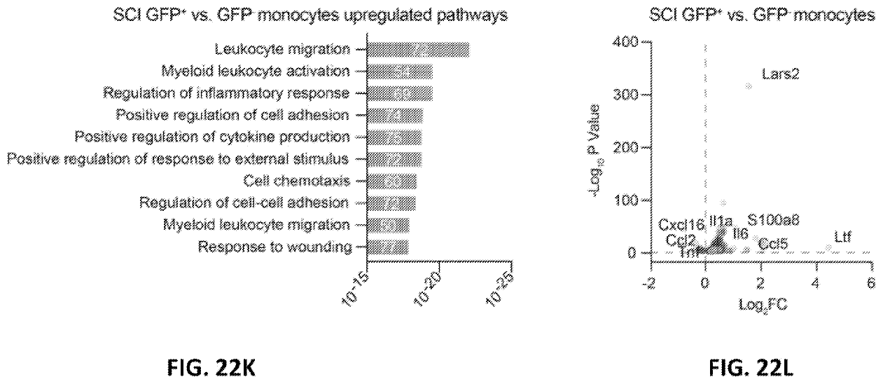

We next sought to determine whether CNS-infiltrating monocytes of different origins, blood versus bone marrow/meningeal, may have different phenotypes based on the route by which they gain access to the CNS. To explore this, we induced EAE in WT-UBC-GFP parabionts, isolated GFP+ and GFP− spinal cord-infiltrating IV CD45−CD45hi cells from WT mice 15 days post-induction, and performed scRNA-seq (FIGS. 22A, 22B, and 22C). The majority of sorted cells were monocytes, but we also obtained populations of neutrophils, dendritic cells (DCs), B cells, T cells, microglia/macrophages, and an undefined proliferating population cluster of cells from both GFP+ and GFP− populations (FIGS. 3G and 3H). In agreement with previous EAE experiments, monocytes made up the majority of the GFP− pool, consistent with a non-blood origin (FIGS. 31 and 22C). Gene ontology pathway analysis for biological processes of differentially expressed genes in GFP+vs GFP− monocytes revealed downregulated pathways involved in anion and lipid transport metabolism pathways and wound healing (FIG. 3J). Upregulated pathways were largely involved in leukocyte migration, adhesion, and T cell activation (FIG. 3K). Analysis of differentially expressed genes revealed a significant induction of diverse myeloid and lymphocyte chemokines (Ccl2, Ccl5, Cxcl9, and Cxcl6) and pro-inflammatory cytokines (IL6, Il1a, Il1b, Tnf, and Ifng) (FIG. 3L). These data suggest a potential skewed pro-inflammatory role for blood compared to bone marrow-derived monocytes in EAE, in line with the recently described pathogenic subset of CXCL10+/CXCL9+ monocytes in neuroinflammatory disease. These cells may be involved in enhanced leukocyte trafficking, for example by CCL5-CXCL9- and CXCL16-mediated T cell recruitment, and proinflammatory cytokine production, which are critical factors in EAE pathogenesis.

Notably, scRNA-seq analysis of infiltrating IV CD45−CD45hi GFP+ versus GFP− monocytes from the spinal cord of WT parabionts three days post spinal cord injury, revealed a similar upregulation of pathways involved in leukocyte migration and activation, with several conserved upregulated chemokines and cytokines in GFP+ blood-derived monocytes (FIG. 22D, 22E, 22F, 22G, 22H, 22I, 22J, 22K, and 22L). Thus, there may be differential roles in blood versus bone-marrow-derived monocytes in diverse CNS injury and inflammatory conditions, perhaps similar to those observed for choroid plexus compared to blood trafficking during spinal cord injury.

### Example 2

To demonstrate that CSF interfaces with CNS bone marrow niches, the following experiments are conducted.

We hypothesized that local CNS cues, contained in CSF, could instruct myeloid cell recruitment to the meninges. Efflux of molecules from the brain is achieved via the blood-brain barrier 9, 10 and via CSF perfusion through the brain in a process termed ‘glymphatic clearance’11. Through a glymphatic mechanism, brain-derived molecules are continuously cleared via CSF, efflux to the parasagittal dura mater, and subsequently drain through meningeal lymphatic vessels, enabling immune surveillance of the CNS from distant sites 4, 12. Recent studies demonstrated that the skull bone marrow also connects directly to the underlying dura through ossified vascular channels 13-15. Although these channels have been previously described to allow myeloid and lymphoid cell migration from the skull bone marrow to the dura, we speculated that these pathways might be bi-directional, allowing bone marrow direct access to the CSF. Here we show that CSF accesses skull bone marrow niches, where it regulates myelopoiesis and egress to meninges in physiology and pathology.

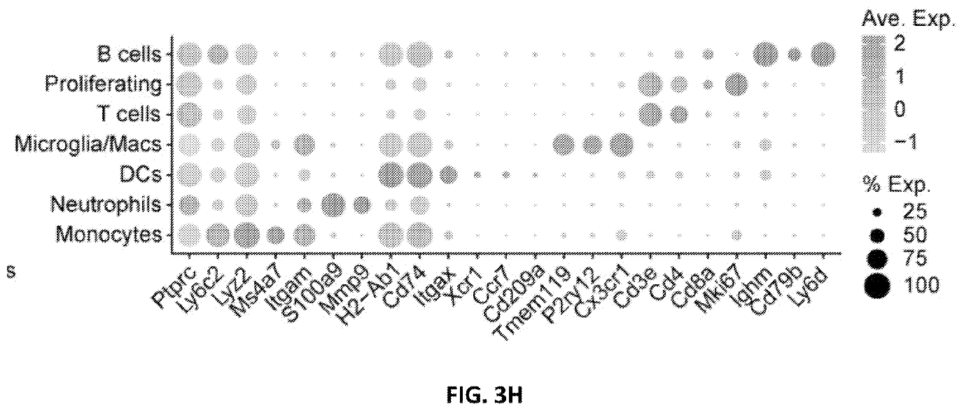

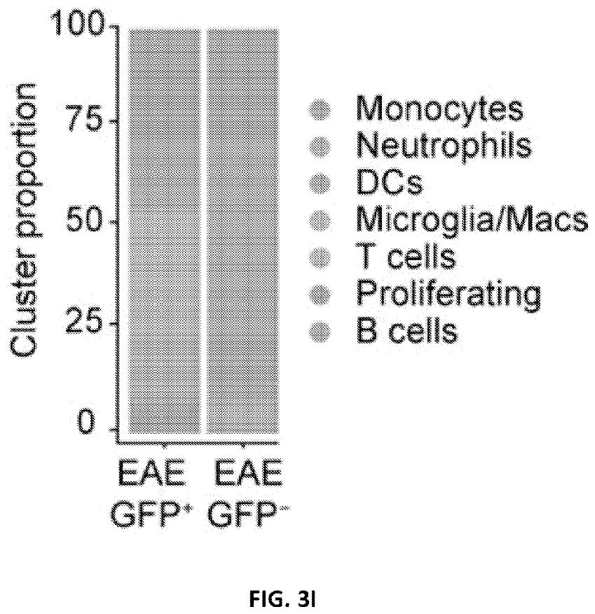

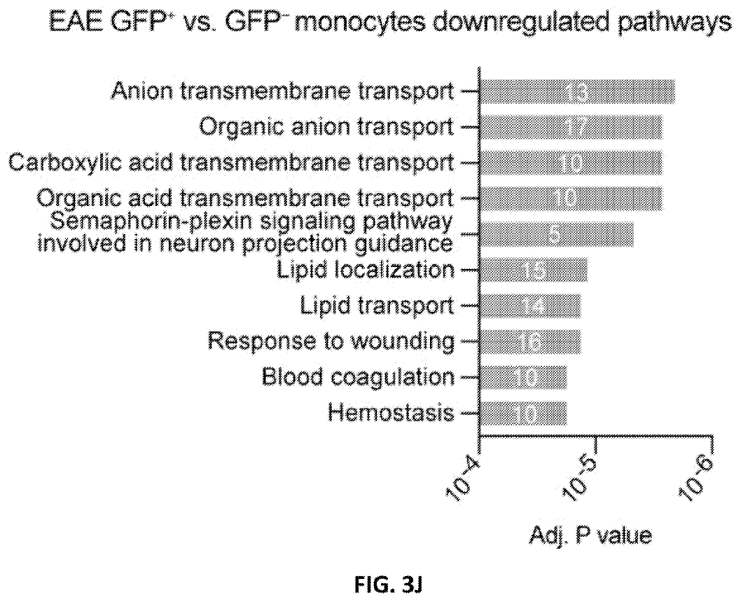

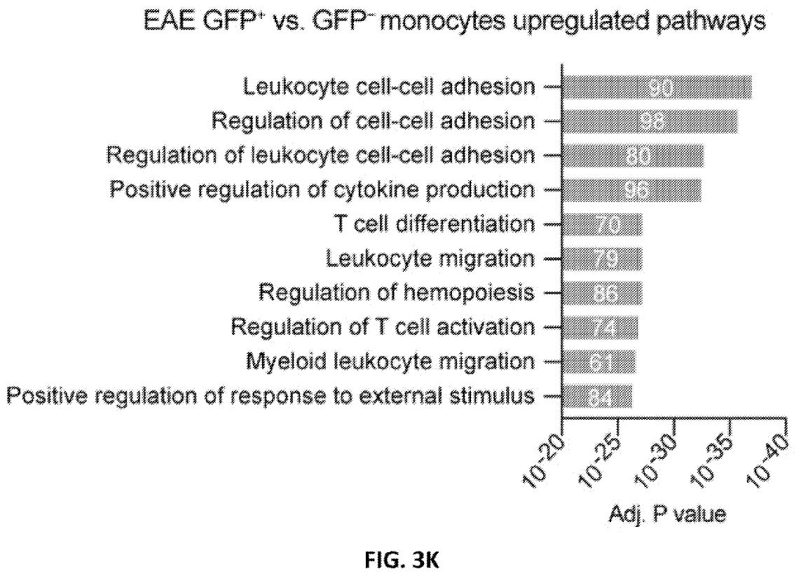

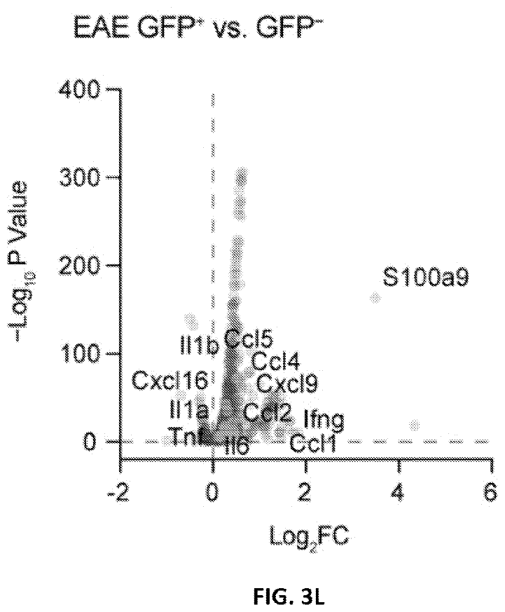

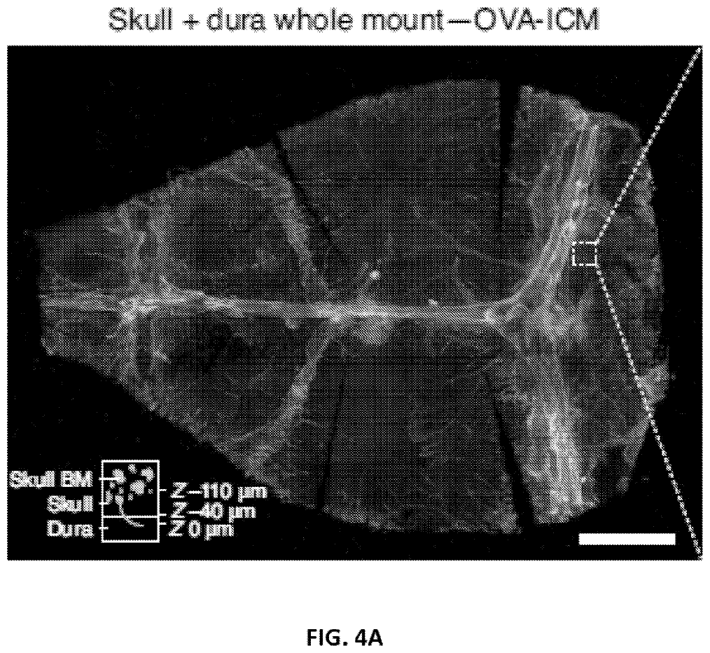

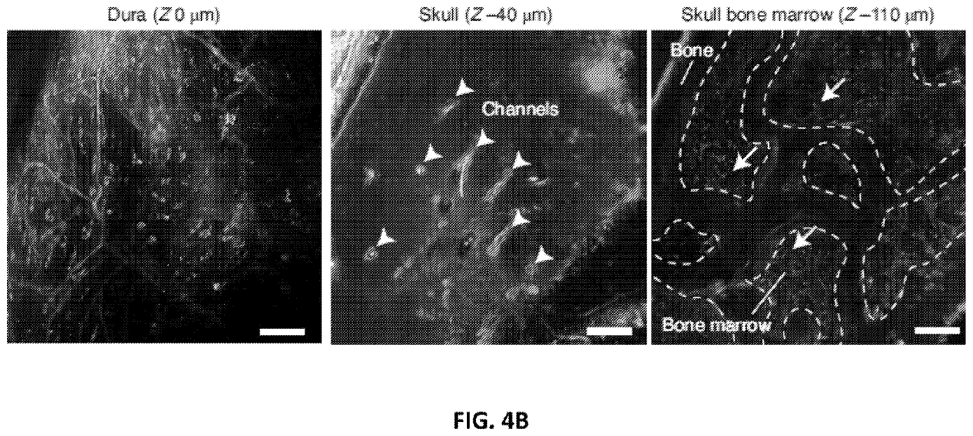

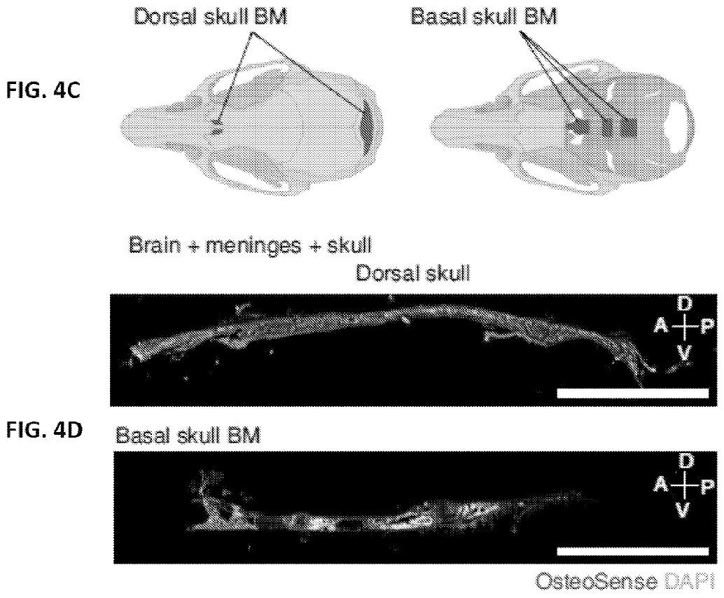

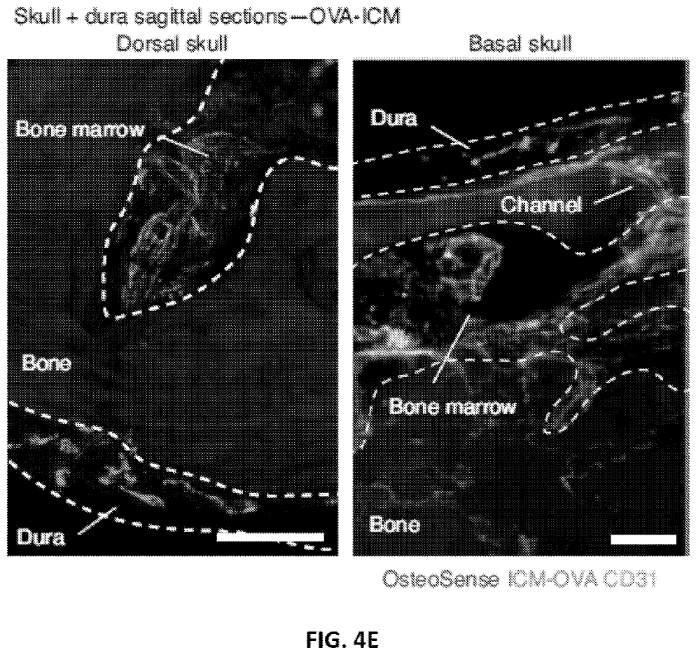

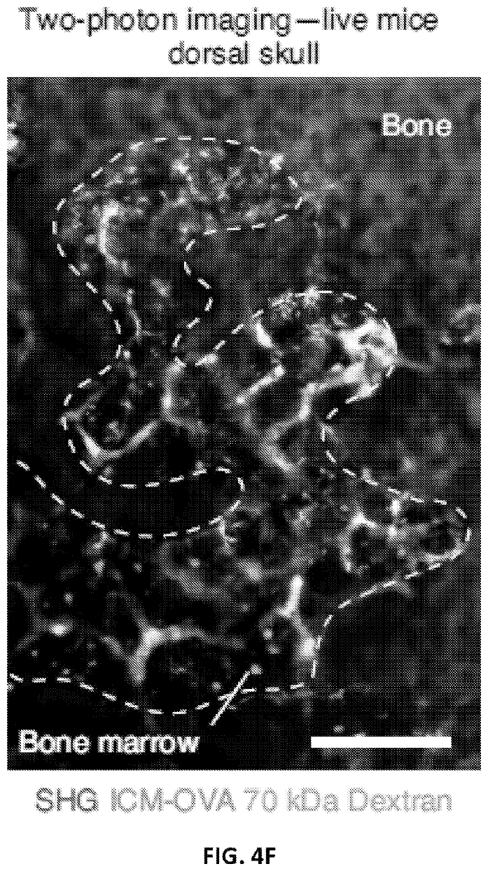

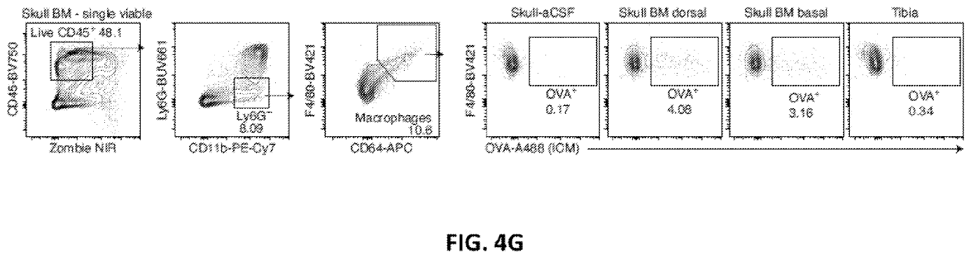

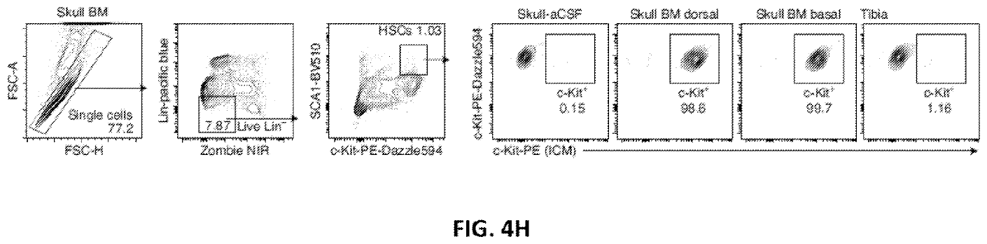

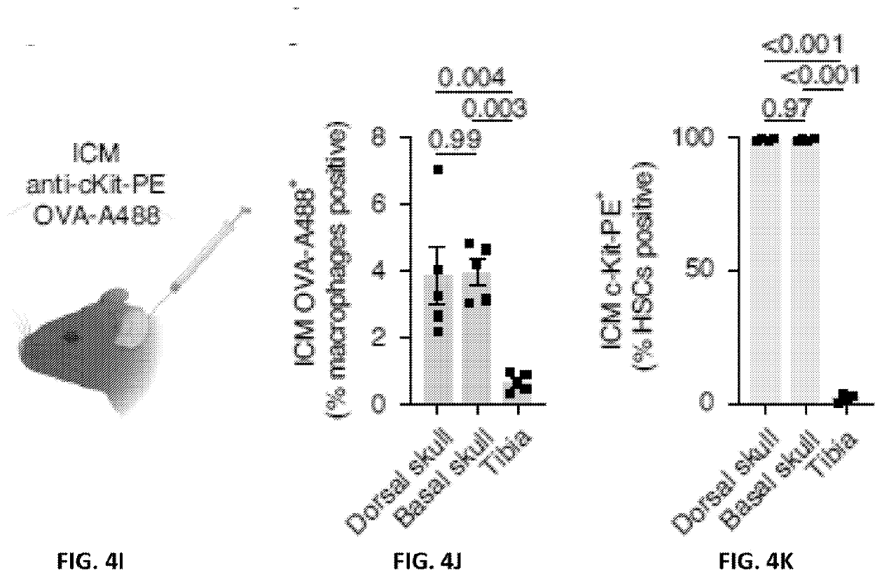

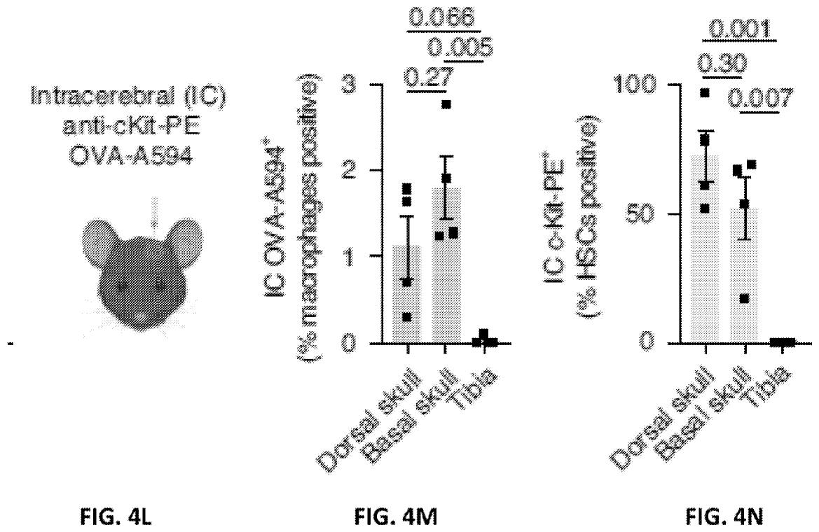

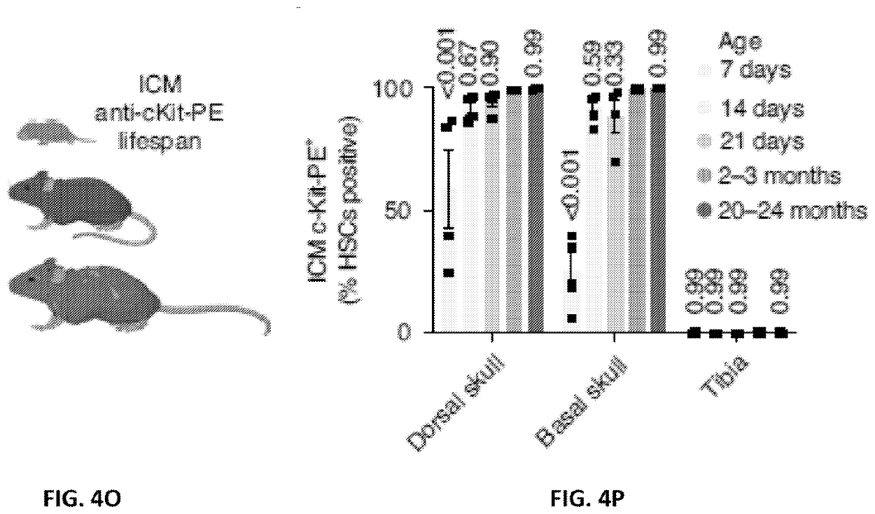

To test the possibility that CSF interfaces with CNS bone marrow niches, we injected fluorescent ovalbumin (OVA, ˜45 kDa) into CSF via the cisterna magna (intra-cisterna magna (ICM)) and examined its efflux to skull bone marrow after 1 hour. Whole-mounted decalcified and cleared skullcaps with underlying dura revealed uptake of tracer along dural sinuses (FIGS. 4A and 4B), as previously described. Three-dimensional reconstructions of skull bone marrow regions also showed tracer along perivascular conduits within ossified channels and within the bone marrow niche (FIGS. 4A and 4B).

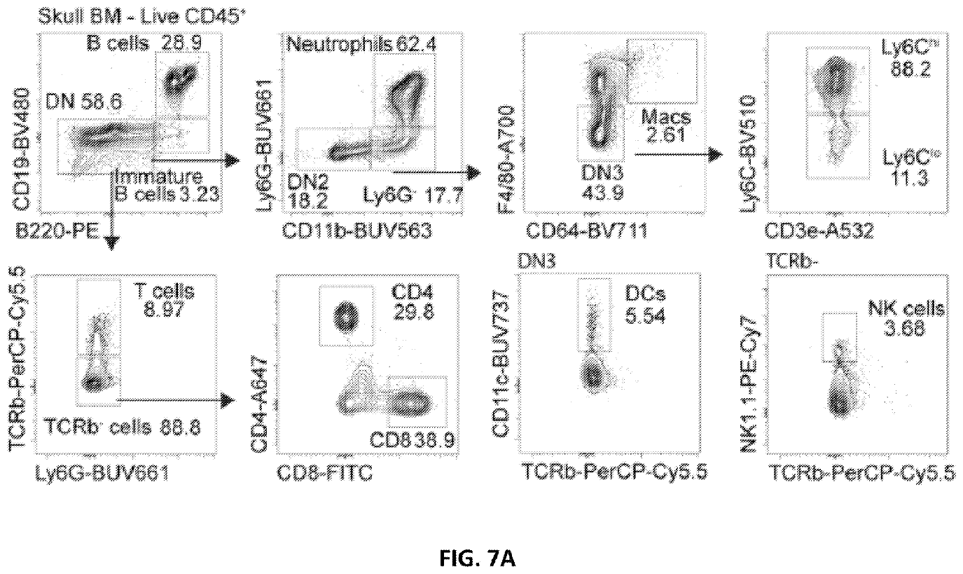

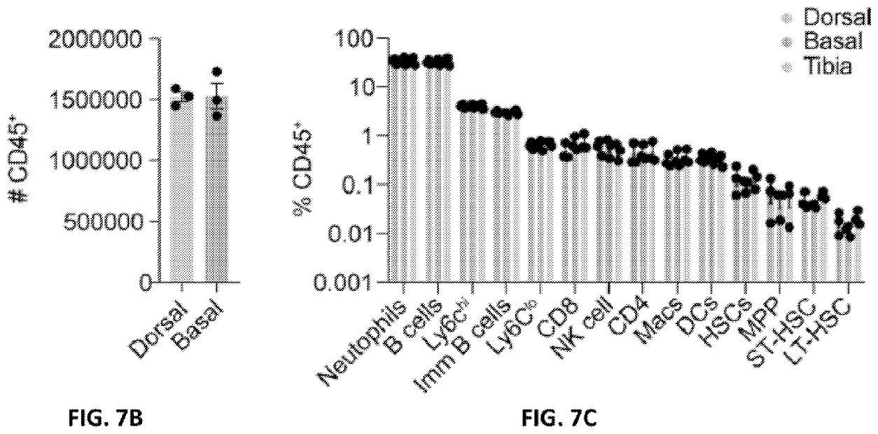

Although the dorsal aspect of the skull bone marrow contains ossified channels that provide myeloid and lymphoid populations directly to the underlying dura, whether similar anatomical structures are present in the skull base is unclear. Examination of the skull base revealed enriched pockets of marrow (FIGS. 4C and 4D) with an equivalent array of the stem, progenitor, myeloid and lymphoid populations (FIG. 7A, 7B, 7C). Assessment of skull base bone marrow niches demonstrated the conserved presence of channels and similar perivascular conduits of CSF tracer efflux as those observed in the dorsal skull (FIG. 4E). To exclude any potential contribution of postmortem artifacts, we employed intravital two-photon microscopy and observed the CSF tracer in the dorsal skull bone marrow niche 30 minutes after ICM injection (FIG. 4F).

To confirm that CSF interfaces with cells in the skull bone marrow, we assessed the accumulation of OVA signal in macrophages and observed uptake in both dorsal and basal skull niches after ICM injection (FIGS. 4G, 4I, and 4J). To demonstrate that CSF interacts with other cells within the bone marrow niche, we assessed the labeling of hematopoietic stem cells (HSCs) (Lin-Sca1+c-Kit+(LSK)) by flow cytometry of young-adult mice after ICM injection of an anti-c-Kit antibody. One hour after ICM delivery, 99% of LSK cells in the skull bone marrow were labeled with the antibody (FIGS. 4H, 4I, and 4K). Intracerebral injection of OVA and anti-c-Kit antibody also labeled macrophages and HSCs, respectively, suggesting that parenchymal solutes traffic into the CSF, as previously described, and subsequently access skull bone marrow (FIGS. 4L, 4M, and 4N). Notably, 1 hour after either ICM or intracerebral (IC) injection, neither c-Kit-labeled nor OVA-labeled cells were found in peripheral tibial bone marrow, confirming that this CSF access reflects direct CSF-to-skull bone marrow communication rather than peripheral blood recirculation (FIGS. 4I, 4J, 4K, 4L, 4M, and 4N).

Paravascular glymphatic fluid flow and meningeal lymphatic drainage of CSF change throughout the lifespan. We, therefore, wondered whether CSF access to skull bone marrow similarly changes throughout postnatal development and aging because of altered CSF dynamics. Surprisingly, no major changes in CSF-skull bone marrow accessibility were observed beyond the second postnatal week (FIGS. 4O and 4P), suggesting that CSF-derived factors have the potential to shape skull bone marrow niches throughout the entire lifespan.

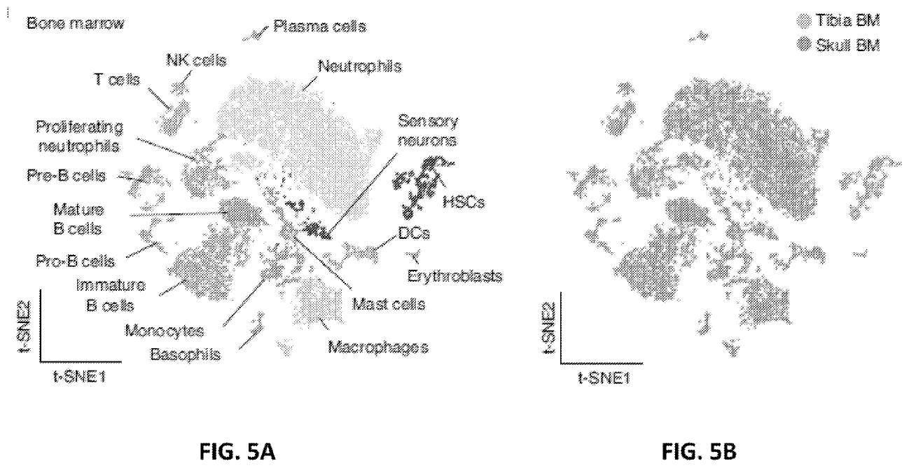

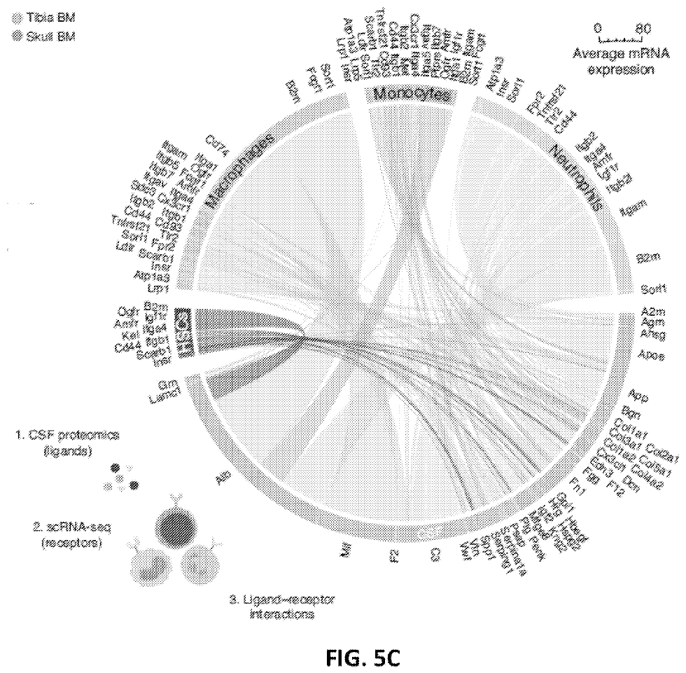

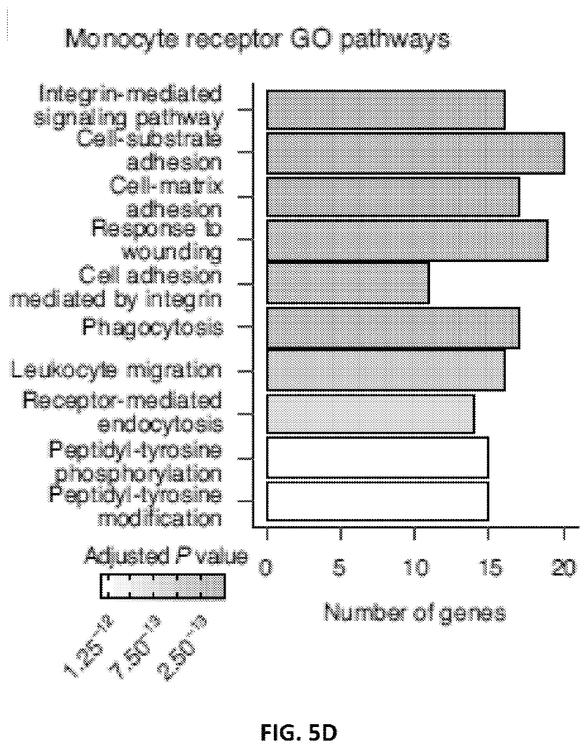

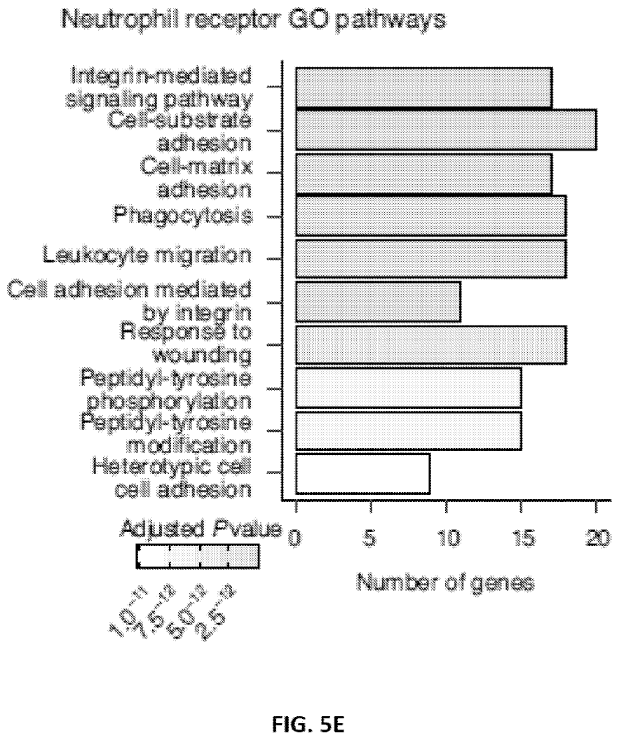

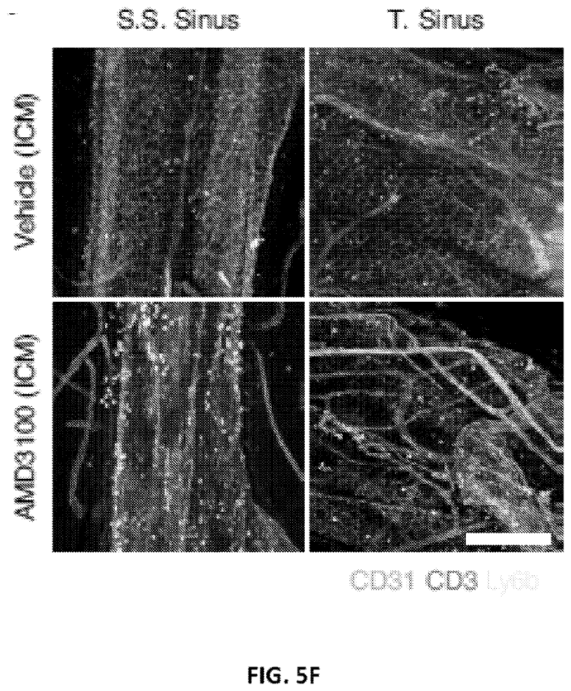

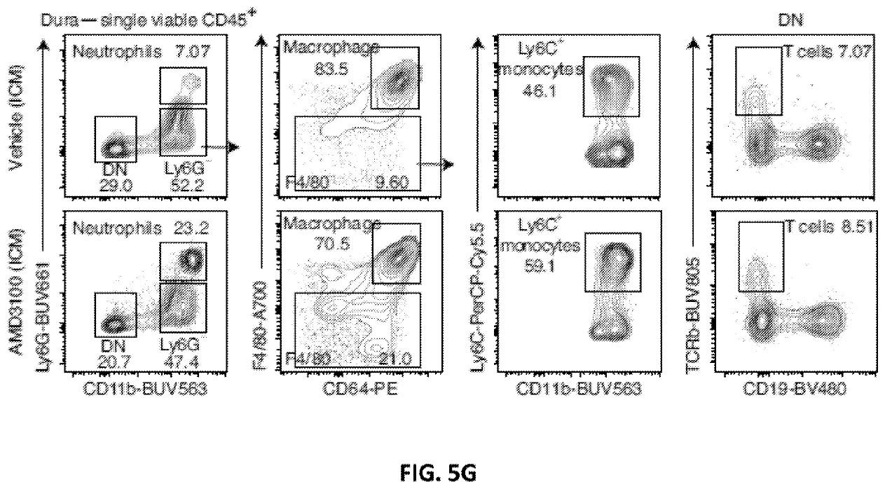

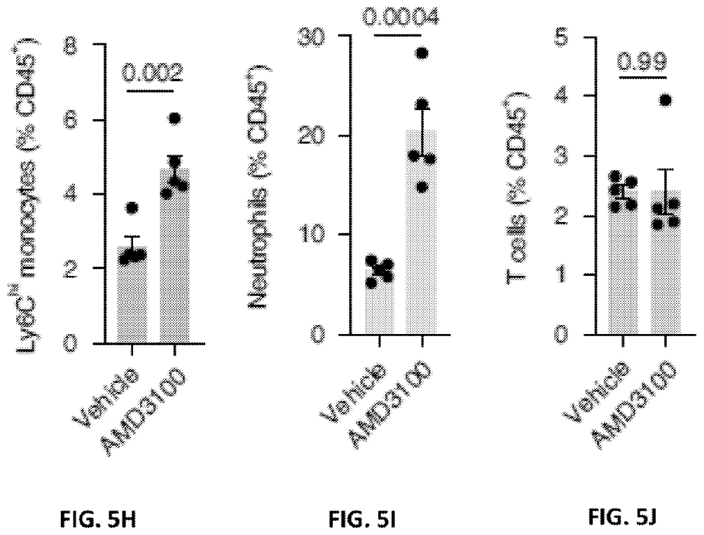

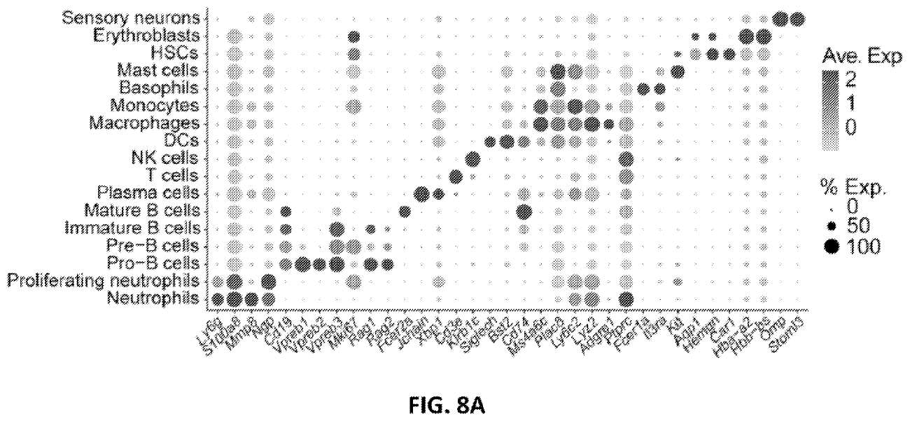

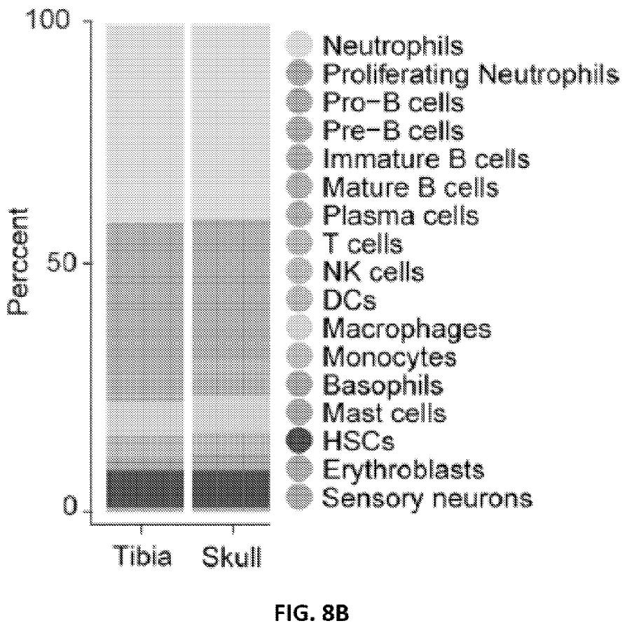

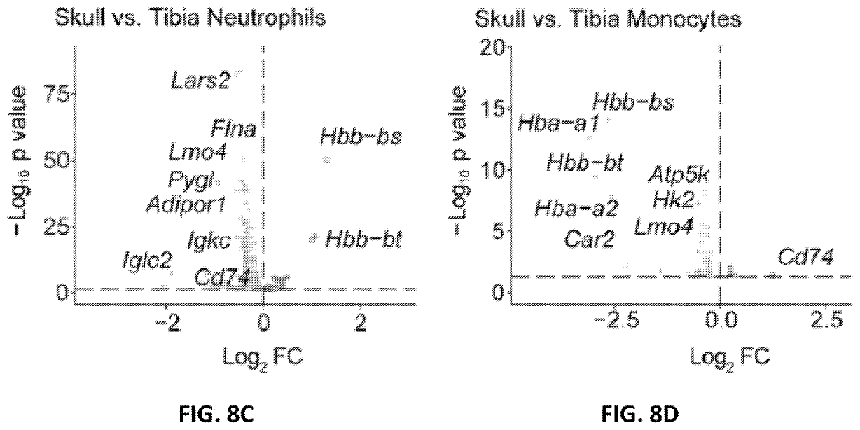

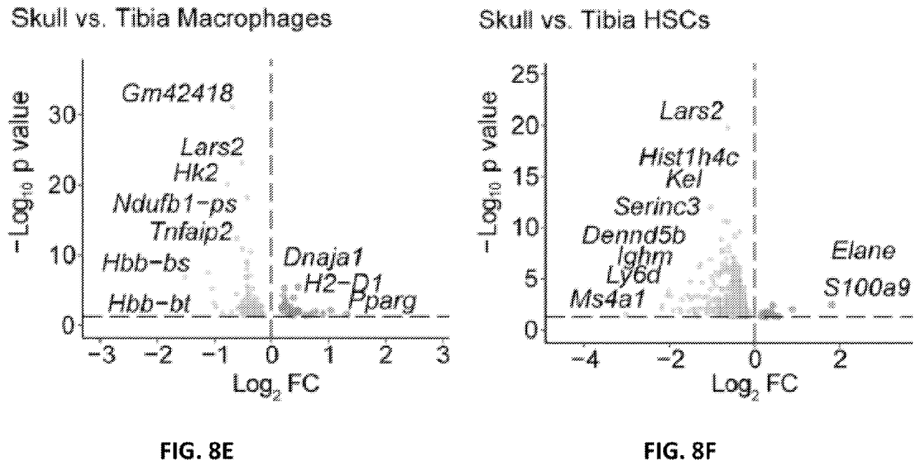

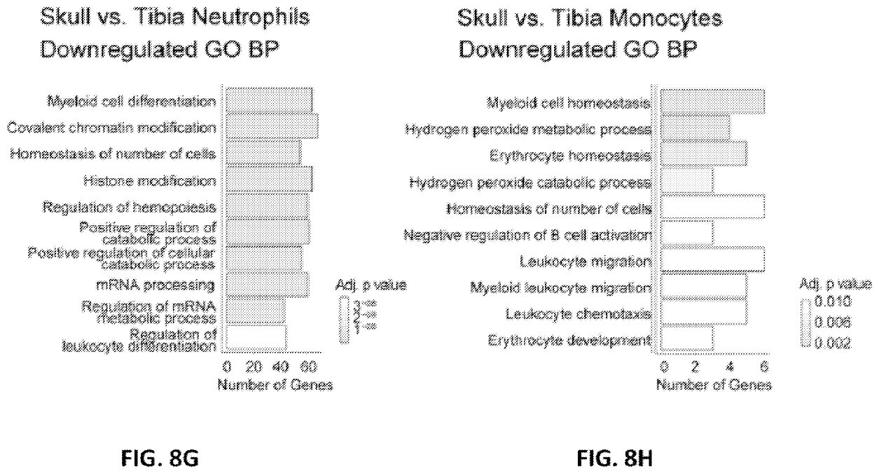

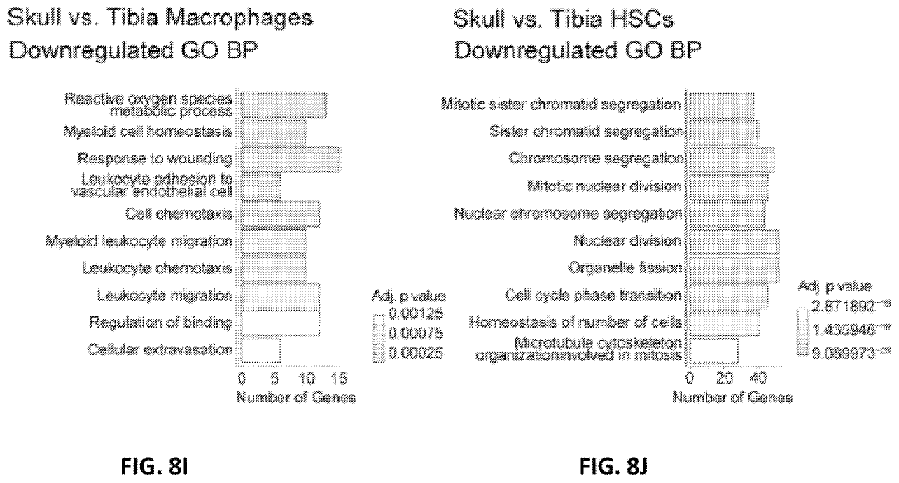

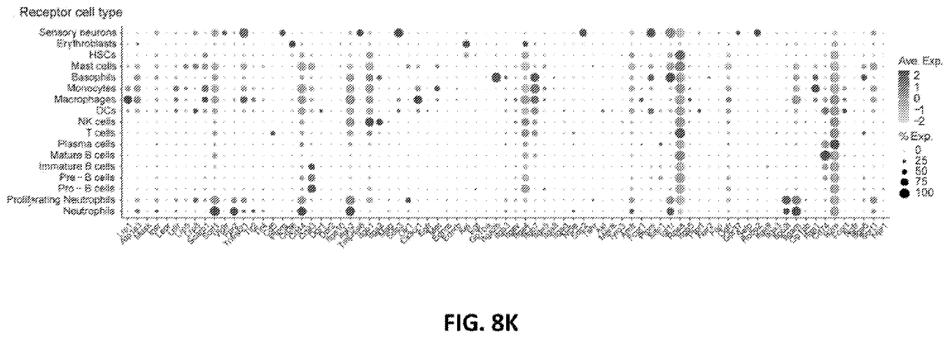

CSF composition shapes neurogenic niches during CNS development through direct ligand-receptor signaling 20. Given that CSF directly accesses skull bone marrow, we asked whether exposure to CSF affords the skull marrow niche a unique phenotype. To this end, we performed single-cell RNA sequencing (scRNA-seq) of the dorsal skull and tibial bone marrow from young-adult mice. Phenotypic analysis revealed expected bone marrow populations 21, including HSCs, monocytes, neutrophils, basophils, mast cells, erythroblasts, sensory neurons, dendritic cells, natural killer cells, T cells, and B cell developmental trajectories (FIGS. 5A, 5B, and 8A). Although there were no major differences in the cellular composition of skull versus tibia bone marrow niche, differences were observed within neutrophil, monocyte, macrophage, and HSC populations, including downregulation of genes involved in proliferation in HSCs, reactive oxygen species production in monocytes and macrophages and myeloid cell differentiation in neutrophils (FIGS. 8B, 8C, 8D, 8E, 8F, 8G, 8H, 8I, and 8J). We, therefore, wanted to further explore the nature of steady state signaling between CSF and skull bone marrow to determine if CSF factors might modify bone marrow physiology. We performed a proteomic analysis of CSF from young-adult mice, and, leveraging our scRNA-seq dataset, uncovered a set of potential ligand-receptor interactions between CSF proteins and diverse cells in the skull bone marrow (FIGS. 5C and 8K).

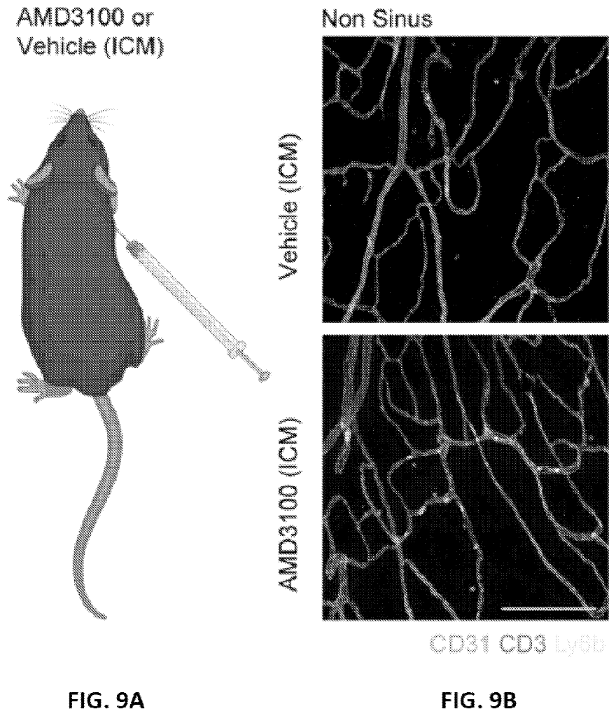

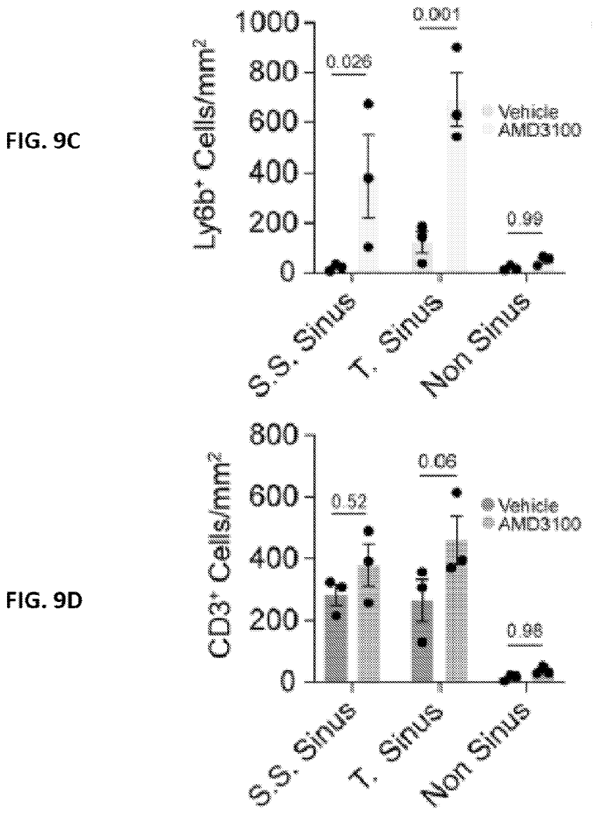

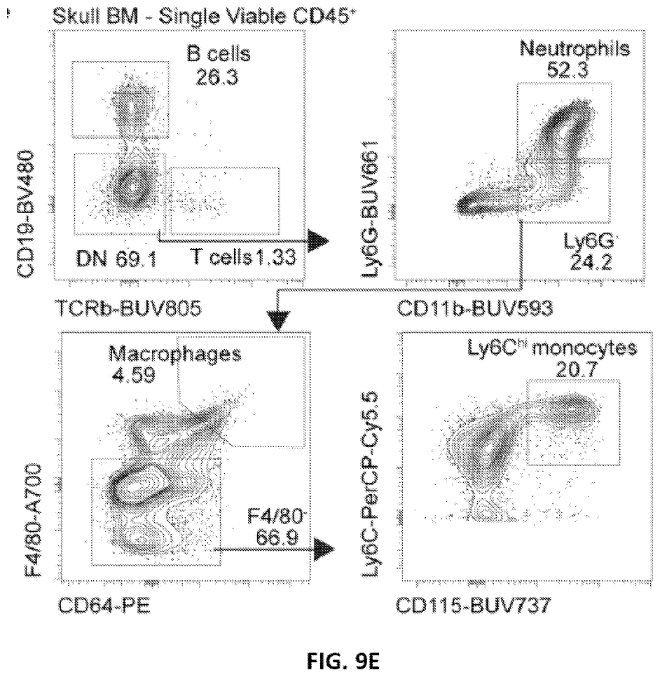

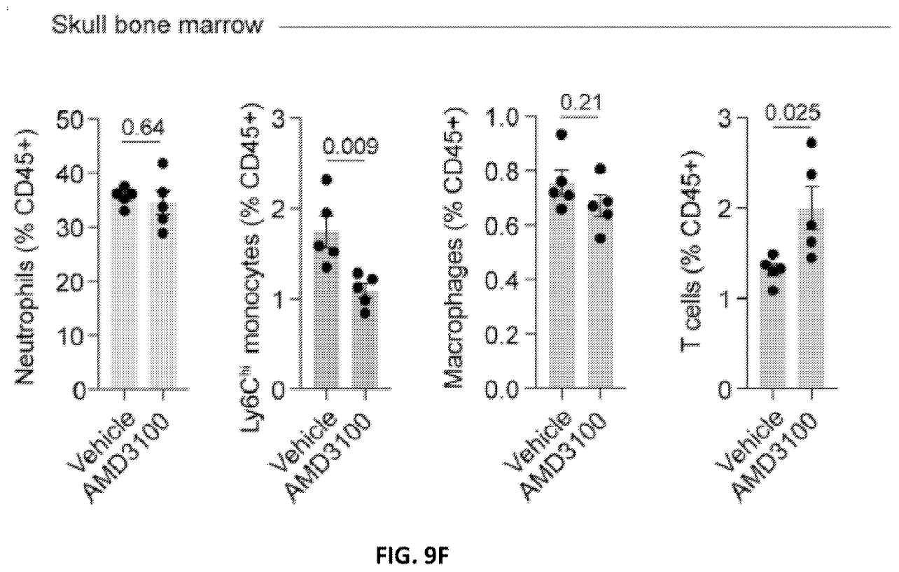

Examining Gene Ontology pathways of these ligand-receptor interactions in monocytes and neutrophils revealed signaling mechanisms enriched for leukocyte migration, cell adhesion, and phagocytosis (FIGS. 5D and 5E). These enriched pathways suggested that CSF-derived factors could instruct the mobilization and recruitment of myeloid cells from the skull bone marrow. To test this, we injected AMD3100, a CXCR4 antagonist, into the CSF and assessed monocyte and neutrophil egress from the skull bone marrow to the dura. By immunostaining, we found that Ly6b+ monocytes and neutrophils in the dura were significantly enriched after AMD3100 administration and preferentially clustered at nearby sinuses underlying skull bone marrow niches (FIGS. 5F, 9A, 9B, 9C, and 9D). Using flow cytometry, we confirmed a significant increase in Ly6Chi monocytes and neutrophils in the dura, along with a concomitant decrease in overlying skull bone marrow, suggesting local bone marrow egress rather than dural proliferation (FIGS. 5G, 5H, 5I, 9E, and 9F). Notably, we did not detect a change in dural T cells (FIG. 5J) or any changes in tibial monocytes or neutrophils (FIG. 9G), consistent with the notion of T cell trafficking through blood vasculature, whereas myeloid cells migrate directly from skull bone marrow niches. These results confirmed that myeloid cells can egress from the skull bone marrow in response to CSF-derived cues, suggesting an ability to dynamically respond to altered brain states.

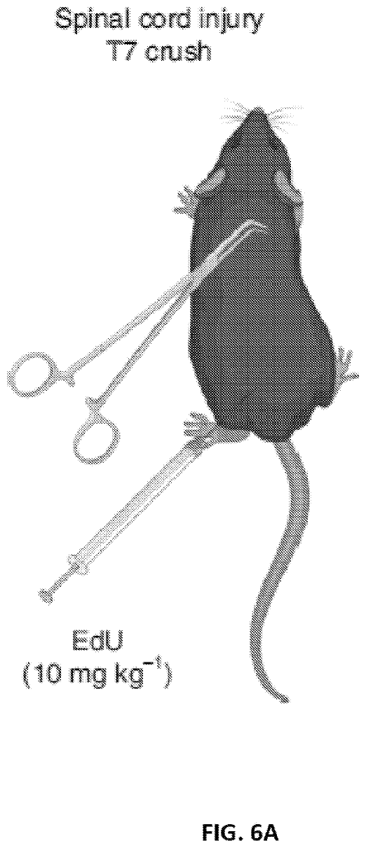

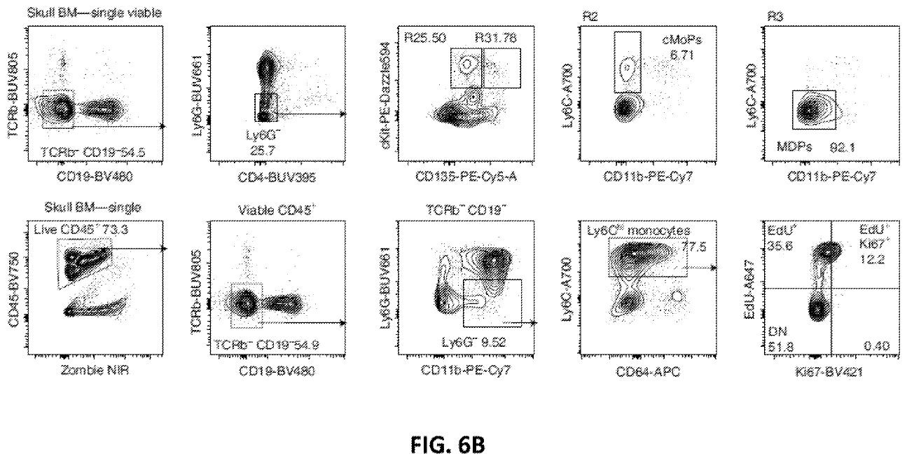

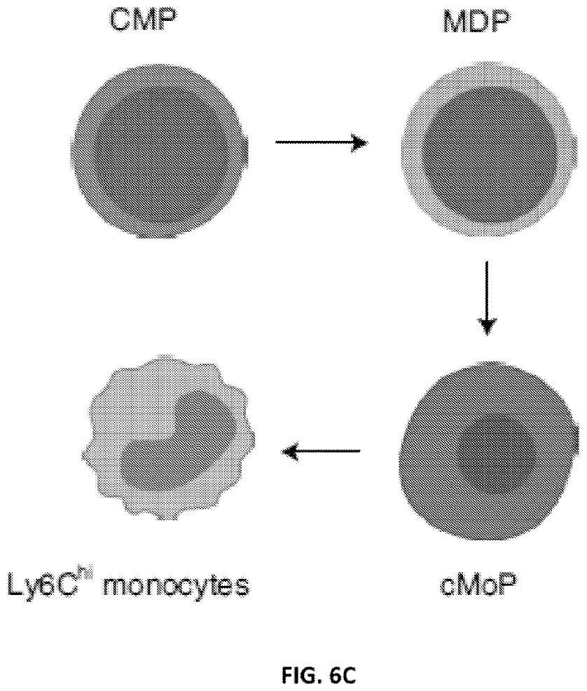

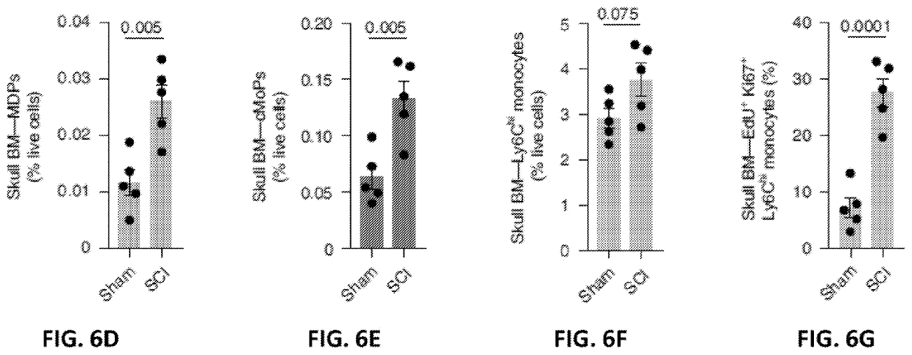

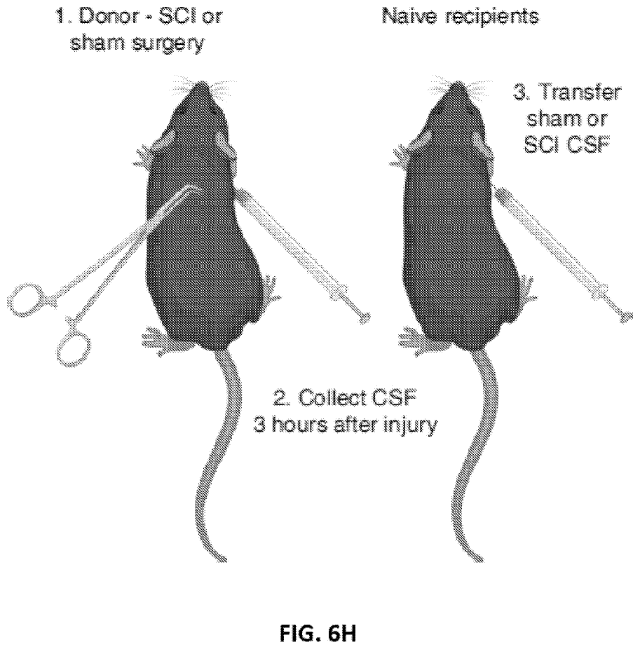

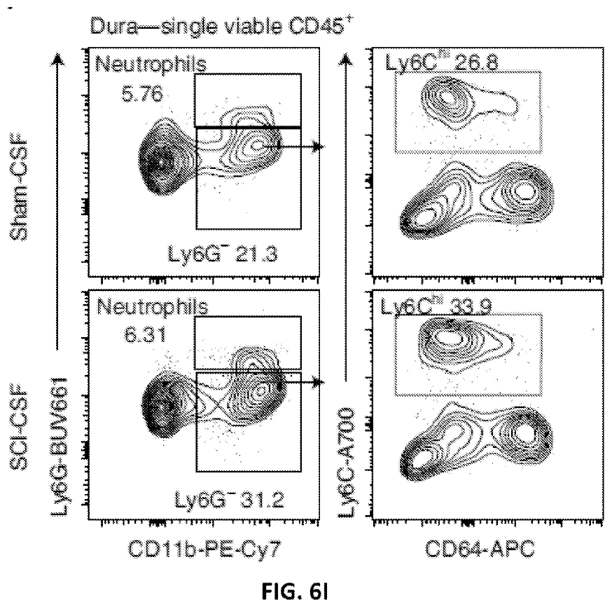

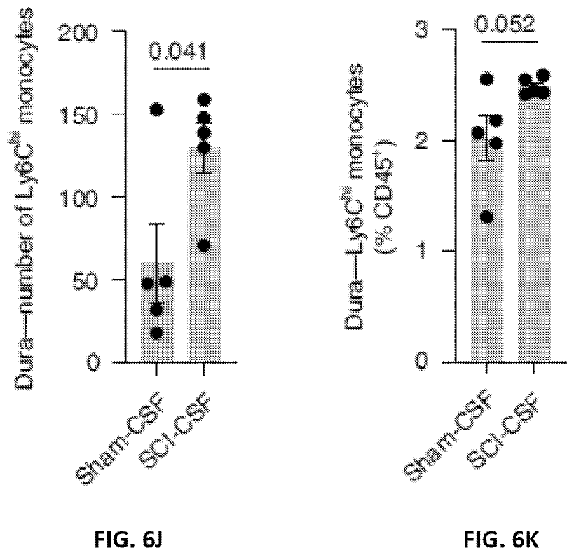

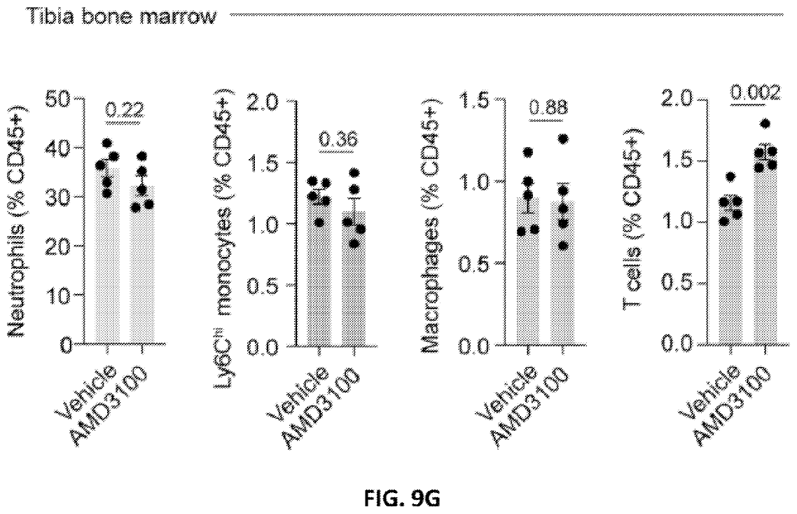

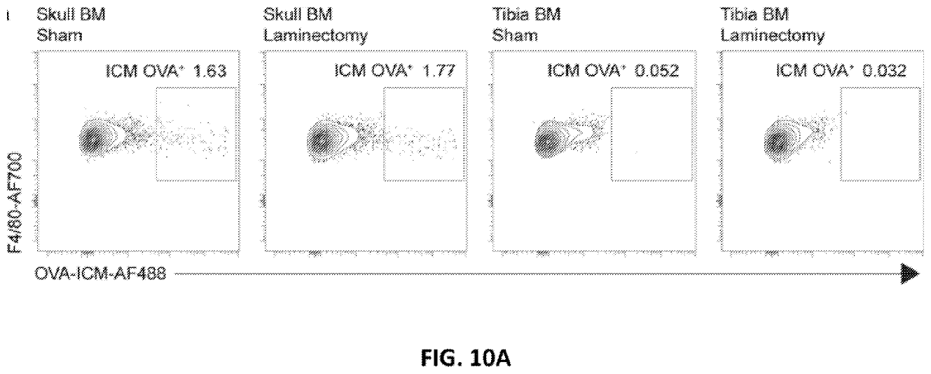

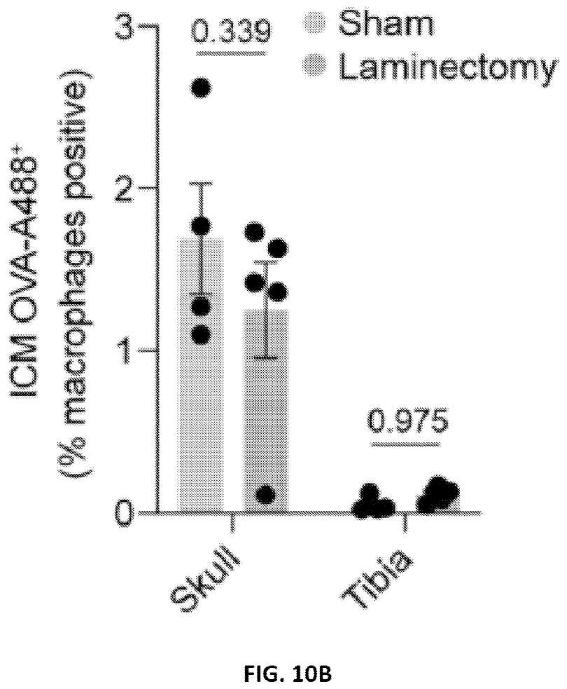

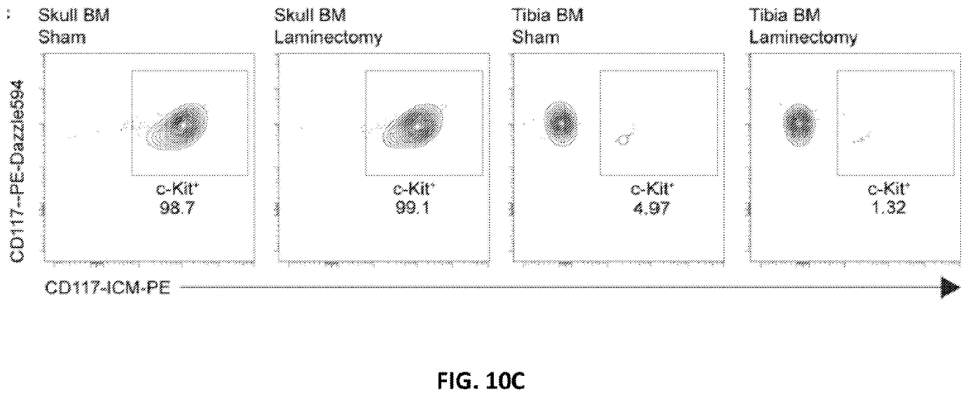

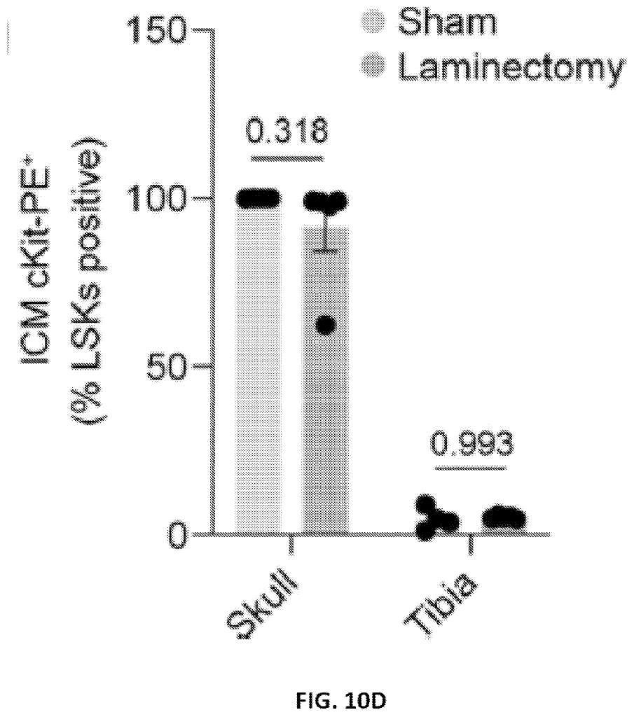

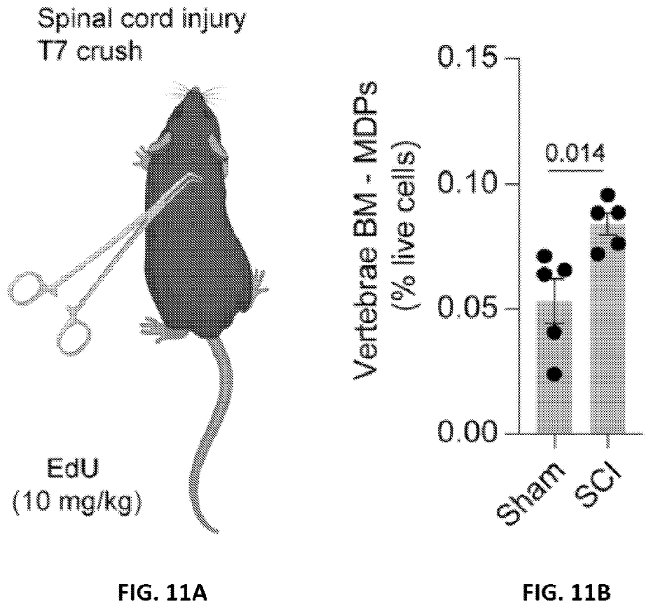

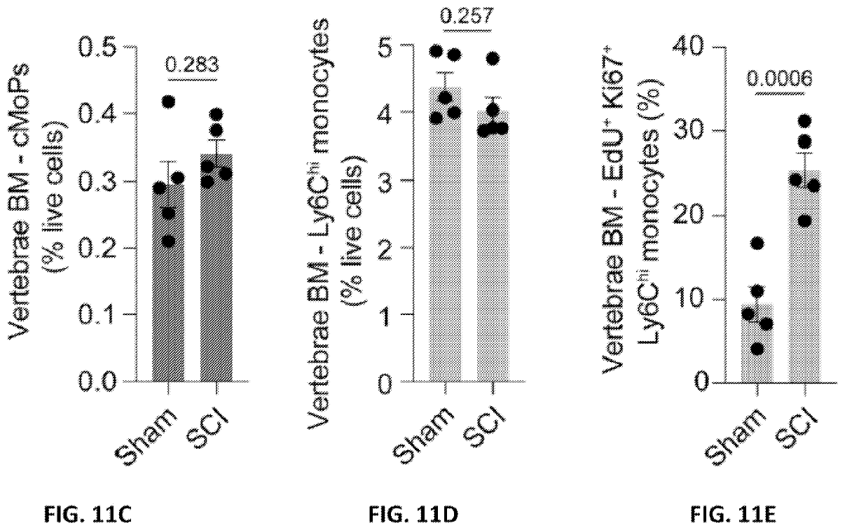

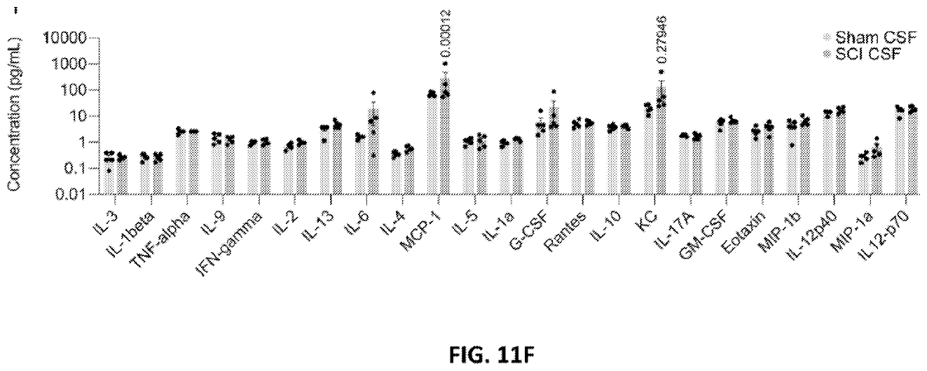

We next asked whether physiological changes in CSF composition—for example, after CNS injury-could instruct skull bone marrow mobilization. To test this, we performed a spinal cord crush injury-during which a laminectomy is performed and the spinal cord meninges are left intact, thus preventing CSF leakage—in young-adult mice and asked whether a distant CNS injury could be sensed by the skull bone marrow. Notably, laminectomy did not impair CSF access to the skull bone marrow niche (FIGS. 10A, 10B, 10C, and 10D). Three hours after spinal cord injury, vertebrae and skull bone marrow were collected, and myeloid progenitors were assessed. As expected, vertebrae bone marrow was highly activated after the injury, characterized by elevated numbers of proliferating monocytes (FIGS. 1IA, 11B, 11C, 11D, and 11E). Interestingly, remote skull bone marrow niches also showed a substantial increase in the percent of monocyte dendritic precursors (MDPs), common monocyte progenitors (cMoPs), and proliferating monocytes compared to sham controls (FIGS. 6A, 6B, 6C, 6D, 6E, 6F, and 6G). Additionally, an assessment of CSF composition after spinal cord injury revealed elevated levels of the monocyte chemoattractant CCL2 (MCP-1) (FIG. 11F). These results suggest that CSF carries cues to neighboring bone marrow niches to induce myelopoiesis and provide myeloid cells to underlying dura or brain tissue after CNS insults.

To determine whether the skull bone marrow response after spinal cord injury is indeed mediated by CSF-contained signals, and to confirm that this is sufficient to promote myeloid cell trafficking, we performed CSF transfer experiments. Sham (that included laminectomy) or spinal cord injury was performed on young-adult mice, and, 3 hours later, their CSF was collected and transferred into naive mice (FIG. 6H). Six hours after CSF transfer, we observed a significant increase in the number of monocytes in the dura of mice receiving CSF from injured mice compared to CSF obtained from sham donors (FIGS. 6I, 6J, and 6K). As CSF can be drained via meningeal lymphatics and into peripheral blood 24, we cannot entirely exclude the possibility that some of the observed response was systemically driven. However, neither a sham surgery nor a laminectomy resulted in the redistribution of CSF to a distant peripheral bone marrow niche in the tibia (FIGS. 10A, 10B, 10C, and 10D), suggesting that the phenotype observed was through direct CSF-to-skull-bone-marrow signaling.

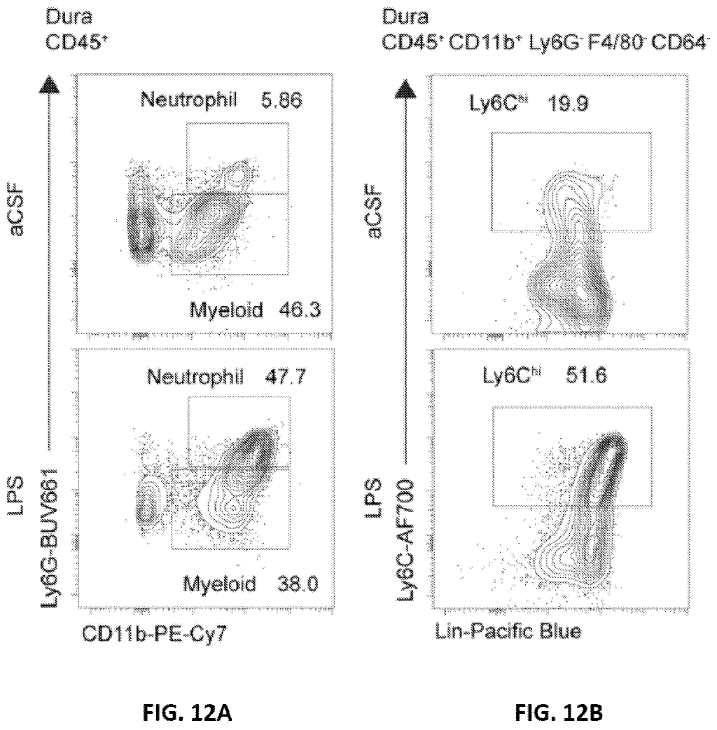

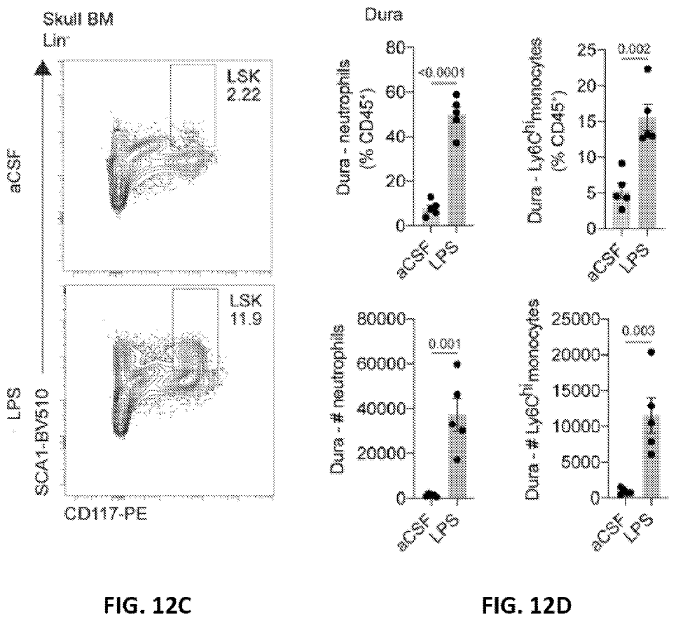

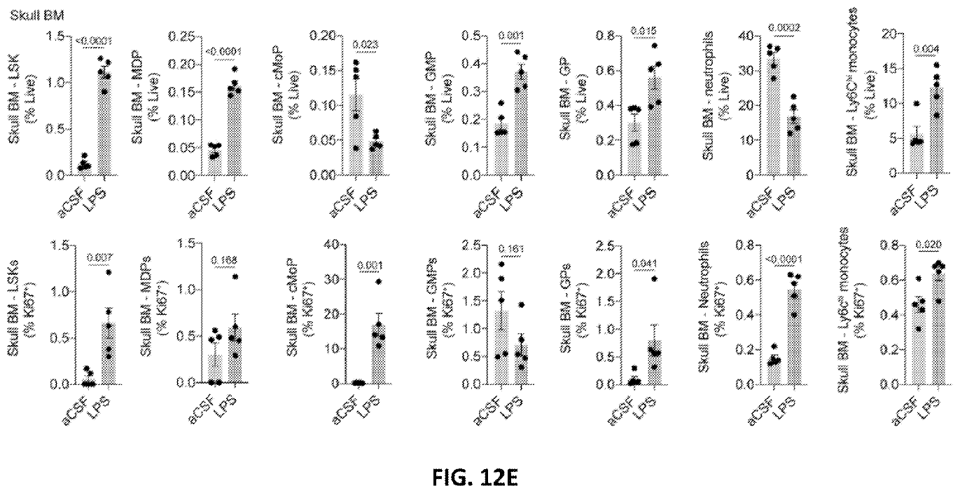

Beyond CNS injury, we hypothesized that direct CSF access to skull bone marrow could play an important role in CNS pathogen sensing. Indeed, injection of lipopolysaccharide (LPS), an outer cell membrane component of Gram-negative bacteria, into the CSF of mice resulted in the expansion of skull bone marrow HSCs and myeloid progenitors and a concomitant increase in dural monocytes and neutrophils (FIG. 12A, 12B, 12C, 12D, 12E).

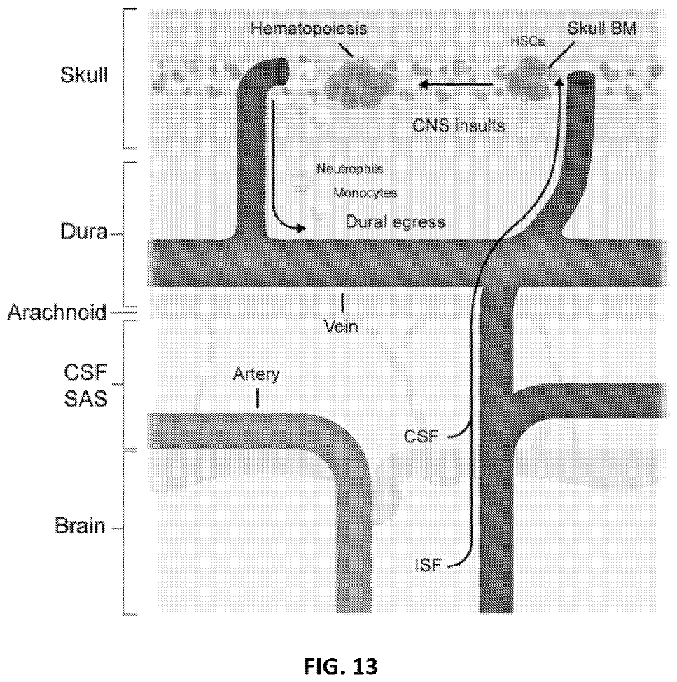

Understanding of the mechanisms regulating meningeal immune supply is evolving. In this study, we describe a previously unrecognized form of neuroimmune communication between the CNS and its surrounding immune reservoirs. We show that CSF accesses skull bone marrow niches and mobilizes HSCs and myeloid cells after CNS injury or infection (FIG. 13). Additionally, we show that skull bone marrow populations have a unique transcriptional identity compared to non-CNS-associated bone marrow, suggesting that CSF-derived factors might instruct the phenotype of skull bone marrow populations. Indeed, access of brain-derived antigens along with CSF to skull bone marrow may underlie the central tolerance of B cells educated in this niche.

Understanding how changes in CSF composition affect local immune supply from bone marrow niches will shed light on pathogenic mechanisms contributing to neurodevelopmental disorders, neurodegeneration, autoimmunity, and CNS cancers.

### Mice

Mice were housed under pathogen-free and temperature- and humidity-controlled conditions with a 12-hour light cycle. Mice were housed no more than five animals to a cage, with rodent chow and water provided ad libitum. In all experiments, male mice were used. Adult mice (8-12 weeks old) used in this study were C57BL/6J mice purchased from Jackson Laboratory (WT, JAX000664). Mice at different developmental stages (postnatal day 7 (P7), P14, and P21) were obtained from colonies established in-house. Aged mice (20-24 months old) were obtained from the National Institutes of Aging. All experiments were performed under the approval of the Institutional Animal Care and Use Committee at Washington University in St. Louis (200-043).

### Tracer Injection and CSF Collection

Mice were anesthetized via intraperitoneal injection of ketamine (100 mg kg-1) and xylazine (10 mg kg-1) in saline and placed on a stereotactic frame. The fur over the incision site was clipped, and the skin was disinfected with three alternating washes of alcohol and Betadine. For intracerebral injections, a midline incision was made along the scalp, exposing the dorsal skull. A burr hole was carefully made using a dental drill. A 1:1 ratio of OVA-594 and anti-c-Kit-PE (1 μl) was injected using a glass capillary attached to a microinjector (World Precision Instruments) over 2 minutes at the following coordinates: +1.5 A/P, −1.5 M/L, −2.5 D/V. The glass capillary was left in place for another 2 minutes to prevent backflow. For ICM injections, the posterior scalp and neck were shaved and prepared with iodine antiseptic. The head was placed in a stereotactic frame with the neck flexed. A midline incision was made and the posterior nuchal musculature divided, exposing the inferior, dorsal aspect of the occipital bone and the posterior dura overlying the cisterna magna. A glass capillary attached to a microinjector (World Precision Instruments) was used. Volumes of 1, 2, and 3 μl were infused in P7, P14, and P21 pups, respectively, whereas 5 μl was infused in adult and aged mice. Injection rates were adjusted to achieve a 5-minute injection, followed by a 5-minute wait period to prevent backflow. For CSF collection, a glass capillary was inserted through the dorsal dura mater into the superficial cisterna magna, and approximately 15 μl of CSF was drawn by capillary action. For CSF transfer experiments, 10 μl of CSF was transferred. For AMD3100 experiments, mice received a 10 μg injection in 5 μl of artificial cerebrospinal fluid (aCSF). For LPS injections, mice received 1.25 μg of LPS from Escherichia coli 0111:B4 (Sigma-Aldrich) dissolved in aCSF via an ICM injection. Mice were allowed to recover on a heating pad. For animals undergoing survival surgery, the skin was sutured, and ketoprofen (2-5 mg kg-1) was subcutaneously injected for postoperative analgesia.

### Tissue Processing and Immunohistochemistry

Tissues were drop-fixed in paraformaldehyde (4% w/v in PBS). Skulls, duras, and skull-dura whole mounts were fixed for 1 hour at 4° C. Bones were decalcified in OSTEOSOFT (Merck) for 3 days at 37° C. with agitation, and brains were cryopreserved in sucrose (30% w/v in PBS) until they sank. Tissues destined for sectioning were snap-frozen in an optimal cutting temperature medium (Thermo Fisher Scientific), and 20 μm sagittal sections were cut on a cryostat (Leica) and mounted onto Superfrost Plus slides (Thermo Fisher Scientific). Sections were blocked and permeabilized for 30 minutes in immunobuffer (PBS with 0.2% Triton X-100 (PBS-T) and 1% goat serum), and primary antibodies (Supplementary Table 2) diluted in immunobuffer were added overnight at 4° C. Samples were washed three times in PBS-T, and secondary antibodies (Supplementary Table 2) were added for 2 hours at room temperature. Sections were washed in PBS-T, counterstained with 4′,6-diamidino-2-phenylindole (DAPI, 0.5 μg ml-1, Thermo Fisher Scientific) for 10 minutes, and washed a final time in PBS-T. Sections were coverslipped with ProLong Gold Antifade Mountant (Thermo Fisher Scientific) and glass coverslips. Before imaging, skull-dura whole mounts were cleared in RapiClear 1.52 (SUNJin Lab) for 30 minutes.

### Confocal and Wide-Field Microscopy

Slides were stored at 4° C. until images were acquired using confocal microscopy (Leica, TCS SP8, or Leica, Stellaris) with ×5 (0.15 NA) or ×10 (0.4 NA), ×20 (0.75 NA) and ×40 (1.3 NA) objectives (Leica) and wide-field microscopy (Olympus SLIDEVIEW VS200) with a ×10 (0.4 NA) objective (Olympus) and a Hamamatsu ORCA-Flash 4.0 V3 digital camera. Quantitative analysis of Ly6b+ and CD3+ cell numbers was performed using the Fiji package for ImageJ (version 1.53c). Four to five representative images were acquired per site, per sample, and the results were averaged to generate the value used for a single mouse.

### Two-Photon Microscopy of Skull Bone Marrow

Mice were anesthetized via intraperitoneal administration of ketamine/xylazine and placed on a stereotactic frame. After ICM OVA-594 tracer (1 mg ml-1) injection, calvarial bone overlying marrow was exposed via midline skin incision. To visualize the vasculature, 100 μl of 70 kDa FITC-dextran (5 mg ml-1, Sigma-Aldrich) in saline was infused retro-orbitally immediately before imaging. Images of tracer in bone marrow niches of the intact skull were acquired in Nikon Elements 5.20 software using a Nikon A1RHD/MP system equipped with a ×16 water immersion objective (0.8 NA), resonant scanner, and non-descanned detectors. Excitation was achieved using a Coherent Chameleon Ultra II tuned to 820 nm. Fluorescence emission was detected using the following filters: 492 SP for second-harmonic generation, 525/50 for intravascular FITC-Dextran, and 575/25 for ICM OVA-594 (Thermo Fisher Scientific).

### Single-Cell Isolations

Mice were humanely euthanized with a lethal dose of Euthasol (10% v/v, intraperitoneal), followed by transcardiac perfusion of PBS supplemented with heparin (5 U ml-1). Skulls were cut from the foramen magnum at the back along the parietal ridge to the olfactory bulbs at the front and divided into dorsal and basal portions. A single tibia was taken from each mouse. All bones were cleaned by the removal of attached soft tissues, and the dura was peeled from the skulls. Bone marrow suspensions were obtained as described previously 8, with the skull undergoing mechanical dissociation by chopping with scissors and crushing with a pestle in PBS, and tibial bone marrow was obtained by flushing. All bone marrow cells were passed through a 70 μm cell strainer before centrifugation. Strained marrow samples were pelleted (450 g for 5 minutes) and resuspended in ACK lysis buffer (Quality Biological) for 5 minutes. Samples were pelleted (450 g for 5 minutes) and resuspended in fluorescence-activated cell sorting (FACS) buffer (2% BSA and 1 mM ethylenediamine tetraacetic acid (EDTA)). Dural meninges were peeled from the inner aspect of the skullcap and enzymatically dissociated in a pre-warmed buffer containing RPMI (Gibco) with collagenase VIII (1 mg ml-1, Sigma-Aldrich), DNase I (0.5 mg ml-1, Sigma-Aldrich) and FBS (2% v/v, Gibco). Meningeal samples were incubated at 37° C. for 20 minutes with five triturations with a P1000 pipette at 10 minutes. Samples were then resuspended and passed through a 70 μm cell strainer. The digestion process was neutralized by the addition of an equal volume of DMEM with 10% FBS (v/v). Dural suspensions were pelleted (450 g, 5 minutes) and resuspended in FACS buffer before staining.

### Flow Cytometry

Cell suspensions were prepared as described above and transferred into a V-bottom plate. Viability staining was performed using Zombie NIR (1:500 in PBS, 10 minutes, room temperature; BioLegend). Suspensions were then pelleted (450 g for 5 minutes) and resuspended in anti-CD 16/32 antibody (1:100, BioLegend) diluted in FACS buffer to block Fc receptor binding. Antibodies against cell surface epitopes were then added for 10 minutes at room temperature. For a full list of antibodies, see Supplementary Table 2. Flow cytometry was performed using an Aurora spectral flow cytometer (Cytek Biosciences), and data were analyzed with FlowJo (version 10, BD Biosciences).

### EdU and Ki-67 Staining for Proliferation Analysis

EdU staining was performed on selected flow cytometry samples with the Click-iT Plus EdU Alexa Fluor 647 Flow Cytometry Assay Kit (Thermo Fisher Scientific). Mice received intraperitoneal injection of 10 mg kg-1 of EdU 4 or 7 hours before sacrifice. After the single-cell isolations described above, cell surface antibody staining, fixation, and permeabilization, EdU staining was performed according to the manufacturer's instructions. Ki-67 staining was performed after EdU staining.

### CSF Collection and Proteomics

Mouse CSF (4 μl) was added to 16 μl of digestion buffer (100 mM Tris-HCl, pH 8, containing 8 M urea). The samples were reduced with 5 mM DTT and incubation at 37° C. for 1 hour. The reduced protein was alkylated with 10 mM iodoacetamide for 30 minutes at room temperature in the dark. The urea concentration was diluted to 2 M urea by the addition of 50 mM Tris, pH 8. The proteins were digested with LysC (1 μAU) for 2 hours at room temperature, followed by digestion with trypsin (1 μg) overnight. The filter units were then centrifuged at 14,000 g for 15 minutes to collect the peptides in the flow-through. The filters were washed with 50 μl of 100 mM ammonium bicarbonate buffer, and the wash was collected with the peptides. The peptides were acidified with trifluoroacetic acid (TFA) (1% final concentration) and desalted using two micro-tips (porous graphite carbon, BIOMETNT3CAR) (GlyGen) on a Beckman robot (Biomek NX)25. The peptides were eluted with 60% MeCN in 0.1% TFA and dried in a Speed-Vac (Thermo Fisher Scientific, Savant DNA 120 concentrator), after adding TFA to 5%. The peptides were dissolved in 20 μl of 1% MeCN in water. An aliquot (10%) was removed for quantification using the Pierce Quantitative Fluorometric Peptide Assay Kit (Thermo Fisher Scientific, 23290). The remaining peptides were transferred to autosampler vials (SUN-SRi, 200046), dried, and stored at −80° C. for liquid chromatography-mass spectrometry (LC-MS) analysis. LC-MS analysis and identification and quantification of proteins were performed as described previously.

Bone Marrow FACS and scRNA-Seq

Tibias and skulls were collected from five wild-type, 8-week-old male C57BL/6J mice, and the surrounding flesh was removed. For skulls, the dura was peeled and removed with fine forceps. Both the tibia and skull were then cut into small pieces using sterile scissors and mechanically dissociated in FACS buffer with a pestle, followed by a filtration step through a 70 μm cell strainer. Samples were centrifuged for 5 minutes at 420 g, and red blood cell lysis was performed with ACK lysis buffer. Samples were washed in FACS buffer, stained with DAPI (0.2 μg ml-1), and viable (DAPI−) single cells were sorted on a FACSAria II (BD Biosciences) into 1% BSA-coated 1.5 ml Eppendorf tubes with 500 μl of DMEM. Cells were then centrifuged at 420 g for 5 minutes and resuspended in 0.04% non-acetylated BSA in PBS, and viability was determined via trypan blue exclusion. Sample loading and library construction were performed using the 10× Genomics Chromium platform and Chromium Single Cell 3′ Library and Gel Bead Kit version 3 as previously described 4. Data preprocessing, dimensionality reduction and clustering, differential expression analysis, and pathway enrichment were performed as previously described 4.

### CSF Collection and Multiplex Analyte Analysis

Mice were anesthetized via intraperitoneal administration of ketamine/xylazine and placed on a stereotactic frame. CSF was collected from the cisterna magna under a dissection microscope using a glass capillary (Sutter Instrument, B100-50-10, pulled with a Sutter Instrument P-30 micropipette puller to a size of 0.5 mm in diameter). CSF (12.5 μl) was obtained from each mouse, and analyte quantification was performed using Luminex magnetic beads with the Bio-Plex Pro Mouse Cytokine Panel 23-plex instruction (Bio-Rad). Data were acquired with the Luminex FLEXMAP 3D and analyzed with xPONENT software version 4.2 (Luminex).

### Ligand-Receptor Interaction Network Analysis

The list of proteins identified in the CSF MS/MS (Supplementary Table 1) was converted to coding genes with the use of biomaRt using the UniProt ID and Ensembl gene name as conversion factors. This list was then filtered to include only genes contained in the list of ligands in the annotated reference provided by RNAMagnet21 with the function getLigandsReceptors with the cellularCompartment parameter set to ‘secreted’, ‘ECM’ or ‘both’ and the version set to 3.0.0. The receptors matching those ligands were mined from the reference, and their expression was plotted for cell types of interest as average normalized mRNA transcripts per population with the circlize 26 package in R.

### Spinal Cord Injury

Mice were anesthetized using ketamine (100 mg kg-1) and xylazine (10 mg kg-1). The skin over the upper thoracic area was shaved and cleaned with alternating Betadine solution and alcohol swabs. A midline incision was made and the paraspinal musculature divided, exposing the dorsal aspect of the spinal column. Hemostasis was achieved. A laminectomy was performed at T7 using a high-speed drill (Friedman-Pearson Rongeurs, 16221-14). At this juncture, control mice underwent closure of the muscles and skin in layers. Spinal cord injury cohort mice underwent spinal cord crush with fine forceps and then muscle and skin closure. Ketoprofen (2-5 mg kg-1) was subcutaneously injected for postoperative analgesia, and mice were euthanized after 3 hours.

### Statistics and Reproducibility

Statistical methods were not used to recalculate or pre-determine study sizes but were based on similar experiments previously published 4, 8, 17. Experiments were blinded, where possible, for at least one of the independent experiments. No data were excluded for analysis. For all experiments, animals from different cages were randomly assigned to different experimental groups. All experiments were replicated in at least two independent experiments of at least five mice per group, and all replication was successful. For all representative images shown, images are representative of at least three independent experiments. Statistical tests for each experiment are provided in the respective figure legends. Data distribution was assumed to be normal, but this was not formally tested. In all cases, measurements were taken from distinct samples. Statistical analysis was performed using Prism (version 8.0, GraphPad Software).

### Example 1: Role of Ly6C+ Monocytes in Treatment of a CNS Injury or Disorder

To characterize the role of Ly6C+ monocytes in the treatment of a CNS injury or disorder, the following experiments are conducted.

Additional details regarding these experiments are provided in Appendix A, incorporated by reference herein in its entirety.

What is claimed is:

1. A method of treating, preventing, or reversing a CNS injury or disorder in a subject in need by administration of a therapeutically effective amount of meningeal immune cells.

2. The method of claim 1, wherein the meningeal immune cells comprise Ly6C+ monocytes.

3. The method of claim 1, wherein the meningeal immune cells are modified to include a drug or gene therapeutic compound, wherein the modified meningeal immune cells are configured to deliver the drug or gene therapeutic compound to the CNS of the subject via the parenchyma or meningeal tissue of the subject.

4. The method of claim 1, wherein the meningeal immune cells are administered to the subject by infusing or engrafting.

5. The method of claim 1, wherein the CNS injury or disorder comprises at least one of CNS injury, autoimmunity, infection, aging, and chronic neurodegeneration.

6. A composition for treating, preventing, or reversing a CNS injury or disorder in a subject in need, the composition comprising a therapeutically effective amount of meningeal immune cells.

7. The composition of claim 6, wherein the meningeal immune cells comprise Ly6C+ monocytes.

8. The method of claim 6, wherein the meningeal immune cells are modified to include a drug or gene therapeutic compound, wherein the modified meningeal immune cells are configured to deliver the drug or gene therapeutic compound to the CNS of the subject via the parenchyma or meningeal tissue of the subject.
